SEQUENCE LISTING (31 sequences)

SEQ ID NO: 1 - DNA, 124758 bp, Varicella-zoster virus
ggcccagccctctcgcggccccctcgagagagaaaaaaaaaagcgaccccacctccccgcgcgtttgcggggcgaccatcgggggggatgggattttttgccgggaaaccccccccgccagcctttaacaaaacccgcgccttttgcgtccacccctcgtttactgctcggatggccaccgtgcactactcccgccgacctgggaccccgccggtcaccctcacgtcgtcccccggcatggatgacgttgcgacccccattccctacctacccacatacgccgaggccgtggcagacgcgccccccccttacagaagccgcgagagtctggtgttctccccgcctctttttcctcacgtggagaatggcaccacccaacagtcttacgattgcctagactgcgcttatgatggaatccacagacttcagctggcttttctaagaattcgcaaatgctgtgtaccggcttttttaattctttttggtattctcacccttactgctgtcgtggtcgccattgttgccgtttttcccgaggaacctcccaactcaactacacgaaactactgtccggaaggggaaggtatttattctcgcttgcagcttgtcgcgcgtgtatgcacaacaaaagctatatatgtcaccaaagccaacgtcgccatctggagtactacacccagtacattgcataacctgtccatctgcattttcagttgcgcggacgcctttctccgggatcgtggccttgggacatcaaccagcggaataagaaccgccggtggtcttgcccgaacgacgagtggcgacgcgttgttctgcataagctctgtatgctgatacataaacacagagtctgtatcgctatcagattcccgaacaccttccggtaccccatactccgataccctggacattgcggatcccaaaaatataatattaacaggatttgcttatactttgctacagcttatataaatttatgtgcgatacatcttaagtgcatccgtacgttatttatacattgcctgtcacgtgaaaagactgtgttacccaataaaggttctacaaaaaatgctttattgggtgtttgtttaatagctattatcgtaacccacccccgtaaaatcataaaatgcatgtaatttctgagacacttgcatatgggcatgttcccgcatttattatgggctccactctggtgcgtcccagtttaaacgccaccgccgaggaaaatcccgcgtcagaaacgcgatgtttattacgagtgcttgcggggagaactgtagacctgccaggcggaggaacgttacacattacctgtaccaaaacctatgtaattattggcaaatatagcaaacccggcgaacgtcttagccttgcccgtctaatagggcgtgcaatgacgcctggaggtgcaaggacatttattattttggcgatgaaggaaaagcgatccacaacgcttgggtatgaatgtggtacgggcttgcatttactggctccatctatgggtacatttctccgcacacacggtttaagtaacagagatctctgtttatggcggggtaatatttatgatatgcatatgcaacgtcttatgttttgggagaatatcgcgcaaaataccactgaaacaccttgtataacgtcgacgttaacatgcaacttgacagaagactctggtgaagccgcacttaccacgtcagaccgacccactctcccaaccctaacagcccaaggaagaccaacagtttccaacattcgtggaatattgaaaggatccccccgtcaacagccggtctgtcaccgggttagatttgccgaacctacggagggcgtattgatgtaatcactaaataaaatacaccttttttcgattgtacgtatttttatttaaatgtgtagttcatagtccgccgacagccgctcgggcttttcccccacatacaacatgatcgtatgcctcggatgcaccggtccaacactccgccgagaagggggatttacaatgacagtgatacccaatagccgccagatgtacacccagctgtccggactccagcatcatctgctgagttgcggcgctgaagggtgcatcgcatagggtgttataattagccatttccggtaacagtcgttgggaatttaggaggctgcaaaacggctgtaggtcaacatacattggggattcagatggtttatctcgacgtccaagtccaatcaaaaaagcgtgtaaatcatcagcccggccgcatgttgctcgaagagcacataacctcttaacaccgtacagaggggatggcgtcggtgcatgtgagttggcagggcatgtccacgttgtttccaacgccagtggcggtataacttgtgtaaacgacgccaacgggtcaggtttaagattcactcggatgggttgactgctttcggaagctcccgttgtatccattaattaaacgttcggtacacgtctggtgtgtgttttacccgaatcagagacggaattgcaaagatatcggtttgaaagcaatgtaatcccgcccatatatccccaacgtcgccttaaaaactcccacaatattacatttttattagtcttttattaatatagaatcacataaacaattgataaaatcaaggggtggtgtataatgattaaaaatataaattgatatgttttacaagcatgaaataggtatttactattctaacaggtaaatatgcttaatgattaaaaatacaaattagtatgttttgacaagcatgaaaaaggtattttttattttagcagttaaaggtactacacttaaaatatttaccgtatggacgggcgtcagaaagatgcccggcccaagttgagagggtacattcaacacgaccacactcgcgttggtgggtgattagggcctctaaaacaccggccagacatgacccgggtgtatattcttgtaacacttgaacgttacaactgatatcatcatattccacaaatttagagccacggacaactatattagcaatgcgggcaatcataacaaacatataagtagtaatacacgtgatatcactaaaacgttgctggcgcaacagttcggggagagtacgagaccccaaatcgttgtccctgtttagaagaagacatcttacaaaaggccccagctttaactttaaattctccaaaagtgacttcgaggttgcaacaatgggattatttgtgtagatgggcaagttttttgccgctaacattttaatccacgttaacagttcatccgcagactccaacgcttcaatcaaagattctccacgtatgactctctcacgcaacgcgcgggcaatacgtgagtccattttatatgactcaaaggtacgataaagttcatgtccgtacaacatcaactccggccaagatgtgttttgttttatccccggaaaacatccaccggaagcccatgaatcaccctcttgtattgtggcatatcggactaccagtttttcaattgtttcatctaaatggcgtaccgagtcaatggtcacgctggctcccgcggtggagacgacttcaatagcacggcccgtaattcgatcgaccgggatatcatactcttttcgaatacgctctcggcgggcgtctctcttggaaaatcgcaacctgtacgattcgtcatgtgtctgatcatttctttctcccgtggtcattgcaggaggcgttgtaggacgccgtcttcgatttgacagggatcgatcacggtgttttcttgaactttgggtgttataagatctggatgatcgtcgatgtccccgttcgatgcgtgcatatccagtctccacgtctcttcctccatgatggtttgaatcgggtaatacaacaaccaaagttttcgggcgattgtggtggtagctttcacgccttccgtgccttcgtttggaataccgtggattatatgctgtatctgcagtacgctccacatacacagttctagacgttgtggagtcctcgcctggagtggagccaatagcttcatcatttgcccaatcggtgacttccaatgcaaagtcatccgaaggttcgtctggtagcaaattcataaagtcttcacaaatagtagacacgtctgggtcggttggaattgaagcagaggccatggctgcaaaatatctgacaattgcgtgtttgcagttgcctgtatcttccgccaatgttgtagaatttataggctcacccaaccccgcaatgggcgtgtttagtcacatgattaatgtttctgggagttttcactttccccaaacaagcttacctgcaccctttgttcgtaatgcataaaaataaccactgctatagcaaatatgacgatataaaaacattttatagcaaggccggacattactgtagcgcaacatgttgtgcatataccacgtattccccccgtattgatatgatttaaatgattatccttggttggttttggtctaacataagatataagctctactatagcgagcgtgcatacaacaacccaggccagaatccgaatgtatgtggggtataataacgcgcatggtgtatatgcaacgccaagcgttaaaagcacaatacatccagatgatatatgagcgataacctccaaaagcatcaataacgtaacacctttatgcatatataaaaaacttatagggtcagcattaaatactttactcataccatcccgtcgcatggaaacatcacataacaaccttgccaactttgtatatgggtaaccaagaagaatgttcgaaataacccgtgttacgtaattcagtgaatatgatgtgggggatattaactcacaggatgatcggaatggcccaaacatacgacgtattcgtcgaaattgtaaatacataccatatacaaaccatgcaaaaaaaatcatttttagctgcacgcaccaaaaataagcgtgacaattacgtgttcccagaacaattcgaattttgtcatgcaaaggtgtagaaatagcggtttttaccatagtatctcctgataatagattttcccggcagctgtaatcgtatccagataggccatccaaaaacgttgagtggtttacaaacgttacatatataagagagttgttataagacccccatacaaccggtccaccattaatcaccgtggttgcatacacacactcatgttcaaactttacacgagcggtataccatagggtaaaaacagcatgtccgctaagtagacacataattataaaatgttctgtcttgattcctaaagcctgcatgacccgtggaagatggcaattcaagcacgatgtagtatcacacggttggtgttaactcgaagttaaatttggataattaggtacttctagagtaaagattgtatgcatgcgattgctatcgcactttgtagcaaaacattgttgtgcaagcgaaatacacaaacggttgtgatgatccactcgcagagacacaaatgtccggggagccgttcttcctccgcgatggggatatcgaagacaagtgaacccttttgttccgcatatgagctgaaataacacccagtcccttttgatggcgatacactttgatgatgttaaggtatattcgcgatcacgcccggggaaatgaacagcaatatgctccacaatagattctaatattgtgctgtcgacaaaggcctccagtgtaaatgcgtccagacaagttaccccgcgctcttttagagcctttgttaaagatatttgcggggggctaaatatttgtttattacgcgcaaccttacgttcaaaaaactctgcgtattcccccccaaggttatgtaaaataaattgcactggaacattcgactgcggtcttgaatgaaaatgaaagtttgccgggtttctatgtgatgtcacaaacgctaatatatcaatacactgctcaggtacaacataaaatgggagtagttgtccaaccgccgtccctgtggttgttactttggagaaaaaaggcagtcttaaactatgtccgtggctataaacaccagtatctataaacgaaaagtcccgtaaatacggaccaatatattcaacaaattcccgttccagcaacaccgcttgctgtaatatttgtgcaaacccctttaaagtggaagaccccactaacgcatagggatttgggattggtacgcataccctgaaacctattttctctttacagttacagggtagagtttcatgcaagttttcattgtttgatacatcggcgtgtgtatggacttcagacgttgtctgtgtatcaaaaaaccatacatcctctgtataattctcttctacacacgtgtataattcgccattttctatgtaaaaatcgatgtcagaatggctggttatatccaataaattatcatcatccaacacctcaacggtaggttcaggacatgcagttttataaaaataacatgggtctttgttagggtttaccacggcctttggaaaaagtaattgcatggccgttaaaataccatgacgaaatgctcgcatgccggcatgtaaaatacccaatgggatgggttttcttatatgaaagtctacatcaagtatgaggtttgtgattataagatttgtattaaatagctcattcctgtttatataaagctgatctttgggtatgtttgatgaaattttagaaacgtttttaacagacgtagataatagtaaagtcaactgcatatctcgtagtgaagcggcaacaaaattacatggattaatttgtttaaggtcctccgcaattaatcgagcctcgtgcggtaaagtgtaacggtttgttattgatgaccacgtatcattagcaataacagcaaatgcttgggcgccttgaggcaaggctacccgatatacaggcattggtccagttacctcagaatggccgatgagggcttctaatggagttttataactcaggatggatacatcatgtgtggctatcccagtggcagcagagaaaaacagtaatagttttgtaatccccgggctcgtatcaaaaccagtacgaccactttggttaggtgtatcgtttgcaaagttggctgctcgtaacgcctccgcggaaacaaccgaatcctcaaaattagacaattcgtcaaaaccgggtggatttgagggaatagtggaggaccatccatatggactaaattgtttttcaatgttttccacacgacgagttagcgttgtagctaggtcacatacgcctataaacttgctaggttttgcggcatacgtaagacttaaagtatatgttttagtaattgtatatttatgtccaatctcaggtccaagttcagtgacatcacaaattacgttcttttttatatagtcacgcatgttgagacgagaacgtacatgattaaaaaaattagcagtagctctttttcccaggttggatgattttaagaggaccggtttattcacaaaatctgagtatgtaaccgcttgtaggtggtctgcgatctgtttccgattgaaacattcaaaatgtgccagataaatataatcaacaaattcacggtctggaactttaaggccttttctatcgttggtaatatactccgatactgcgtgtatttccgttgtgtctgtatgtattcgctgtaaaatgtacgatagagcatttttggctgtcaaacctcgtgtatatgttgaggaacaacaaaacatggaaagtttatcaaaagacaacaagtccgaaatattgtacccactacaattaggtaatgctgggacttggtaagttaaaaacaaatctttaattgcctgtaagtcatataagggggtttccaacgtattgtaacttgtgtccgtttgtaacaagtaatagcgtgtagccaacactagcgttttttcagagggtccaaatcgaacaatataccaaaacggcgagcatccatacccccagtagagtcgtcgatatgcagccaatacttgacgttcgtaatgggcatataatgatgttagctcctgacgaccaacggattttttaactaacttgcagagtgttgcctctgtgatgcataggccgttgtccgataatccctttcggtttaaatggtgtgttgttaccatcagagtttgtataacttccgagtgaatgtcaaacgtctccgatatacatagggtatcagatattatatgcggatttaggggtgctccataccataacgccttatataaagctttaaaatcagtttgggttttaaaacaacaaaaaaatataggccagacccgggatcgtacatctccagttgaaaatccaccaattaaataaaaaataacgttgacgtccctactacaaaataaatgcattatttggttttcttcatcgttttcagttacttcacgtgggcgtttagttgggattacttgcgtgatctcttccctcccatttttgacaaagacgtcatctaagtcgggagtccaagtataactcaccacatacagaggttctgtgcttatctgcccggtaagcaacaacagcgagtgggagattgcacatccctttgtggcaaataataaccgaatcgtcggtttggaggatttatccatagttcaatacgttggaaagccagtcaatcatgcagacggtgtgtgccagcttatgtggatatgctcgaataccaactgaagagccatcttatgaagaggtgcgtgtaaacacgcacccccaaggagccgccctgctccgcctccaagaggctttaaccgctgtgaatggattattgcctgcacctctaacgttagaagacgtagtcgcttctgcagataatacccgtcgtttggtccgcgcccaggctttggcgcgaacttacgctgcatgttctcgtaacattgaatgtttaaaacagcaccattttactgaagataaccccggtcttaacgccgtggtccgttcacacatggaaaactcaaaacggcttgctgatatgtgtttagctgcaattacccatttgtatttatcggttggcgcggtggatgttactacggatgatattgtcgatcaaaccctgagaatgaccgctgaaagtgaagtggtcatgtctgatgttgttcttttggagaaaactcttggggtcgttgctaaacctcaggcatcgtttgatgtttcccacaaccatgaattatctatagctaaaggggaaaatgtgggtttaaaaacatcacctattaaatcggaggcgacacaattatctgaaattaaacccccacttatagaagtatcggataataacacatctaacctaacaaaaaaaacgtatccgacagaaactcttcagcccgtgttgaccccaaaacagacgcaagatgtacaacgcacaacccccgcgatcaagaaatcccatgttatgcttgtataaatattgaaataaaaactaaaaacgtttctggtgtatgtttttattttgtatataaaattaaaacattgctggccggcgtggttattacatttaatgttttagtagaaaatcgacatcgtttgtttctttatcagttgaaccaaatccacgcgttccccgttcgctgggtgtggctattagatctaacgttttagtaaaataccattgtacacccggtatgccacatttaccgcggatagcataaggaaatgcaatattacttaaaacgttgtgttttaagtgtatttgggtgttgtgatctattaacaggacctgtgcaagacgatctcccgtttttatacgtatgtcatcacccgtgagattatatacgtagaatttacagtgttctcctgcaggccatgccgttggacacacgataatgcctgatcggcttttcgatgatcttccaaaaatataagcgtttatactcggatgttgtaagtcccagtctcttataatcggtaagacaatttttataaattcattcctttttaaatataggttatatggtacacaaatatcatatcccgcgtcttcttggcgttttggattgatgatatgtttgtaggttaagggaacatcgatatggtattctgcagaatccctatgtaaaggttgcccctgctgtaccgtggaaatatcagcaaattcagatataacgggtttttcataatttgacggcgagtttgataagggttgaacttgtatcgatttaaaaattggatccagatgtttaagaacgttttttgggagaaggcgactttgtcttaattttaccgggaacaagtagattgttaaatgtccgggtaaaataacggttactcctggccggtaatacaaaagggctgaaattactcctctgtaacccgcatcaataactccgttggcgacaaaaaaattgtcttcatcagcaagggcagtatctttgcattgaattaacaacagtgcgtattcattgggaggcgccgacttaaccaacagctccaactgctgcatataaaaaccgccccgtgttacagatttttcagatggcagttcgagtttcttgtggttccggagtaacaacggttgatgtcgacttactttatcgtctaacacgcattgcagcgtatctgcacattcaggttgaacttctattaaaattgtatcttttaaacaccgattcggaatagtttggctacaaaacatatcacctgtatttactgccgtttccaagatgggatcaattaccgcttcgttcatattaataacgatgcaaattttatttttttgtgaagacagcagtggggagccaaactttgcagaacggaatttttggcatgccagctgttcggctcgtggagtttatatcgacggatcaatgatcaccacccttttcttctacgcatcccttttgggggtgtgtgtagcccttatttcgttagcttatcatgcgtgtttccggttatttactcgttctgtattacgcagcacgtggtaaacccgtttgcctataaaaggggcaggcgtgtataagagggcccctgtttaatacgcggtctgccgtgtttggatatttcacgaccctatcgtttatttacgtaatggcatcttccgacggtgacagactttgtcgctctaatgcagtgcgtcgtaaaacaacgcctagttattccggacaatatcgaaccgcgcggcgaagtgtggtcgtaggaccccccgatgattcagacgactcgttgggttacattaccacagttggggccgattctccttctccagtgtacgcggatctttattttgaacataaaaatacgacccctcgcgtacatcaaccaaacgactccagcggatcggaagatgactttgaagacatcgatgaagtagtggccgcctttcgggaggcccgtttgagacatgaactggttgaagatgctgtatatgaaaacccgctaagtgtagaaaaaccatctagatcttttactaaaaatgcggcggttaaacctaaattagaggattcaccgaagcgagctcccccgggagcaggcgcaattgccagcgggagaccaatttccttcagcactgcaccaaaaaccgcaacaagctcgtggtgcggtcctacgccatcatataacaaacgcgtcttttgtgaagcggtccggcgcgtagccgccatgcaggcacaaaaggctgccgaagcggcttggaatagtaatcccccaaggaataacgccgaattagaccgtttgttaaccggagccgttattcgtattacggtgcatgagggtttaaatttaatacaagccgctaatgaagcagacctaggtgaaggagcatcggtatccaaacgtggacataatcgaaaaactggagatttacaggggggcatgggtaatgaacctatgtacgcacaagttcgtaagccaaaaagtcgaacggatacacaaacgactgggcgtataactaatcgaagtagggcccgttcggcatcaagaactgatgcgcgaaaatagggatataattacgcagtaacggtttacccggtattatgtataataaataaacgtataaaagacagtcgtggtttgtgtttattataaatgtgtattatatgtcacatattataaactgtttaaatagtaccacgtggtattatgaacagtttataatcagttgctaccaaacaaaccccattagacggcgggttttgataaagggaatcgcttatttaaactaaagattttactctataagtatggagtgtaatttaggaaccgaacatcatagtacagatacgtggaatcgtagtaaaacggaacaagcggttgtggacgcatttgatgaatcgttgtttggtgatgtagcatcggatattggatccgaaacgtcgttatattcacatgcagttaaaactgctccgtctccgccttgggtagctagccctaaaattttatatcaacagttaatacgggatcttgatttttcagaagggccgcgtttactatcatgtcttgaaacctggaacgaggatttattctcatgttttcctattaatgaggacctatattccgatatgatggttttatccccggatccagatgacgttatctcaaccgtttcaaccaaagaccatgttgaaatgtttaatttaacaacccggggttccgttcgattgcctagtccaccaaagcaaccgacggggcttccagcttacgttcaggaggtccaggattcgtttaccgtagaactacgcgcccgggaagaagcatacacaaaactactagttacttattgtaaatcgattatacgttatctccaaggaacggcgaaaaggacgacaataggtcttaatatacaaaaccctgaccagaaagcttacacgcaactcaggcaaagtattctacttagatattatcgtgaggtggcaagtttggcgcgtcttctgtacctacatttatatttaaccgtaacgcgtgaattttcctggcgtttgtacgccagtcaatctgcacacccggacgtgtttgcggctttaaaattcacctggaccgaacgtcgacagttcacgtgtgcgtttcatcctgtattatgcaaccacggcattgtgttattagaagggaaaccactaacagcgtctgccttgagggaaataaattaccgccgccgagaactgggactgcctctagttagatgtggtcttgttgaagaaaacaaatctccgttggttcaacaaccctcattttcggttcatttaccacggtcggtggggtttcttacccaccacattaagcgtaagttagacgcatatgcggtcaaacatcctcaagaaccgagacatgtacgagcggatcatccttacgcaaaagttgttgaaaatagaaactacggtagtagcatcgaagctatgattttagcacctccgtccccatccgagatcctgccgggggacccaccacgcccacccacgtgtgggtttttaacgcgttaaacgtcattggggtaaagggtgtaaataaattacgaaaacgtgcatgcgttttttatttttacaatgcgccgtatatggtatgtctgtcatgtgctctaaagtcccatatataaaagaagccccaacgagtgtatgcgtattgcgtaccgcgaccctgggatgttttacaggcgcgtttgtttgtctcggttataagtatgcagtcgggtcattataaccggaggcaatcccgccgacagcggatatcgtctaataccacagactccccccgtcacacacacggaacacgttatcggtcaaccaattggtatacacacccaccccagatattgtccaattcagaaacattagttgcggttcaagaactactgaactccgagatggatcaggacagcagttctgacgcatcggatgattttccgggatacgccttacatcattctacatataatggatccgaacaaaatacatcaacttccagacatgaaaatcgcatatttaaattaacggagagggaagctaatgaggaaatcaacatcaatacggacgcgatcgacgacgagggagaggcggaggagggagaggcggaggaggacgcgatcgacgacgagggagaggcggaggagggagaggcggaggaggacgcgatcgacgacgagggagaggcggaggagggagaggcggaggaggacgcgatcgacgacgagggagaggcggaggagggagaggcggaggaggacgcgatcgacgacgagggagaggcggaggagggagaggcggaggaggacgcgatcgacgacgagggagaggcggaggaggattatttttctgtaagtcaagtttgcagtcgagacgcggatgaggtttattttacgttagacccggaaataagttacagtaccgatcttcgcattgcaaaggttatggagcctgcggtatcaaaggaacttaatgtatcaaaacgttgcgttgaacctgttaccctaacaggctctatgttagcgcataatgggtttgatgagtcctggtttgctatgcgcgaatgtacccgtcgcgaatatattacggtccaaggattatacgacccaattcatttacggtatcagtttgatacttcccggatgacacccccacagattttgagaactataccagcccttcctaacatgacacttggtgaacttttattgatttttcctattgaatttatggcccagccaatttctatagaacgtattttagttgaagatgtatttttagataggcgggcttccagtaaaacacataaatacggcccgcgttggaattccgtctacgcacttccatataatgcgggtaaaatgtatgtacaacacattcctgggttttatgacgtgtccttacgtgctgtgggccaaggaacggccatttggcatcacatgatattatccacagcagcatgcgctatttctaatcgcatttcacatggagatggattaggatttttgttagacgcggcaattcgtattagcgcaaactgtatttttttgggacgtaacgataattttggcgtgggggatccatgttggttagaagaccatcttgccggattaccacgagaagccgtacccgacgtactccaagtgacacagttggttttgccaaatcggggtccaacggttgccattatgcgtggtttttttggggcgttggcatattggcccgaactaagaattgctataagtgaaccatctacatctttggtgcgatatgctaccggtcacatggaacttgccgaatggtttttattttcacgtacacatagtttaaagccacaatttaccccaacggaacgggaaatgttagcgtcattttttacgttgtatgttactcttggtggaggaatgttgaactggatctgtagagcaactgcaatgtatttagctgctccttaccattcccgttcggcttacatcgcggtctgtgaatctctgccctattactatatcccggttaatagtgacctgttatgtgatttagaggtattactgttaggcgaggtcgacctcccaactgtttgtgaatcctacgcaactattgcacacgaattaaccggatatgaggctgttcgcacagcagccacaaattttatgatagagtttgccgattgttataaggaaagtgagaccgatttaatggtaagcgcgtacctgggggccgttttattgttacaacgggtgttgggtcatgcaaatcttcttttgttgcttctctccggtgctgcgttgtacggaggatgttcaatttacatcccccgaggtattttagatgcatataatactttaatgttggcagcaagtcctctttacgctcaccaaactttaacatccttttggaaagaccgcgatgatgcaatgcaaactttggggattcgaccgacaacggacgttttacccaaagagcaagacaggatagttcaggcatcacctatagagatgaacttccgttttgtgggattggagaccatctatccccgagaacagcccattccctccgtggacctagccgaaaatcttatgcaatacaggaatgaaattctgggtttggattggaaaagcgtagccatgcatttactacgaaaatattaagggttgtgatttttttcattaggatgaaaagaacgtttcctagccacacccacaaaggagtttgtaaaataaaatctctgtttagaccttaaaatttgttgtgtgtgttgtgtggggggtccgtgaggatcgacctttacaagatataatttgtccatatcgcaatgttttctcggtttgcgcgttccttttccagcgatgatagaacgcgtaaatcttatgatggtagttaccaaagttttaatgccggcgaacgtgatttgcccacacctacccgggactggtgttctatttcccaacgcataaccagcgagcgcgtgagggatggatgtcttattccaacgcccggcgaggctttggagacggcggtaaaggctttatctgaaaagaccgacagcctaacatcgccggttttacaaagtaccgaaagacacagtgttctgcttggattacaccataataatgttcctgaatcgttggtggtctcgtgtatgtctaacgatgttcatgacgggtttatgcagcgttatatggaaacaattcaaagatgtttggatgacctgaaactttctggggatggactttggtgggtttatgaaaatacatattggcagtatctcaaatacaccacaggagccgaggtaccggtgacttcagagaaggtaaataaaaagtctaaatccacggttttgttgttttcatccgtagttgccaataaaccaatatccagacatccttttaaatctaaagttataaattcggattaccggggaatatgtcaggagctacgtgaggcgttaggagctgtgcaaaagtatatgtattttatgcgtccagatgatcctacaaaccccagcccggatacaagaatacgtgtacaagaaattgcggcttacacggctactggctacgggtggatgttatggttcttggacgttgtggacgccagggtatgtcgccatctcaaacttcaatttcgacggattcgagggccgcgcgcgtctgttattccagatgatttgcttagacgacatttaaaaacgggtcctgcggtctcagcgggcacaggagttgcgtttattttagcagcaacaactgccagcgctcttactgcgcttttgcgtattagtgtattatggcgaaaggaagagtggcgggatggtttaaatggaaccgcagctgcaattgttgcggcggttgaacttattacgcttttgcaccaccattttcaatacttaattaatatgatgcttattggatatgcatgttggggggatgggggattaaacgatccttatatattaaaggcgctacgtgcccagggacggtttttatattttgcgggtcagttggttagaacaatgtcaacacacagttgggttgtgttagagaccagcacccatatgtggttttcccgggccgtggcgcagagtattttagcacatgggggtaaacccacaaagtattatgctcaggttcttgccgccagtaaacggtatactccgttacatttaagacgtatatccgaaccatcgagtgtgtctgatcagccgtatattcgttttaatcgactgggatctccaatagggacaggtatagggaatttggaatgtgtctgtttaacgggaaattatttatctgacgacgtaaatgcaagttcgcatgtaattaatacagaagcaccgttaaacagtatagcacccgatacaaatagacagcggacttctcgcgttttagttcgtccagacacgggtttggatgtaactgtccgaaaaaaccactgtttggacataggccatacggacggtagtccagttgacccaacgtatcctgatcattacacccggataaaggcggaatatgaaggtccggttcgggatgaatcaaacacaatgtttgaccaaagatcggatttacgtcacatagaaacccaagcatctttaaatgatcacgtatatgaaaatataccacccaaggaagtgggttttaactcatcttcagacctggatgtggatagccttaacgggtacacttccggagacatgcatacagacgatgacttatcaccagattttatacccaacgacgttcccgttagatgtaaaaccacggttacgtttaggaaaaatacgcctaagagtcatcattaagtacagcggttaatagatagttatggactaggcactttggcggtcatttccacaaccaggttaaaattgggggatttgggagaaaatagtctattgcgtattttctgttcaataattggactgcgttatttaaaggtctgattggttgattgggttataaaaggaattactcctttaaattttacttaatgtacccacaatatcaagtggtcgtttgtatttaacgattattaccggtaccatgggagacttgtcatgttggacaaaagtgccgggttttacgttaaccggcgaacttcagtacttaaaacaagtggatgatattttaaggtatggagttcggaaacgcgatcgaacaggaatcggaacgttatctttatttggaatgcaagctcgatacaatttgcgaaatgaatttcctcttttaactacaaagcgtgttttttggagggccgtcgtggaagagttgttatggtttatccgcgggtcaaccgattccaaagaactcgccgctaaagatatacacatatgggatatatacggatcgagcaaatttctaaataggaatggcttccataaaagacacacgggggaccttggccccatttacggcttccagtggagacattttggagcggaatataaagactgtcaatcaaactatttacagcaaggaatcgatcagctgcaaactgttatagatacaattaaaacaaacccagaaagccgacgaatgattatatcgtcttggaatccaaaggatatccccttaatggtactacctccatgtcacacgttatgtcagttttacgttgcaaacggtgaattatcctgccaagtataccagagatcgggggatatgggccttggggtaccgttcaacattgctggatatgcacttcttacctacatagtagcgcatgttacaggacttaaaaccggagatttaattcatacaatgggggatgcacatatttacttgaatcatatagatgctttaaaagtgcagctagctcgatccccaaaaccttttccttgccttaaaattattcgaaatgtaacagatataaacgactttaaatgggacgattttcagcttgatggatataatccacacccccccctaaaaatggaaatggctctttaatggatttttaaatgttgtcaagacagtagatgtgttgcgaatgtaataaaatgatatacacagacgcgtttggttggtttctgtttatgaacagcaacggatgcatagggttgcgataactgcgataagacccaatgtcccaaggatagatatcacaccaattataactgctacaacggaaaatgtagtggcgtaggtagatgcatcgtaggtataaacggccgaaaacggagggaattttttagggtaaccatctagatgacacgaataggtgataggtccgtcgagttccgatgttggacaagaactttgcatgtttacaaaccgtttgttttgatcacacaccccagtaatctcactgttttcgtggttgatgggagaatcgttaacccaccatacgaaatgtacaacgccacgtggcacacattttgccgtacatactatgtgtccatcaataatacctatagacacgttgggaaatggatagacgtcaggggtaacgacagcagaatatttcatattagagacgccatcccgaatccataaaacattacattggatggctgggggtgggtaatccatttgtttttgctgtggaattcgtaccgccgaaacataactaaataatccattggcatattcttgtattgcatcggttataaaattttttccgatgttaccaaaccttgaagtccaccgaacacgtaccgagtgcggtggataatactttgatacgttacagtaggctgcgtatgtctgtccggttaagactggatcgccgacaacggtaatatttggacgataatacgttgtaactgtaatactgtgttccgatatgacgttcttagtttttgtattaacgactcgccaaatatacgttccctccgtggtagcatccatagataaaattgttacagaaaaatcagacgttgttttaacatctggtattacataattttccttagcgtgtgtaaatatctcagggttgtttattaagtttaaatcggcactgttgctatataacataaccggtaaatctggcatgcgtattaacgcattgcccagttgacggtgcggatctataaggtgacgcgtaaaccaaacttcaatatgaagatcggggcgtataagcgacttccaccttgttatatttgaaccttccggatctaaagaatattgttcatatgttttttgttgctgcttaaaggccgcctgttgtccggtcgttagacgcatgtaacaaggcatgataaatgtgtgaaaatagggtatggattgtattccgccgtgaacgcattgtatattttcaaatagaaaaggtggttgtgaatgttgggcgttggctgcgggatcgggctttcgggtagcggccgaggtgggcgcgacggcgggatcgggatttcgggaagcggccgaggtgggcgcgacggcgggatcgggatttcgggaagcggccgaggtgggcgcgacggcgggatcgggatttcgggaagcggccgaggtgggcgcgacggcgggatcgggctttcgggtagcggccgaggtgggcgcgacggcgggatcgggatttcgggaagcggccgaggtgggcgcgacggcgggatcgggctttcgggaagcggccgaggtgggcgcgacggcgggatcgggctttcgggaagcggccgaggtatataattcagttatacttacgggtgtgggttgagattcagtcgataattgtatacacgcgatcgttaaaattaaatttatttgtatccgcttcatcctggtttttattgacacatccacgctccccttaaataaaagattaaaacacccaccgcggaatttaaatgatggaaacgtttttttcgacattgggaataataaaaacggcttttgcaactttaaaaactttatttatctcgattacgatacatatgtaccacatagatagcatagatttattataatataaacacacacgtgatatactttagtgatatgagatgccataaaacagtcaataggtttaacgcttaatctcatcatctgaatacacgtcaaacccgccgcaactgttgatgttagaattataatagctccccatgaaatgccggcaaatgttacagctatacccgtcaccgaggtcgttgtatataatacaattacccataggtttttttttcttgatataaaacggcaaaaccctgtaacccaaatgctataatatgacctcctattgaaactgctaacgttacttgtgtaagtttgataaaatgatttaatttaattatatgtgagattgcccacattaatggggtaactatatataacaccgggggtataacagacattatacgaattcctttaaacacgcgtttaagggtccgggaactttctctatggtcacatactctcccgcggtcattttgtgtatatacaacggcaaaacctaaatctgtataagtgtttaattgcttatggcgatttttacgatatatacacgtatcttgcaaatcggtggcggcatcgacaattgaaactagtgtgacaatagatatacacaatccaataagaacctcatatttactgacatacatatataaaataacggttagtaaacctcccaacccagttcccaacatcataacataaaaataaatatgcggtccattgaatgtcgtaacaaagttgtagtaatggatatgcacagcagccactgttccggtaatcgcggatatggaaattcccagtaattctacaaatggaagatcccgggatattgggcaaccaaccgcccataacacagcaaaacccaacacgaccaccgtctgcaaacatcgtcccaattttgctaatgtgcgtagaaatttcacggatgttggccataaccccgaaacgacgatcaaccccataatagttgcattgacggcagcttcgcagacgtgatattgtaaaattaacccggacgtgataacacttgcttgtagtcccacgagaaacaaccgcgatgctgaggttattgcacacgaattacattcttgagggtttccgacacatccttggattgattgagcgcggattaattctctgtctaacacacccaggttttcatcatggacagctctttcaccattcacggccatgtcttaagtttaataattcaaaacagataaaaatgtgttcatctatggtacacacaagtttgtatgtaaaatataagcaaaagttgcacttatttaactgtacatattacgtcagattcacgtgataattcagaataatccagggttcctgcagggtccactggaggagccacacaatattcgcgaattccgattccctcctgccatgtggtttcggggagtttcccccccattttatttccggtatttttttcgtttctttttgttaataaattgcgtcttttttttaatggtggttcatccttcacagattccgtgttcgcaaataattgcatcgaggttaatttttctttaaggtctttgggacttaagaacgttgcataaaaaaaagaatgcacgggtgcggaacgttggatatacaatccaaccatgggggagttagttaaggcgagataaaaattaatataacacgtctcatcccgtgttaacttaagattttgtacggcagaacggaatccactgtgtgtttccaataatactccaaattcacgcatactcccgctgccataaacaacattattaaggatcctttttgaatttgtgattgagcgtattaaattatatggtgtaggcttgcttccgtttatatccaaggaaacattaaatgagataaaaccacccccggcggtctggatgtacatatccgtggctgttagaatgaagcatgttgtaaacccaaaagttttaagtagtcgctgtaaacgggtgaattgatcgcgttttaagcaaatgcttatatctggagttagatttgggaacatcattgtataacaagcgagttcacgttttacaacttgtttgtaacattgtacttgatcatctggaccacaatcacccgggcgttgccataccatcgtttggataatactccgctcggggggttgtccggtaaatttaaaatataaccgtgttggggtcgacggatcttttgtatggcgaaacgcgtcaataagcgaggaccgtccctccgttgccgcgagtacaaccattctcggcccagtccaattatactggtcaaacatatttgccggtataggaatatacagttgttctgtttccaaactacagtgaataattaatccttcgtcgctgaatattaaaatagaatcccttagtctattaaccagaggtgatatagacgaaattaaaccagtaagcgttttttccgttaaaacagctctggcgatttctggggcgtcaaaacccgcatgcaattccatgtccaaagcatcgtctgtacgcgacctcaaatccataatttactacttaaaatgtttactatagaaaaagtaatcatatgtaaacacacgagtttcgttaatatgtttgtttaacccgatccggtgacttaagtacataaacaggcatgatatttgaatagtacggcccatgggagggaacatttccacgtgttccaatacagggggtgttccttaatagggactgtgcaataaaatacgtaagaagttaccagatttgatgtaatgtttgtcataaaaaatatgtacatcattatatacgtctgtaattaacacaagatcacatcgaagaattactgaagccgctgtgaaacctttcacaagacgatataaacttggttaagtgtattgatggggctctttggactgacacgctttatccatgaacataaactggttaaacccagcatcatttcaacgccacccggagttttaacccccgtggcggtagacgtatggaacgtcatgtacacattgttggaacgtttataccctgtgggtaaacgcgagaatttacacggaccatctgtaacgatacattgtcttggagtcttattgcggctattaacacaacggtcatactatccgatatttgtattggaacgttgtacagacggcccattatcacgtggagccaaggcaattatgtcacgggccatgaaccacgatgaaaggggaacctcggacttaacccgtgttctactatcatccaacacatgttctatcaagtataacaaaacatcggaaacatatgacagtgtgtttcgaaactcttccgcgagttgtattcctagcgaagaaaacaaatcccaggatatgtttttggacggttgtccacgacaaactgacaagatgatctgcctgcgcgaccaaaacgtatgcagtcttacctctacaatgccatcccgaggacatcctaaccatcgattatatcacaaattgtgtgcaagtcttattagatggatggggtatgcatacgtcgaggcggttgacattgaggcggacgaggcatgtgcaaacttatttcatacgcgtacagtggctttggtttatacgacagatactgatttactcttcatgggctgtgatattttgttagatgcaattcctatgtttgctccagtagtacgatgtcgcgatttgcttcaatatttaggaattacataccctgaatttttggttgcctttgttcgctgtcagaccgatttgcatacaagtgacaacctaaaatctgttcagcaagttattcaggataccggcctgaaaattccacatcaaatggacacttcaacgcgctcccccacttacgactcgtggagacatggcgaggttttcaaaagtcttaccgtagccacgtcgggtaaaacagaaaacggagtgtccgtttccaaatatgcatctaaccgatcggaggtgacagtagacgccagttgggctttaaaccttctgccaccctcatcctccccattggataatttggaacgcgcatttgttgaacatataatcgccgtggtaactccattgacccgcggtcgcctaaagttaatgaaacgtgtaaatattatgcaaaatacggcagacccatatatggttattaacaccttatatcataacttaaagggggaaaaaatggctcgccaatacgcacgtatttttaaacagtttattcctactccactcccactaaacactgtattaacaaaatattggaattaaaacacacataagagcgacttaatggttcattgttttattttgctcgtatatacatgttataaatcgtttatcactgtgcccgcataagatgtactgtgtctctcaaaaaaatttgtgtttttatctgcaatcataaatgcaagtggaaagtccgaatcgggaggtggggtgttaaatagttttggtacattaatcgctgataaaagcctgtccgcgctgaatttcacgtattgtgtaattgcatcgacgttcaccaaacgggttttgggtgcatgggattttaaaaacgcacactcgatttcaacggcttccgaaaacagttgatgtattctggtgatagcgggtttttcgggtacatagttattgtatatacaacacgatgcgctggtatgtatggcttcatctcggcttataaggtcgttaaattgacaagttacaacaaatagtccgttattgcgtaaatatgcaatagccgcgaacgatgatacaaaaaaaatgccctctataagaatcattagtatatatttttctgcaacggatgggttgtcccgtaccttttcttccaaccattgtactttttgttggatcgacggattattaatagtgacgtttacgtattgtacccgcaacgattcatcccctctgaacaacattagttgaatttgactatagacacgcgcgtggacaacctcgatgcactcttgttcaatgtagtaatggtgaatatccttttgggaaaagagttgggttagagagcccaaattaacatttaccagatcatctgccgccgataaaaatgtaaaaataaatctgtagaatattagttcatcttccgttaaacagtccaagtattgataatcatcttcaatgataaaatcgctttctaaccaacgattcgaaatgctcagggcacgtaaattgtttatatctggacactccggcctgtaaaaaaaatgactgcaatctttctgatccattttggaatagtttcccgtgtaaatttataaagcacaactggtacaggttaattcgcctcccgcaaacagtccgctgttcgtagctttacgaattttacagtagtacatacccgttttaaggccggctttataggcacgtataagcaaattcattattttggaggcgggaattgtcccgtctgggcgttcctcaataaataaagtcattgattgactttggtcaataaatggcgccctttctgcacacatatcaacgagatcctcttgctcatattcaaacgctgttttatattttaagagtgggtgactattagataaacagccaaacgaacgtattactgaccattggtttttctcaagtatgtttataacttccagtcgtttttcttcacatgaatacatatctcttagttcgtccataaggtctaagttgggtctaagtaactcacccgaggtggtgaccttactaaacatattattataaattggagagaaaccctcactgcactccgttacctgtgcagatgaaactgtgggcattaacgctaagaactgcgagttgtataacccataagcgcaaatatcatctcgcagggtacaccatggtaaatctaaataacttatcgtagaaaacccatcttggtgtaaccatcccttagcatatttactttcggtaaaacccttaaacggggctaagccgccaatcttacacatttccatgcttgttttcattgtctcatacaacattaactccgctatttgtacatttaaccgtctagctggttgggaagttaaatcaaatcctaagcggagacaagttgtatgtaacccttgtatgccaatgccaagtgatcggttgttttttacacctttacatgattttttacatggaaagttcccagccgccaggaccccgtttaaaaaaataacagtcgttcttgctgtcaattgaaggtcgtttaaattaaatgacactgggcctttggataagcacgttgtaagatttatgctggcaagattacatacgccgtgttgatgagcgtctgccttttgaacaatttccgtacacaaatttgaccccgtgatagcatttccttgggtattcatatgataattacgattacaggcatctttgaacattaaaaaggggcttcctgttacagcagcactgcgtatgattgtgaatgcgatatcttgaatgggaacagaagaaacgcctaatccttctctctctaaacgtaaataggttgaagtgaatgcctccccgtgtaatgttcgaaggatatcggctctgttatcaaaaagagtccactgaacattactagccccttttagatagcttaggtatctttcaaaaaataaatctggggtccataaacaacaaaatatgttatcacatcgaaatatttcatcacgaaccaacattccacgtgtggccaaaacagtttgtagatcgacgtgccatggttctatgtaaacacaaactccagttggtcgttcacaatcactgttaattgccataaccatgcaatctaaaagttttaaaactgcaagaagacctttcgtttgattttccgtaggtattaaattcagactctgtagagaaattcccactccacctcgactttgtaataccgttcccacatcgcctgtgatagctcgaacagctctcccaacagtgatggattccgggtccattaaataacaactggccgttgccccggtctctcgacctaaaaacatcataaccggtgtagccgggacaattttctgacatgccaacgctgtgaaaaatacccgacagacatcagtccatgtataaccatcatttattccgggaataagagttgcgattttaggcaggtttacgatttctgttgtcacggtggccgccagtcttaaaaagaattggcaaagcgactctaatttaccttcctctaacttagttaaataaaagtcttcgtactttaaagcagactgtagtccaagggtagctaaagcggggtattgatctttcaaaaacggttctaatatagcccgacgaatttcgtccctccgcccttcaattgcttggcggactcggggagttaaacagagaattggggaagtcaaccacgtttccatggaaacggatcgtaggttaatacggcaatggataagttctccacaacatcggtacactcgctcatcttgtcgcgtcaccgccttaagttttgagacgatagtgctaatatactccattaattccaccggtgtggttgattcgggcggaatgatgtattccttgtagccatgttgacataatcggtttataatgtcatgaaccgtattaaaaattcttttgaactccataacggataacgtatttaggctccggaataaacctttaaaccctaaactcacagctgagttagttctacaatattgtagactcccttatatatggttacgtacagcctgcccctccccagtatataatatcacgcaaaacccacgctatgttaaattcagtttattttacatacatgctttaataataacattcgttccatgtatttgtacccccccacacaaccccctctaaccaaatagttggcacgttataacctccgaaccgttccatgcgtcttgtataacgcacagactctgatggaattgttccaattaacgtatatgccgcatacatgcaggataattgtgtgggaaggccccgaaaatcgccggtccattgatacaatcgctgtctagccaagttccaatttactcctgtaatttcgccaatactacatcgagggcttgtcgggtcattggataactgcacaagcggcaacgcccttgtgttatatggctggtgggtatttgcaaccccttcagtcccccaggcggcattttcagctcgtatgcgtcctaacaggaagccaataccacgaccaaaacattgttcgtttagttggcttaatgcaagatgcagtcttacaccttctcgttggcgtcgctgtgtatatacaaaaaccaagaacacatgcttcagtccgtccgcggaaagatgtaaatctttgtcaacgtcccaaaatacgcaggccgggatgttggctgtgaccctgcgagttgaagttttgtctgtacgtgcagcttcttggggacctttggccacggcggttatattgcataaattatcctgaatggtatattccagcagggacccaaaaaaacttataaatcgatgtggaaatacatgacattgtaccatcgcacgtaaacactccgaaaaccttatgagccgcgtttccatacgactgcatccataggcagaaacaattgctgttctgttggcatccgctgcctgtttatccgtatattcttctgcccggcatgcggcgatgaaacttaatgacgttacatatgctctaagccccccaccttctccaacggtccaaggagccgtgcaggcattgaataggtttcgtaaaccctctagtagtacatcggggtcacgtccagcctgtgtaagtgtattagcttctccaatcatgtcagatggatgacgaaggattaagacgattgacccagcatgctcaatgtccggacgaaaaaaatcggttaatgacacttgttggattagctgtgtcgttgatttaaaattatttaacgggagtctaatggtaacttgcgggttaccaattgaagttggatttatttgaatgttgttcatacgattaataacaattgaacggggggttacttgaatagacgcggtttctgtacgttttggtggtacatgtatcggttgtttgttcagacctccaaagcgagggccaattgttaaatcgcgactccaatttccgaagaagcccggagcataagtcatatgaagcccgttccctatttgaataaaacggttatttcctaaaagactgatattagttccacatagcgtttgttcgtttaaagtaaaatgcgagttggttggttgactccccatagctgaggggttaaattcacacaatgcaatcgtgacgtggtactatctgaaatgttgcctggggtatgtgtacacattatacagtcgtagtaccgtttatataatgttaggtaggaggagcctataaaaatattttgattggcgttaaaaggttcttcaacttaccgtgacgtcctttttattaacatgcgtttttattgatgttacatttatgtcttttcattccggacggatgtagctttttcatatcacgttataaagttaagtcagcgtagaatataccatggaagaaccaatttgttatgatacacaaaaacttttggatgatttaagtaacttgaaagtacaagaagcggacaacgaaagaccatggtcaccagagaaaacagaaatcgccagagttaaggtagttaagtttttacgatctacccagaaaattccagctaaacattttattcagatatgggaacccctgcattctaatatctgttttgtatattccaatacatttttggcggaggctgctttcacggccgaaaatttacccggactgttgttttggagactagatctagactggacgatagaggagccaggtaatagcttaaaaattttaacccagctatcaagtgtagtacaagattccgagacgttacatcgtttatcggccaataaattacgaacctcgtctaaatttggacccgtttcgatacacttcattataacggactggataaatatgtacgaggtcgccttaaaggatgcaacaacagccattgaatcaccattcactcacgctcgtattggaatgttggaaagcgccattgcagctttaacacaacataaatttgcgatcatttacgatatgccatttgttcaagaggggattcgtgttttaacacaatatgcaggatggcttcttccgtttaatgttatgtggaatcagattcaaaatagctcactcactcctctaacacgagccctttttataatctgtatgattgatgaatatctcacggaaacgccagtacatagcatatcagaattatttgcagatactgtaaatttaattaaagatgaggcgttcgtatccatcgaagaagcggtaacgaatccacgaacggtgcacgagtcacgaatttcctcagctctggcttatcgagacccttatgtttttgagacatccccgggaatgcttgctaggagacttagattagacaatggtatatgggaaagcaacctcttatcgttgtccacccccggaattcatattgaggcgctgttacatttactaaactccgacccggaagcggaaaccacatctggaagtaatgtagcagaacacacccgtggcatttgggaaaaggttcaggctagtacatcgcctagtatgttaataagcacccttgccgaatccgggtttacaagattttcatgcaaattgctacgtcggtttattgctcaccacacactcgccggttttattcacggaagcgttgtagcagacgagcatattacagatttccaacaaacactaggatgtctcgctttagtgggtggactggcataccaattagtggaaacgtacgctcctactaccgagtatgtgttaacatatacacggacagtaaacgagaccgaaaaacggtatgaaacgctattacccgccttaggattaccaccgggaggcctgggacaaattatgcggcgctgttttgctccacgaccccttattgaaagtatacaagcgacacgcgtaatactacttaatgaaatttcacatgcagaagctagagaggcaacatattttaagcaaacacataatcaatcctcaggtgcgttattaccacaagcaggacaaagtgccgtacgcgaagccgtactaacctggtttgacctacgtatggattcaagatggggtattactcccccggtggatgtgggtatgacacctcctatttgtgttgatccaccggctacagggttggaagctgtcatgataacagaagcactaaagattgcatatcctaccgaatataatcgctctagcgtgtttgtggaaccgtcgtttgtgccttatattattgcaacaagcacgcttgatgccctttcggcaacaatagctttgtcttttgatacacggggaatacagcaagccttgtctattcttcagtgggctcgcgattatggatccggaaccgtgcccaatgcagatggatatcgcacaaaactatctgctcttataacaatattagaaccttttacccgtacacaccccccagtacttttaccatctcacgtttctactatagattcccttatatgcgaacttcatcggactgttggcattgccgttgacctgcttccccagcacgtccgtcctttggttcctgaccgtccttctattacaaatagcgtttttttagcaactctctattatgatgaactttacggtcgttggacccgactggataaaacatcgcaggcgttggttgaaaattttacatccaacgcgttagtggtttctcggtacatgttaatgttacaaaaattttttgcgtgtcgtttttatccaacgccagatcttcaggctgttggtatctgtaacccaaaggttgaacgcgatgaacaatttggggtatggcgtttaaacgatcttgctgatgcggttggtcatattgttgggacaatacaaggaatccgaacgcaaatgagagtgggaatatccagcctgcgcacaattatggccgatgcttcctcagcccttagggaatgtgaaaatttaatgactaaaacctccacttctgctattgggcctcttttttcaacgatggcttcccggtatgcacggtttacacaggatcaaatggacattttaatgcgtgttgacaaactaacaacaggagaaaatatacccggtcttgcaaatgtagagatttttttaaataggtgggaacgaatagcaacagcttgtaggcatgccacggcagtcccgtcggccgaatctattgcaaccgtgtgtaatgaattgaggcgcggtttaaaaaatatacaagaggatcgtgtaaatgccccaacctcatatatgagtcacgcccgaaatctggaagatcacaaggcagcagtttcattcgttatggactccaggcaacagtttattgtggattctggacctcagatgggcgcggttttaacttcacaatgtaatataggaacatgggagaatgtaaatgcaacgtttttacacgacaacgttaaaataacgacaacggtcagagacgtaatttcagaggctccgacgctgataataggacaaagatggcttcgtccagatgagattttatctaatgtagatttgcgtcttggcgtacccgggaatacaagtgggagtgacccttaatataaaacaggcgtgtttatgtacattaaagtatttgtggtttttattgactgggcgtttcgtttgtataacgctgttgttgctagtattttcataacctcctaggtttttggagctacacgtgcttattcaacgctctttgggatttgaatcatcgtaaacgtagcgtccctaccagttgagcgcgtaattttcgtaagcaataaaatggatataattccgcctatagctgtcactgttgcgggagtgggaagccgtaatcaatttgacggtgccctgggaccggcgtcaggtctgtcatgtttaagaacatctttatcgtttttgcatatgacatatgcgcatggaattaatgcaaccctgtcatcagacatgattgatggatgtttacaagagggtgcagcatggactacggatctgtctaatatggggaggggtgtcccagatatgtgtgctcttgttgatctccccaatcgaatttcatatattaaactgggggacactaccagtacgtgctgcgttttgtctagaatatacggcgatagccatttttttaccgttccagacgagggttttatgtgcacacaaattcccgctagagcgtttttcgatgatgtgtggatgggacgtgaagagtcgtatacaattataactgtagactcaacgggaatggccatctatcgtcagggaaacatatcttttatttttgatccacatggccatgggactataggacaggctgtagttgttcgggtgaataccacggatgtgtactcttatatcgcatcggagtatacccaccgccccgataacgtagaatcccaatgggccgctgcattagttttttttgtcaccgcaaacgacggtcccgtaagcgaagaagcgctatcttcggcagtaacgcttatatacggaagctgtgatacatattttacagatgaacaatattgcgaaaaactggttacagctcaacatccgttgcttctttcacctcctaattccacgacaattgtgcttaataaatcgtctatagtacctcttcaccaaaacgttggtgaaagtgtatccttggaagcaaccctacattcaacgttaaccaacacggttgcactggaccctagatgtagttacagcgaggttgatccttggcatgcggttctagaaacaacctcgactgggtctggcgttttggattgtcgtcgtagacgccgtccttcatggactcctccttcaagcgaggaaaatttagcttgtatcgacgatggcttggtaaataatacacattccacggataatttacataaacccgctaaaaaggttctcaaatttaaaccaactgtagacgtgccggataaaacacaagtggcacatgtattaccccgcctacgagaagttgctaacaccccagacgttgtgttaaatgtatccaatgtagatacgcctgaatccagtcccactttttcacggaacatgaatgtaggaagcagtttgaaagatcggaagccatttctatttgaacagagtggtgatgtcaacatggttgtcgaaaaactactacaacatgggcatgaaattagcaatggatacgtacaaaatgcggtgggtacgttggatactgttattaccggtcatacaaatgttcccatttgggtaacaaggcccttggtcatgccagacgaaaaggatccattggagctttttattaacctcaccattttgcgtttaacgggatttgtggtggaaaatggaacacgtacacatcatggtgctacaagcgttgtatcagactttataggtccccttggggaaattttaacaggatttccctccgccgcggaacttatacgcgttacaagtttgatattaacaaacatgccgggggcggaatatgctattaaaactgttctccggaaaaaatgtacaattggcatgctcattatcgctaagtttggtctagttgccatgcgggttcaggatacaaccggcgctttacatgccgaactagatgtgttagaagcggatctaggaggttcgtcgcccatagacctctattctagactgtcgacaggtcttataagtatactaaattcgcctattatttctcatcccggactttttgccgagcttattccaacccgtacagggtccctgtctgaacgaatacgtcttctttgtgaattagtctcggcccgggagacacgctatatgcgtgaacacaccgcgcttgtttctagtgtaaaggctttagagaatgcattacggtctacccgcaataaaattgatgccattcaaataccagaagttccccaggaacccccggaagaaaccgacattccacccgaagagttaattcggcgtgtatatgagatacgatccgaagttacaatgctattgacctcggctgttacagaatacttcacccgcggagtgttatatagcacacgggccttgatcgctgaacaatcccctaggcgttttcgggtcgcgaccgcaagtacggcacccattcaacggcttttagattctcttccggaattcgacgctaaattaacggcaatcatatcgtccctgtctatacaccctcctcctgagactatacaaaatctccccgtcgtatctctgttaaaagagcttattaaagaaggggaagatttaaacacagacacggctctcgtatcgtggttatctgtagtcggggaagctcaaaccgcaggttacttatccagacgagagttcgatgaattatcacgtacaattaaaaccattaatacacgcgcaacgcaacgggcttccgcggaagcagagttgtcttgctttaatacgctaagcgcggccgtagaccaagccgtaaaggactatgaaacatataacaatggtgaggtcaagtatcctgaaataacacgggatgatttattagcaacaattgtacgtgctacagacgatttggtgcgacagataaaaattttaagtgatccaatgatccaatccggtttacaaccttcgattaaaagacgattggaaacaaggcttaaagaggttcagacgtatgcaaacgaggcccgaaccacacaggacacaataaagagtcgaaaacaggcggcatataataaactcggggggttacttcgcccggtaaccggttttgtgggacttagggctgcagtagatttattaccggaacttgcttctgagttagatgtccaaggagccctggtaaatctcaggaccaaagtcttagaggcgccggtagagatccgttctcaacttacgggtgatttctgggcgttatttaaccaatatcgagacattttagaacatcccggaaacgcacgcacatctgtcttaggaggactgggagcttgttttacagctattatcgaaattgtgccgatacctacggagtatagaccatcattgcttgcgttttttggtgacgtggcagatgtgcttgcatccgacatcgcgaccgtatctactaacccggaaagtgagtccgccataaacgctgttgttgcaactcttagtaaagcgacgttagtttcatctacagtgccagccttatcctttgtgttgtcgttatataaaaaatatcaggctttacaacaagaaattacgaatacccataagttgactgaattacaaaaacaacttggagatgacttctccaccctagctgtctcatctggacacttgaagtttatatcatcttcaaatgtagatgattatgaaataaacgatgcgatattatcaatacaaacaaatgtgcacgccctgatggatacggttaaacttgttgaagttgaactgcaaaagctacccccccattgtattgctgggacatctaccttatctcgagtagtaaaggatcttcataaactcgtcacaatggcacatgagaagaaggaacaggcaaaagtgttaattaccgattgtgaacgtgcacataaacaacaaacgactcgggttttgtatgagcgttggacacgtgatattatagcatgtctggaggcaatggaaacgcgccatgtatttaacgggacagaactggcacggttgcgagatatggccgctgcgggagggtttgatatacacgcagtttacccacaagcacgtcaggttgtagcggcatgtgaaactacagccgttacggcattagataccgtgtttcgccacaatccacatacccccgaaaatacaaatattcccccacctttggctttgttaagagggttaacatggtttgatgatttttcgattacggctcccgtattcaccgttatgtttccaggtgttagtattgaaggactccttctgcttatgcgtattcgcgcggttgtgttattatccgccgatacgtctattaatggaatacctaactaccgagatatgatattacgaacctcgggggatctattacaaatacccgcattggctgggtatgttgatttttacacacggtcttatgatcagtttataaccgaaagtgtaacgttaagtgaacttagagcagacatcagacaggctgccggggctaaacttacagaagcaaataaggctttggaggaagtaactcatgttcgggcacacgaaacggctaaacttgcacttaaagaaggtgtcttcattacattaccaagcgaaggtttattgattcgggctatagagtattttacaactttcgatcataaacgatttataggaacggcatatgaaagagttttacaaacaatggtagaccgcgatctaaaggaggccaacgcagagcttgcacagtttcgtatggtgtgtcaggcaacaaagaaccgtgcaatacaaattttacaaaacattgttgatactgctaatgccactgagcaacaagaagacgtggatttcactaacctgaagacgttattaaaactaaccccccctcccaaaacaattgcattggccattgatagatctacttccgttcaggacattgtcacgcagtttgcattgctgttagggcgtctggaagaagaaactggtacgttggacattcaggcggttgactggatgtaccaagctcgcaatattattgactcccatccactaagtgtgcgtatagacggtaccggccccctgcatacttataaagatagggtggataaactttatgcgttacggactaaattagatctcctacgacgacgaatagaaaccggtgaggttacgtgggacgatgcatggacaacatttaaaagagaaacgggggatatgttggcatcgggggacacgtacgctacttccgtagatagtataaaggcactccaggcatcggcgtctgtggttgacatgctttgttccgaacccgaattttttttattgcctgtggaaacgaaaaaccgtctccaaaaaaagcaacaagaacgtaaaacggcgttggatgttgtgttgcaaaaacaaagacagtttgaagagaccgcgtctcgcttacgagctttaattgaacgtattccaacggagagtgaccatgacgttcttcgtatgttattacatgatttcgatcaatttacacatttgcctatatggataaaaacacagtatatgacatttcgaaatttactcatggtacggttaggcttgtatgcaagttatgctgagatttttccacccgcgtctccaaacggagtgtttgctcctattcccgccatgtcgggtgtatgtctagaagaccaatcccgatgcattcgcgcgcgggtggccgcgtttatgggggaggcgtctgtggtgcaaacgtttagggaagccagatcttctatagacgctttgtttggaaaaaatttaaccttttacttggatactgatggggttccacttcgatatagagtgtgttataaatcagttggggttaaacttggaaccatgctatgcagtcagggtggattatctttacgaccggcacttcccgatgaaggtattgtggaagaaactacactatcggcattacgcgtggccaatgaggtcaatgagctacgcattgaatacgaatccgctataaaatccgggttttctgccttttccacctttgttaggcatcgccacgccgaatggggtaaaaccaacgcacgcagagccattgcagagatatacgccggccttataacaacaacattgacacgacaatacggggttcattgggacaagcttatttattcttttgaaaaacaccacctaacttctgtaatgggcaatggactaactaaaccaatccagagaaggggtgatgtacgcgtattagagttaaccctatctgatattgtaactattttggttgccacaaccccggtacatcttctcaattttgctagattggatttaattaaacagcatgagtatatggcccgtaccctcagacccgtaatcgaggccgcatttagaggtcgtttactcgttcgctcattggatggagacccgaaaggcaatgcccgggccttttttaatgccgccccatccaaacataaactcccgttagctcttggatcaaaccaggatcctaccggcgggagaatatttgcatttcggatggcagattggaaacttgttaaaatgccacagaaaataacggatccttttgcgccatggcaactttcccccccccccggggtaaaggccaatgtcgatgcagttacccgtataatggcaacagatcgtcttgcgaccattactgtacttgggcgcatgtgtctcccgccaatttccttagtgtcaatgtggaatacgctgcaaccggaggaattcgcatacagaacacaagatgatgtggacattatagttgatgcgagactggatttgtcatccacgcttaatgcaagatttgataccgctcccagcaataccacgttagagtggaatacagaccgtaaagtaattacagatgcttatattcaaaccggggcaacgacagtttttacagtaacgggggcggcaccaactcacgtttctaatgtaacagcgtttgacatagcaactacggctattttatttggggctcctttggttattgccatggaacttacatccgttttttcacaaaattccggacttactttggggttaaaattattcgattcccggcatatggctacagattcgggtatatcctcagccgtatctcccgatattgtttcttgggggttacgtttactgcatatggatccccacccaattgaaaatgcatgtttaattgtccaactagaaaaactgtccgcgctcattgcaaacaaacctcttacaaacaatcccccgtgtttactgctattggacgaacatatgaatccctcttatgttttatgggaacgaaaagactcgattccagctccggattatgtggtcttttgggggccagaatctcttattgatttgccgtacatcgactccgatgaggactctttcccctcgtgtcccgatgatccattttactcgcaaattattgccggttatgcgccccaaggccccccaaacctcgacacaactgatttttacccaacggagccactatttaagtctcccgttcaagttgttagaagttccaaatgtaaaaaaatgcccgtccagcccgtccagcccgcgcagcccgtccagcccgcgcagcccgcgcagcccgcgcagcccgtccagcccgcgcagcccgtccagcccgcgcagcccatagaaccgggcacacaaatagtggtacaaaattttaagaaaccccaaagcgtaaaaacaacccttagccaaaaagatattcccttgtatgtggaaactgaatcagaaacggctgtgcttatacctaagcaattaaccacctccattaaaacaaccgtttgtaaaagtattaccccaccaaataaccaattgtcggattggaaaaataatccacagcaaaaccaaacgttaaaccaagcgttcaataaaccaatacttgagattacctccattccgacagatgactcgatatcttaccggacttggattgaaaaatcaaatcaaacacaaaaacggcatcaaaatgaccctcgaatgtataactccaaaacagtattccaccctgtaaataaccaattaccttcttgggttgacacggcagccgatgccccccaaacggacctattgacaaactataaaacaagacagccgtcgccaaactttccgcgggacgtacacacatggggcgtatcttctaacccgtttaactcaccgaacagagacctatatgaaagtgattttagtgaaccttctgacggctatagcagtgagagtgaaaattctatcgtactaagtctcgacgaacatcggtcatgtcgcgttcctaggcacgtacgtgttgttaatgccgatgtagtcaccggtcgacgttatgtccgagggaccgccttgggagcactggccctgttaagccaggcatgtcggcgtatgatcgacaacgttagatatacacgtaaacttttaatggaccacacggaagatatatttcaaggcctggggtatgttaaattgttattagatggaacatatatataaagtagcgcctattaaagaaaaaaaaaacaacgattattttctgtgtatttttatttacaccctacgacttcttgaagcgtttccagattgtcccgtgtgggacaaggtctgtcccttacccccctggggggtattttgggttgggggcggggtagactgtggcacgccttgggccgcgggcggtgatccggttgttggctggacagtgcttgactgtgctccctgttgcggttgttgtccagaagaccccgacaccacgtgttgctgttgtccaacggatgccgacgtcgtttgaggtggggggtgttgcggggatgatcccgaaaacgccaacgcggcgggctgttgtaaagcagactgatcggcgctctgtgttttttgcggcaatatagtaggccccgagattcccaaactcatggatggatttgggggttgtggtcgtataatacgcgggttaaacgtacgttttaagccaaccgttggtcttaaccatgtcatagggtcagtctcggcaaacatggccgttcggcgtatcgtatttgcattatggttagcgcgtgcacgcgcggcactggccgcggctcccacggtgtaaatgcttctggcatcagcgatgtccacacggtgaccaggttgcaaaggtccactggcgtttaaaagtcgtattaaagcaacgggggtgtaagccgcaattgcttccaccgaaaatgtggtggggttgctgggatcaaagactacacgagacgatgcgggttgtgtcatcgtttattagtttacgggacaatcgataacagcatacacgtacatctgcgcaggatatgtacggaaaggcaatttatttccagaaaagcaccgcccctaatacaactaccagtacaattacaacgaacagggcatatgtcacgttagctacgggtagagcaagtttccagacacgcgtagtttgggtatcgggtaacgcaggtttaatgtcactttgcatttgaacagacgtgtttggacttccgttctcgggtggggatctgaatgaaggccgccagcgtatatattcatccaaattattgccagtttccttatacatgtatgcatccgtggcgcgggccataagtttaatggtgcgagatggatcttccggtcccataaaacgaaaggataactgaacatatggcattcgcacaaagcagttcacccacattaaagcctggagaggtcggcggtcaatacccccacctcgtttaattgattccaaagcagataggttgataccggtacttaacgttgaactaagaatcacgttattactgtcaatggacacttcagccactggtgcgttagtcggacgaaaaaaaaaaccttgaaatagcacagacacccccgtattttgaatttttatgtaagggtcacaatctacttgcgcccaattcgccattaaacgcataatatactctaccggaaaggcttcggatacgttgtcttcgccgttaaactgaaaaacacaacgggcgggggggcgttgtggatcaaatattggaagatccccatcgcaacattgaagagcgcttggtaccaccaaccgaatacgttgtaaaagattatctccgcaacccctcctgcgttcactccgtacatacgttctccgtgacatattgatctaaggttgcaaaccaaggcacacgcgtgaagtatttagaccatttatcgtgggatataggaggagtttggagtgatccaccccctgacgacttattaatgcgtttattttccccatgtattaagcatccttcaatatttcatgcaaatctagaaatttggccatgactcccgcaaagcgttcacggcgacgggtcacgctggcactatgttcacatggaacaacataagcagatttttctgaatcgttactttctttatgttttaaaacggacgccaggcgactggtaaatgatatataatttaattgagcgtcagttgtaggtagaattgcttctatttccgggggaattaaattttcaaaccaaacggaaagagtaaaggtgctatcagcaggaaaatactttgactccagtgcatcgatatttaatagattaacatcggtgtctgtaattaaatcgcgggccctcatcccagagatggatcgggtagaatcagaagaacccatggatggattcgaatcgcccgtattctccgaaaatacatcttctaattccggatggtgttccgacgcattttccgattcgtacatcgcttataatccagcccttctgctaaaaaacgatttgttattttcagaattgttatttgcctcccacttaataaatgttccccgtgcaatagaaaacaacgtcacttatgaggcctcttcggcggtaggtgtggataatgaaatgacctcaagtaccactgaatttatagaagaaattggagacgttttggcgttagacagagcctgtttggtctgtagaacgcttgatttgtataaacgtaaatttggactgacaccggaatgggttgcggactacgccatgttatgtatgaaaagtctggcatccccgccctgtgcagttgtcacttttagcgctgcctttgaatttgtgtatcttatggatcgttactacctgtgccgttataacgttactttggttgggtcctttgccaggcgcacgctttccctgttagatatacaaagacatttttttttgcatgtatgttttcgtaccgatggagggttaccaggtatacgaccgccccccggtaaggaaatggccaacaaagtaagatattccaattactccttttttgtacaggcggtagttagggctgcattactatcgatcagcacgtctcgtttagacgaaaccgaaacgcgtaagtcattttactttaatcaggacggactgactggaggccctcaacctttagcggccgccttggctaattggaaagattgcgcgcggatggttgactgttcatcatcggaacatcgcacaagtgggatgattacctgcgcggaacgtgcattaaaagaggatatagagtttgaagatatattaatagacaaacttaaaaaatcgtcttacgtagaagcagcttggggttacgcagacttggctttattattactgagtggggttgctacttggaatgtagacgagcgtacaaattgtgctatagaaactcgcgttggatgtgttaaatcatactggcaggcgaaccggattgaaaactccagggacgttccaaaacaattttccaaatttacgagcgaggatgcctgtcccgaagtagcatttgggcctattttgttaactaccttaaaaaacgcaaagtgccgtggtcgcacgaataccgaatgcatgttatgttgtttattaaccatagggcactattggatcgctttgcggcagtttaaaagggatatattagcatactcagcaaataacacaagtttatttgactgtatcgaacctgtaatcaatgcatggagcctagataaccccattaaacttaaatttccatttaatgatgagggtcgattcataaccattgtaaaagcagcaggttccgaggccgtatataaacatttattttgcgatctcctatgcgctctctcggaattacagacaaaccctaaaattttatttgcccatcctacaaccgcggataaggaagtgttggagttatataaagcccaactggctgcacaaaacagatttgaaggtcgtgtatgtgctggcctgtggacattggcgtatgcatttaaagcctaccagatttttccacgcaaaccaaccgccaatgccgcattcatacgagatggaggacttatgcttcgacgacatgcaatatcgctggtctccctcgaacacaccctatcgaagtatgtctaggcgatataaatccgtatctcggagcgggccttcgatgcgtgtacgctccagaacgccatgccgccgtcaaaccattcgaggaaaacttatgtcaaaggagcggtctgtgtaccgccattattttaattacatcgcaaggtcccccccagaagaactagctaccgttagaggcttaatcgtgccaattattaagacgacccctgtcacccttccgtttaacttgggtcagacagtggcggataactgcctgtcgttatccggaatgggttatcatttaggtctcggaggttattgtccgacatgcactgcatctggagaaccgcgtctatgtcgaaccgatcgggcggctctgatactagcatatgttcagcagcttaacaacatatacgaatatcgtgtgtttcttgcatccattttggcgctatcagaccgagccaacatgcaagcagcgtccgctgaacccctattgtcgagcgtattggcacaaccggaattattttttatgtatcatattatgagggaggggggcatgcgagatatacgcgtacttttttatcgtgatggagatgccggagggtttatgatgtatgttatatttccggggaaatctgttcacctccattacagactaatcgatcatatacaggccgcgtgtcgggggtataaaatagtcgcacacgtttggcagacaacatttttactgtcggtatgtcgcaacccagaacaacaaacagagactgtggtgccatccattggaacatcggacgtttactgtaaaatgtgtgaccttaactttgatggagaattgcttttggaatacaaaagactctacgcattatttgatgactttgttcctcctcggtgatttcagcttcagtgttcattttattatcccagcgcggggcgtgtatacaaacaaagcctgccgcctgcaagcggtttagcattttaacgttaacaactcgtgtctctggaataaaacgttttaaaagccgttctgtgagtttagtgtcgtttccaaataacgccttaaaagttacactcgccgtcccaatgagatgagaaaaataatagtcaatgtttaaagacagcccgtgtgatgttacgtgaatgggatcttccgctaagtcagatattattaacttacgctttgcttccccacaccgtttacctgcggtattctgtaaaggatctccacgtagcaaagctacactttttgcatcagcctccacttcgtctgtgggggccacaataacataagggatgcgttctcgaacgtttgggatttgaccctgtctcattactaatttataatatactgttaagtgagccaagcgacggtttatgtaggcggatggtggacgactaagctcggccgtcataacaaacttattaatatccaatttgggtgatgtaatctggcgatgtgcatctgcaattatgcgtccaaacccggccatcccagacggcatggcccgtctattccattcagcaatggaaacacacgacgcctccgccgcagcacgcgagacggtgtcgtcatataacaacagttctacaagtttgcgggcataatcgttaataaattgacagttgttttttctaaccaagtcgactcccttcattaaaacctttccgccgtaaattaccccaatgtactttttctttgttataagtaaaagttttataaaagttttttcacactccaactttataggaggacaaaacagagccgttgaaattatatgtgccattttctcgccgattttagctatcccctcaacaccaacacccttgaatcggataaacacagaatccgtatctccatatataacctttacctcgtacgctttttgggagagaacgctactttcaatgtctggaaacgctgtaataaaacgttcaaatgcggcccagttattatgaatataatctctggtacttaataacatttgacggccaattgtagtgacagtggccgctacgtataaacatggcagaaatccctgcgcaactccagtaaaaccgtacacggaattacaaactacttttatcgcggcttgttgtttgtctaataacactgcttcatctgaagaacttccgggtatgcgcgctctaatagccttgcgcatagccaaccagtcttttaaaagaacacccagcagactttctcgaacgttagagcgcacaaaaaaaagacgttttcctccaactgtaaaggtggcataatcggatggattcaaacgtttaaccgtctcaaaatttaacgttagcgtggtaaaacataagttatgggcctgaattatacttggatataaacttgcaaaatccaatacgaccaccggatcgatataaaatcccgtatcagggtcaaaaaccctggctcctttatatcctacatttcgcccacttgacgtaccagtgggagaaacgctctcgtcttcatccatctcttcctcaacatccccgacatcgggaataacatccttatattcaaaagtagctgggtatcccccatcgggtaaaataaatcctcgagacgaagccagtcctaataaacaagtgtaaatcctaacctgctgtccgtcgtaaatagccttggttaaagtaattctagctagccttgcaaccgcggataactcaaggtgtggtaaatatttaaaaaacagtttccccacaagagccgagtcttgtatacaatattcaccaataattcctcgtgtattcggtccactagcgtaatatcccggaatgtctttgtagggcaaatctctcttggactcatttagagcttcacgtgcaaccgaatctaatttataactcgagagttttaatttttcagttgcaattgcatacatatccagagatatgagaccgttgatctttaccttgcttcgtcgctgaaatccggatttgccaacatcccatatcttaaacagacccccacggtttatactgccataaccatcaagcttgagactgtatatagaattaagtttctccataataaacgcccaatcaaaattaacaatgttataacctgtggcaaactcgggagcgtactgttttacgagggtcataaatgcaattaatagctcgaattcactatcaaactccagcacagtcggctccggtaaccccgcgtccttcatttcttgtacatacctttgtggtaagtcacaagagccaagggaaaacagtaaaatgtgttctaaagactgtcgagggattgaatataatagacaagaaatttggattacaagatcctccaaatgtgttgcatcgggaaacgccagctcattagatcctcctgatttacattcaatatcgaaacataacaacttgtagtcaggccatgagtcatcgtttggtatagcctgcagattatccgacatgcagtcaatttcaacgtcgcttaacgttaattggcgacttgccggtcgaactcgaacacgttccccatcaactccaggttttagttgataccaaccaaaactaacaaagccgggattatccattagaaaacgagtggtagcgtctacccgaccttcatactttttcaactccgggtgaaagttatcacaaagataatttgtaaatttagatgagggagaatacaccctgtaaaacgcacatggctgtgtatcgtagtaataaacatctgtgcgctcaataacctcaacgcgaaagctttctggagatgcgcttttaaacgaggtaccatgaaaagcgttcttgtctccatttaacgttgcatcattttgtgttatcatagaactgcgtaaacactcggcaagtaatacagataactcgctacaggaacgtatgccacaagcggtatccacctcggctttgtttatataaaaatattgacagatgccgtatacatgaactgccaccctttttccacatcgggacatgccaagtaaagtaataacggtaccaagcggtcgtgttgcagttgcaaaccgggatacatctccattagacgcggcttctgttgtttcgacaatatcatatacatggaatgtgttaaagcgggggtcaaacttatccccacgaaagtcgatttccccccaaatattcacgcgtctaggccaggggctggaacaacgaaaatccagaatcggaacttcttttccattacagtaaactttaggcggtcgactaagtgtaccgacgtgaaccccctttcgttcttccatgggcacatcttcatctaaacatttaggggccaaaaattgaaacgatgacatggtagttttgtaactatgaagaaattctctgttactaccgcgcccggttcttgggttatatttaatccctgatgcttgggttaaaaagggattacaaaaccccgttctgatcgccattttatgttaacgattgataatcttgtaaaaagccagtgttactgagtaacacaaccccacgcccttctaatacataaagtgtaatcacgtgatttgttgtggtttccgcatatgtaatacccgtttaaaagcctctcttcttaatgtatcgacagactgggttttgggtggtcatttgaccctgccaacaaccccccattattacgagtacttcaccaaaatggaaaatactcagaagactgtgacagtgcccacggggcccctgggttacgtttatgcgtgccgggttgaagatttggatctggaggaaatttcatttttggccgctcgtagcacggactctgatttggctttattacctttgatgcgtaatttgaccgtggaaaaaacttttacatccagcctggcggtggtttctggagcacgcactacgggtcttgccggagctggtattaccttaaaactcactaccagtcatttctatccatctgtctttgtctttcacggaggcaaacacgttttacccagctctgcggccccaaatctcacacgcgcgtgtaacgcggctcgagaacggtttgggttttcacgctgccaagggcctcctgttgacggtgctgttgagacgaccggcgctgagatatgcacccgccttggattagagccagaaaatacaatattatacttggtggtcacggcattgtttaaggaagccgtatttatgtgcaacgtgtttctgcattatggaggactcgatattgttcatattaaccatggggatgttatacgtataccgttatttccggtacaacttttcatgcccgatgttaaccgtctggtacccgacccattcaacactcatcacaggtctatcggagagggttttgtatacccaacacccttttataacaccgggttgtgccatttaatacatgactgtgttattgctcccatggccgttgccttgcgcgtcagaaatgtaactgccgtcgcccgaggagcggcccaccttgcttttgatgaaaatcacgagggggcagtactcccccctgacattacgtacacgtattttcagtcctcttcaagtggaaccactaccgcccgtggagcgcgtcgaaacgatgtcaactccacgtctaagcctagcccatcgggggggtttgaaagacggttggcgtctattatggccgctgacacagccttgcacgcagaagttatattcaacactggaatttacgaagaaactccaacagatatcaaagaatggccaatgtttataggcatggagggcactttgccaaggctaaacgctctggggtcatataccgctcgtgtggccggggtcattggtgcgatggttttcagcccaaattctgcgttgtatctaactgaggtggaggatagcgggatgaccgaagccaaggatgggggaccgggtccatcatttaatcgattttaccagtttgccggacctcatttagctgcgaatccccaaacagatcgagatggccacgttctatccagtcagtctacgggttcatcaaacacagagtttagcgtggattatttggcactcatttgtggatttggagcacccctgttggcgcgactgcttttttatctagaacgctgtgacgctggtgcgtttacagggggtcacggggatgcgttaaaatatgttacggggacctttgactctgaaattccatgtagtttatgtgaaaaacacacgcggccggtatgcgctcacacaacagtacaccgacttagacaacgcatgccgcgatttggacaagccacccgtcaacctattggggtgtttggaacaatgaacagccaatatagcgactgcgatcctctaggaaactatgctccatatttaatccttcgaaaacccggggatcaaacggaagcagcaaaggcaaccatgcaggacacttatagggctacactagaacgcttgtttatcgatctagaacaagagcgactactggatcgcggtgccccatgttcttccgagggactatcgtctgtcattgtggatcatccaacgtttcgtcgcatattagacacactgcgtgcgcgtatagaacagacaacaacacaatttatgaaagtgttggttgagacccgcgattataagatccgtgaaggattatccgaagccacccattcaatggcgttaacgtttgatccatactcaggagcattttgtcccattaccaattttttagttaaacgaacacacctagccgtggtacaagacttagcattaagccagtgtcattgtgtattttacggacagcaagttgaggggcggaactttcgtaaccaattccaacctgttttgcggcggcgttttgttgacctgtttaatggggggtttatatcaacacgctctataaccgtaacattatctgaaggtcctgtatccgccccaaatccgacattgggacaagacgcgcccgcggggcgtacctttgatggggatttagcgcgcgtaagcgtggaagttattcgggatatacgagttaaaaatagggtcgttttttcaggtaactgtacaaatctctctgaggcagcccgggcaaggcttgtaggccttgcaagtgcgtaccaacgccaagaaaaaagagtggatatgttacacggggccctagggtttttgcttaaacagtttcacggcctgttatttcctcggggtatgccaccaaacagtaaatcccccaacccgcagtggttttggaccctgttacaacgcaaccagatgccggcagataaacttacacacgaagagattaccactattgcagctgttaaacggtttaccgaggaatatgcagcactaaactttattaatctacccccaacctgcataggagaattagcccagttttatatggcaaatcttattcttaaatactgcgatcattcacagtaccttataaataccttaacttctataattacgggtgccaggcgcccgcgtgacccatcatccgttttgcattggattcgtaaagatgtcacgtccgccgcggacatagaaacccaagcaaaggcgcttcttgaaaaaacggaaaacttaccggaattatggactacggcttttacttcaactcatttagtccgcgcggccatgaatcaacgtcccatggtcgttttaggaataagcattagtaaatatcacggagcggcaggaaacaaccgcgtctttcaggcagggaattggagcggtttaaacgggggtaaaaatgtatgcccgctatttacatttgatcgcactcgccgttttataataacatgtcctagaggaggttttatctgccccgtaacaggtccctcgtcgggaaatcgagaaaccaccctatccgaccaagttcgcggtataattgtcagtggcggggccatggttcaattagccatatacgccacggttgtgcgtgcagtgggcgctcgagcacaacatatggcatttgacgactggttaagtcttacagacgatgagtttttagccagagacttggaggagttacacgaccagattatccaaaccctggaaacgccctggaccgtagaaggcgctctagaagcagtaaagattctagatgaaaaaacgacagcgggagatggggaaacccccacaaacctagcatttaattttgattcttgtgaaccaagccatgacaccacatctaacgtattaaacatttcagggtcaactgtccctggtcttaaacgaccccccgaagatgacgaactctttgatcttagtggtattcccataaaacatgggaacattacaatggaaatgatttaacctccctctttatccaattaaagcccacacgcgggtgagtgtacgtaataaacaagtcaatattacatattctgttgtgttttcttttttgcgtgtagtccttacccatatgacctgtaatatagtgtgtctccaaccattcagcttacagtccagtggacagtaacagcccgataacatggaattggatattaatcgaacattgttggttctactgggtcaagtttatacgtacatctttcaggttgaactgctacgtcgatgtgatccaagggtggcgtgtcgctttttatatcggttagcggctaactgtttgacagttcgttatttattaaagctgtttctccggggatttaatacccagctaaaatttggaaacactcccacggtttgtgcactgcattgggcattatgttatgtaaagggagaaggtgagcgtttgtttgagttgctacaacattttaaaacgcgttttgtttatggtgagactaaagactcaaactgtatcaaagattactttgtctcagcgtttaacttaaaaacctgccaatatcaccatgagctgtcgttaacaacatacggaggttacgtatcgagtgaaattcagtttttacacgacattgagaattttttaaaacagcttaattactgctatattatcacgtcttctcgtgaggcgctaaacacattggaaaccgtgacgcggtttatgacagatactataggaagcggtctaataccacccgtggagttgtttgatccggcgcatccatgtgctatatgttttgaagaattatgtataacagctaaccaaggtgagaccttacatcgtagattattaggatgtatctgcgatcacgttactaagcaagttcgggttaacgtggatgttgacgatattattcggtgtttaccatatatccctgatgtaccggatatcaaacgtcaatccgccgttgaagcgttacgaacacttcaaaccaagacggtagtcaatcccatgggagcaaagaacgatacgtttgaccaaacatacgaaattgcgagcaccatgcttgattcttataatgtttttaaacctgcccctcggtgtatgtacgccatcagcgagcttaaattctggttaacgtctaattccactgaaggaccccaacgtactttagacgtgtttgttgataatttggatgtattaaacgaacatgaaaaacacgcagaacttacagccgtaacggttgagttggcgttatttggaaaaactcccatacactttgatagggcgttttctgaagaactcggatctctggatgcaattgatagtattttggttggcaatcgctcatcctcaccggacagtcagatagaagcattaattaaagcctgttatgcccatcatctatcgtcgcctctcatgcgtcacatttctaacccgagtcatgataacgaagccgccttacgccaacttttagaaagagttgggtgtgaggatgatttaaccaaagaggcgagtgacagcgctacagcatccgaatgtgatctgaacgatgatagtagcataacttttgctgttcatggatgggaaaacctgttatccaaagcaaaaattgacgctgcggaaagaaaacgagtatatcttgaacatctgtctaagcgctctctaaccagcctcggtagatgtatccgcgaacagcgccaagagctagaaaaaacactcagggtaaacgtttatggagaggccttattgcagacatttgtttcgatgcaaaatgggtttggggcacgaaacgtgtttttagctaaggtttcccaggcagggtgtattatcgacaatcgcattcaggaagcggcctttgatgcacatagatttataaggaataccttagttcgacatacagtagatgcggctatgttacctgcacttacacataaattttttgagttggtcaacggcccattgtttaatcacgatgaacaccgttttgcacaaccccctaacaccgccttattttttaccgtggaaaacgttggcctatttccgcacttaaaagaggaattggcaaagtttatgggcggtgtcgttggttccaactggcttctcagtccatttaggggcttttattgcttttctggggtagaaggcgttacttttgcacagagacttgcctggaaatatattagggagcttgtgtttgcaaccacactattcacctctgttttccattgtggggaggtgcggttatgtcgcgttgaccgtctaggtaaggatccacgcgggtgcacgtctcaacctaaaggtataggcagttcccacggacccttagacggcatttatttaacgtacgaagaaacatgtccccttgtggctattattcaaagtggagaaacagggatcgaccagaataccgtcgtaatctacgattcagacgttttttctcttctatacaccctaatgcagcggctggctccggattcaacggacccggcgttttcataacctccgttacgggggtgtggttatgctttttatgcatattttctatgtttgttacggcggttgtgtcggtctctccaagctcgttttatgagagtttacaagtagagcccacacaatcagaagatataacccggtctgctcatctgggcgatggtgatgaaatcagagaagctatacacaagtcccaggacgccgaaacaaaacccacgttttacgtctgcccaccgccaacaggctccacaatcgtacgattagaaccacctcggacatgtccggattatcaccttggtaaaaactttacagagggtattgctgttgtttataaagaaaacattgccgcgtacaagtttaaggcgacggtatattacaaagatgttatcgttagcacggcgtgggccggaagttcttatacgcaaattactaatagatatgctgatagggtaccaattcccgtttcagagatcacggacaccattgataagtttggcaagtgttcttctaaagcaacgtacgtacgaaataaccacaaagttgaagcctttaatgaggataaaaatccacaggatatgcctctaatcgcatcaaaatataattctgtgggatccaaagcatggcatactaccaatgacacgtacatggttgccggaacccccggaacatataggacgggcacgtcggtgaattgcatcattgaggaagttgaagccagatcaatattcccttatgatagttttggactttccacgggagatataatatacatgtccccgttttttggcctacgggatggtgcatacagagaacattccaattatgcaatggatcgttttcaccagtttgagggttatagacaaagggatcttgacactagagcattactggaacctgcagcgcggaactttttagtcacgcctcatttaacggttggttggaactggaagccaaaacgaacggaagtttgttcgcttgtcaagtggcgtgaggttgaagacgtagttcgcgatgagtatgcacacaattttcgctttacaatgaaaacactttctaccacgtttataagtgaaacaaacgagtttaatcttaaccaaatccatctcagtcaatgtgtaaaggaggaagcccgggctattattaaccggatctatacaaccagatacaactcatctcatgttagaaccggggatatccagacctaccttgccagaggggggtttgttgtggtgtttcaacccctgctgagcaattccctcgcccgtctctatctccaagaattggtccgtgaaaacactaatcattcaccacaaaaacacccgactcgaaataccagatcccgacgaagcgtgccagttgagttgcgtgccaatagaacaataacaaccacctcatcggtggaatttgctatgctccagtttacatatgaccacattcaagagcatgttaatgaaatgttggcacgtatctcctcgtcgtggtgccagctacaaaatcgcgaacgcgccctttggagcggactatttccaattaacccaagtgctttagcgagcaccattttggatcaacgtgttaaagctcgtattctcggcgacgttatctccgtttctaattgtccagaactgggatcagatacacgcgttatacttcaaaactctatgagggtatctggtagtactacgcgttgttatagccgtcctttaatttcaatagttagtttaaatgggtccgggacggtggagggccagcttggaacagataacgagttaattatgtccagagatctgttagaaccatgcgtggctaatcacaagcgatattttctatttgggcatcactacgtatattatgaggattatcgttacgtccgtgaaatcgcagtccatgatgtgggaatgattagcacttacgtagatttaaacttaacacttcttaaagatagagagtttatgccgctgcaagtatatacaagagacgagctgcgggatacaggattactagactacagtgaaattcaacgccgaaatcaaatgcattcgctgcgtttttatgacatagacaaggttgtgcaatatgatagcggaacggccattatgcagggcatggctcagtttttccagggacttgggaccgcgggccaggccgttggacatgtggttcttggggccacgggagcgctgctttccaccgtacacggatttaccacgtttttatctaacccatttggggcattggccgtgggattattggttttggcgggactggtagcggccttttttgcgtaccggtacgtgcttaaacttaaaacaagcccgatgaaggcattatatccgctcacaaccaaggggttaaaacagttaccggaaggaatggatccctttgccgagaaacccaacgctactgataccccaatagaagaaattggcgactcacaaaacactgaaccgtcggtaaatagcgggtttgatcccgataaatttcgagaagcccaggaaatgattaaatatatgacgttagtatctgcggctgagcgccaagaatctaaagcccgcaaaaaaaataagactagcgcccttttaacttcacgtcttaccggccttgctttacgaaatcgccgaggatactcccgtgttcgcaccgagaatgtaacgggggtgtaaatagccagggggtttgttttaatttattaataaaaatgtgtattacgttactcatgtgtctccattacgcatcacagggggtatttatacccgataatatacaaaacgcgttttgtacctctaccgcacccgatatcttaacgggggttattatggaatcgtctaacattaacgcgctacaacaaccgtcgtctatcgcacatcatccgtccaaacagtgcgcttcaagtctcaatgaaacagtaaaagattctccccccgcgatttatgaagataggttagaacacacgccggtacaattaccccgcgacggtacaccccgagacgtatgttctgtgggacagctaacctgtcgagcatgtgcaacgaaaccttttcgccttaaccgcgacagccaatacgactacttaaacacatgtccagggggccgtcatatttcactggcactggagattataacgggtcgatgggtttgcatcccgcgtgtgtttccggataccccagaggaaaaatggatggcgccatatattattccagaccgagaacaaccatcatcaggggatgaagattctgacaccgattaaatttaacttaaataaaaccttaccacccataaaaacgccttctgtttgtttaacacgacaccgcttaacaaaaaaaaaacccaaacacgccttttatgaatgtaatacttttatttgttggttaacaccgccccaccatcatctgatttgcaaacatatcggcgtcgtctgccgtggacccctgtattaaaggggccttggaactagcctccactgcatttacatcttgtccaactgtatctgtatgtggggtgcttgttgtattttgggatgagcatagacccgaaacgctttgaagctgttttaataaaatcgatattcgaggatcccgtgtcccctctggtatatttgtatggtgcgacaaaggcatttgtgtcccattttgtgattttagctctgtaacctcctgttgcagttttgccacaaccccagcaagctcttcgtgctgaccattagaaactctgtgtctcctctgccaatatgatggagaaactcgacgtctccgatgcgttatatacgttggttcaccgggaaaatatatatttgagggaaactctccgtccatttgagactccccactagaaaaagaatccaattccctttgatccatgctcttgaaatcccgttttcctggacgacggacatcggttttgtctggaaaatttacacacggggtctgcaagtcaataccccgttcggcggccaatgcgttcataaatgcggacatttgcatttccaaacgattgggtggtggatatcccggaaacccgtacggtcccccgaagtgtcccggagggcaaccataaccccctgtattaaatgggaaggcaggcgggtgtggagatccatatggcccgacgatatactgtccgttatttggagctccaattgatacctgcggatttttagtctgcccggttaacagctgtgaataatacgcggtaggtatcagtacaaattcccctccggttggaacgcccgacgggggctgtggtgagataccactagcgttacctgctacagaagccatatcgctgtcgttcctacacaactgcgtaacctttaaatgcggaacagtcttttcacaatcttcatttgattccccaacacccaacgcgagatcgtatatgggcccgccggggtggaatgtggcgtttataacacccgcgttgggtaatttagactccaccccattaacgttggttatccgagcaagtccatatccggtgctagcctgaagataaacgtgacccataattccggcttcgcgtctacgttttgcaaccacgtcccatctatctcttaaaagcatattgttcacggctgtggataataacaccttggcgagtttatcttcgctaaccttccatactttatttaaacccgcgtagtctttaaccagcgacaataaccgcgctttactttccatcgataaaacccggaatggttcaattgaagattccggggtacagtcataattgaccactgttccaacgcgtcttccaacaacacataacgcaacatgggtaaaaaaattaccgtctggtatctcattcggggacaatcgttttgaagacagggatacggagggtaagtaatttgtgaccaagtataacgcacgttctagcggagataatacagaatctctatttccaaaaaaattcgaatgggccgcttcaaacagcaccgcatgtagttgagggcatctaacgatacccaaaaaaaaaggtccgcgtatgtcctcaatgattgcgattacttcacccacgacacagtcttttcgatgatcgatgtttattggtattttactagtaggcggcaaagcggaccgcacaatctctggggtaatatttaattccccttcgtcctttgaatataaggctaaatacccagccacgtataacgcttcacagttctcttcgtcagcttcagcagccattataaacaccccacggaccggatagtgaatactcacggtgtggaggcaaactgaggaatgacacccaaacagacaaaatatagaagatcatagtcactgttaacgttgaactgcgcaaggcggcgactttcttccaatgccgcccttacacgcggttggtgcattaacattccaagtccccgttcatattgcaacataacactgtcatgtattgataccacggcggctatgggtagggatgtaacattttgtcggcggtgttctaattccaatgcaattaagcttatgagccgatcttggtactgtccagaagaaatatctattacggttcttcctaaacttccacgactaagctgggtatgcgcgtctaaacaaagagcaactaatccaggaaacatttcagtcagctctgtggtccgatttaacgtatacagtggtgctatatatcgttcacataaaaattgaaagttattattaccgcttttaaacttcccatcaaaccccgtcgctccgcgcaagattacattgttggtaggggttcctgttgcttctgacacaatcaaacccagttgaaaattattttttagtttatctccgtatacgttcccgttccataataagcgccttaataataataacgccgtaatcgtgtcaattgttaaccttaatagagtttggtcttccataagaaacacgttttgggcccgttctaaatacgccgcggccgcctgttgaatcttgtccacatatgcggtatgattgcgatcaataatgtcattaaccccaggattaaactgtccaggtgcaggcggtaggacctgcaaccgtataagcgcatccataacagaatgtgacgttaaggcgccttgatcataccgccccccacgagcatgaaactggtcgcgtggtagacgatcatagcaaaattgataactgtttttattttcgtgtgttgtcatataattcacaaatgtctcagtatattccggtaggtgctctataaggttcccgaaggacgaaacttgaggttcgtggacactattagatgtcctatacattaaatataaacataataccgcacactcgaacgcggagtacgctctatctccaacatacattctcccggcggactgtagacatgttaccgttgtgttcataaacgtacgggaaatgcgcccgtctttacaatcaactccgcgtgcagctacgggcctatctaacacaagccgttcctgcagagtacgataccatggcccgaaaacaatccctggagagttattgccccttgcccttcccaagtacaccagggtgataaaatccacttgaaagtttgtatcgtactgcaacggtgcatcatttttggcaatctgtacctcggggtgtatagactcattgcgtattatttctgtacgtgtacattcctcagattgtgcatctgcttcttccgcctcggcagcagccgtctccagggaatccaaaaccttggccatgcgcgttagttgttcttcgaggggctttaaacgacgatctatttccgttggtaacgtaatcgtttccccgcgaaggttgtctaatgcggcaacggccgccgcattttttaacgttaacgtatttttttccaaatcgggattcatacgccctcttaactcaaacgcgggagccgtccagtagtgtatggggaagttgggggctataaagttcttagtggtagacaaaaatatcccacatttattcggaaacgagatagatccgaacccatatctcgccgtcatggtgtctgcagcaaacaaagtcaactggcgtgaatataaaccggtactgctttaaaagctgttttcttacccatgggaaaacatcccggttatactttgtaaaattccaccacaagcacctaaagaaggccttctaaggggtaaatccaccccacaggctgcattttcttcaaactttgttaaagcggaacgatggcatgatttcgcacgctttttcgcaagagagcatacgtgaattttctttttgcatagacgtcttcgctctctaacggaccttatcgggggggtatattccgctacattctccaaatgcgacgctagcataacaaggtttccatgaatcaccttcgggggtaaccgagttacctgtaacaggttcagaccccgttgagatacaaacacaaggaggggggtcaccattatttcatcagatcccgtgggtgtggtttcctttattaaagccacggtatccctcagctggcgcataccctcgcaaaactggtgatacttagtaggggtatgtatattagcgctaaaacggcaagattttaattccactataaaacaaacggtctttccggcaccactggattccgtttgtataatacaaacacaatcggggcgtcggcgtcccaaatttacttcaaacgacattgatatgcgtacagccctttgaacatccacgtgggataacggcgacaggagttttgccagcctcgggttgaacgcgtccgcgaaacctcgacgtacgttatcaatatcctttttgagtacatcgtaaaaacgagtgtggcaacgttgtcccaaacgaaaacacttggcccgaattcgactagcggacatatttgaagttccgtcccagaagataacctaagacgcgtttgtctacaataaacatgtcaacggataaaaccgatgtaaaaatgggcgttttgcgtatttatttggacggggcgtatggaattggaaaaacaaccgccgccgaagaatttttacaccactttgcaataacaccaaaccggatcttactcattggggagcccctgtcgtattggcgtaaccttgcaggggaggacgctatttgcggaatttacggaacacaaactcgccgtcttaatggagacgtttcgcctgaagacgcacaacgcctcacggctcattttcagagcctgttctgttctccgcatgcaattatgcatgcgaaaatctcggcattgatggacacaagtacatcggatctcgtacaagtaaataaggagccgtataaaattatgttatccgaccgacacccaatcgcctcaactatatgttttcccttgtccagatacttagtgggagatatgtccccagcggcgcttcctgggttattgtttacgcttcccgctgaaccccccgggaccaacttggtagtttgtaccgtttcactccccagtcatttatccagagtaagcaaacgggccagaccgggagaaacggttaatctgccgtttgttatggttctgagaaatgtatatataatgcttattaatacaattatatttcttaaaactaacaactggcacgcgggctggaacacactgtcattttgtaatgatgtatttaaacagaaattacaaaaatccgagtgtataaaactacgcgaagtacctgggattgaagacacgttattcgccgtgcttaaacttccggagctttgcggagagtttggaaatattctgccgttatgggcatggggaatggagaccctttcaaactgcttacgaagcatgtctccgttcgtattatcgttagaacagacaccccagcatgcggcacaagaactaaaaactctgctaccccagatgaccccggcaaacatgtcctccggtgcatggaatatattgaaagagcttgttaatgccgttcaggacaacacttcctaaatatacctagtatttacgtatgtaccagtaaaaagatgatacacattgtcatactcgcgtgtacgtgtttttcttttttatatatgcgtcatttattaccacatcctttaatcccgcctttatctccctaaaacggagtggtaatattaaaagccgccaagcctgttggtgggtgaggaggggtaaaggcacgctgtgtgcataacgttgcggtgatattgtagcgcaagtaacagcgactatgtttgcgctagttttagcggtggtaattcttcctctttggaccacggctaataaatcttacgtaacaccaacccctgcgactcgctctatcggacatatgtctgctcttctacgagaatattccgaccgtaatatgtctctgaaattagaagccttttatcctactggtttcgatgaagaactcattaaatcacttcactggggaaatgatagaaaacacgttttcttggttattgttaaggttaaccctacaacacacgaaggagacgtcgggctggttatatttccaaaatacttgttatcgccataccatttcaaagcagaacatcgagcaccgtttcctgctggacgttttggatttcttagtcaccctgtgacacccgacgtgagcttctttgacagttcgtttgcgccgtatttaactacgcaacatcttgttgcgtttactacgttcccaccaaacccccttgtatggcatttggaaagagctgagaccgcagcaactgcagaaaggccgtttggggtaagtcttttacccgctcgcccaacagtccccaagaatactattcttgaacataaagcgcattttgctacatgggatgcccttgcccgacatacttttttttctgccgaagcaattatcaccaactcaacgttgagaatacacgttcccctttttgggtcggtatggccaattcgatactgggccaccggttcggtgcttctcacaagcgactcgggtcgtgtggaagtaaatattggtgtaggatttatgagctcgctcatttctttatcctctggactaccgatagaattaattgttgtaccacatacagtaaaactgaacgcggttacaagcgacaccacatggttccagctaaatccaccgggtccggatccggggccatcttatcgagtttatttacttggacgtgggttggatatgaatttttcaaagcatgctacggtcgatatatgcgcatatcccgaagagagtttggattaccgctatcatttatccatggcccacacggaggctctgcggatgacaacgaaggcggatcaacatgacataaacgaggaaagctattaccatatcgccgcaagaatagccacatcaatttttgcgttgtcggaaatgggccgtaccacagaatattttctgttagatgagatcgtagatgttcagtatcaattaaaattccttaattacattttaatgcggataggagcaggagctcatcccaacactatatccggaacctcggatctgatctttgccgatccatcgcagcttcatgacgaactttcacttctttttggtcaggtaaaacccgcaaatgtcgattattttatttcatatgatgaagcccgtgatcaactaaagaccgcatacgcgctttcccgtggtcaagaccatgtgaatgcactttctctcgccaggcgtgttataatgagcatatacaaggggctgcttgtgaagcaaaatttaaatgctacagagaggcaggctttattttttgcctcaatgattttattaaatttccgcgaaggactagaaaattcatctcgggtattagacggtcgcacaactttgcttttaatgacatccatgtgtacggcagctcacgccacgcaagcagcacttaacatacaagaaggcctggcatacttaaatccttcaaaacacatgtttacaataccaaacgtatacagtccttgtatgggttcccttcgtacagacctcacggaagagattcatgttatgaatctcctgtcggcaataccaacacgcccaggacttaacgaggtattgcatacccaactagacgaatctgaaatattcgacgcggcatttaaaaccatgatgatttttaccacatggactgccaaagatttgcatatactccacacccatgtaccagaagtatttacgtgtcaagatgcagccgcgcgtaacggagaatatgtgctcattcttccagctgtccagggacacagttatgtgattacacgaaacaaacctcaaaggggtttggtatattccctggcagatgtggatgtatataaccccatatccgttgtttatttaagcaaggatacttgcgtgtctgaacatggtgtcatagagacggtcgcactgccccatccggacaatttaaaagaatgtttgtattgcggaagtgtttttcttaggtatctaaccacgggggcgattatggatataattattattgacagcaaagatacagaacgacaactagccgctatgggaaactccacaattccacccttcaatccagacatgcacggggatgactctaaggctgtgttgttgtttccaaacggaactgtggtaacgcttctaggattcgaacgacgacaagccatacgaatgtcgggacaataccttggggcctctttaggaggggcgtttctggcggtagtggggtttggtattatcggatggatgttatgtggaaattcccgccttcgagaatataataaaatacctctgacataaaaaacatgtataataaaaagtcactataaacgtattctctacaatactttattcgcgaataatacacactacctttgggtttttttcccgtccccaaatggtgtttggtgcactctaccaaaaaatagagcgcctaaatatgctatataacgcctcccagcaaaatacggttcaaaggcattacccgatattgtattgtagtacagggcaatgggaattgatgatcccaataaacggcatagacgcacagcgccgttatagcaggggtctccagagtacagggtatctaagtaccgggatatctcatactcatgcctttccgtgacagaaacatcaaccggaacagtatccgataaaccaactcctgtttttgcaaggcgtaaaattcgcacaccttccttttttgcaagatgtgacgtttccttgtaacagggaagctgggggagtggtaagaacaacaaagtttcagccaacgtgccaataaagcccacttccctcaagaggctgtttgctgtatccacaatggtccgtattaaatcttgagcaacttgatccgtgtcatcatcactgggtaacgcgttaacataactacgcgttaaatcttcaataacggcataacaattaaacgcttcccaccgagacagtatatattgaacaatcacgaaccgttgacaggacgtcagatcacgtccgtaagcatgcccgaaaaatggaagttccccccgttcgccatataccgcaacaactgcggtatatatcgtctcacgggcttcattaagttcatcttcaagtccaggccattttctggctttaaatataacctcgtccgcaaaaaaaaccgcacatgataacgcgcggatacaatgagtagtggctttatggcgaggatcccaaatgtccattacccgggggatggtcctaatctgtacaaagttacttagtgtaatatgatcggacttcttacgccgtctaggctgtttctcagaatacggttcacccgaaatcggcacatcatctgcttttacgtcttccgtaaccacatcagcagcgcgccgactaacaattatacttgttttttcatcgtcgttacttccgttaagcgcgtctcgtatctcgggcgtcccgtcgaataatccactcactagctcctgcaaaccttctggtaactccaacatacgcatatacaccaatgaaaaactggcttcgtttggtacgtacataaagccatttgtggtattaatggcggtgggtgttggaaacaattttagcttattctcgcgcgtaacatctacccccgccaccaatgttaaatgcgtcacggggagggacacgagataatctgcgagcgtagggtcctccacttcaacatcaaatgttccgcaaaggtcgcgatccaccgcccccgatcccgctgcaagtaaggccactcgatccaaaaacacgcagttattattggatgataccgcccatgtcttcccggtgcgattgagctcacttcgaacgtaactggcaacagatctgtcaccgggtccgaccccgcgaacaacatgtccaaattttgcgatctcgcctccatgtttgcggggtatggaaattaagcatcccccgcatataaaatacgccctggtagcacgctcgttaaaataaaacgttacgccgttataagatacggttgaatgatatggaaattccatattaaagcgtttatcggaacattaacctcgaacttgccgtcccgtgatcgtgtgatcgccaaccttaggtccacaccgaatatgagaaatatataactacacgcaaacattcaaaacaccgtggtatcattaacgtcatatgaaaagatccaatcaatccaatcaaccacacctcctaccgtttagcacgtcagctatgtgacatgctccaaacatacgtaaacatttagagagggtgttataacagtctgtcaggcggggtatattctacataatacaaggatcggctttaactttgtcaacatttttactttggactataaactgcgactgaacgttatgaacccaccccaagcccgcgtctcggaacagacaaaggacttgcttagcgttatggttaaccagcaccccgaagaggacgcaaaagtgtgtaaatccagtgataattcaccgctttataacaccatggttatgttatcgtatgggggtgatacggacttactattaagctctgcatgtacccgcacatctaccgtaaacaggtcggcgtttacgcaacactccgtgttttatattatatccacggtgttgattcaaccaatatgttgtatcttcttttttttttactataaagcgacacgctgtatgctcttattcacagccgggttacttctgacgattctacatcactttcgacttattattatgttattgtgtgtctacagaaatatacgatcagacctgctacccttatctacatcccagcaactgctgcttggaattattgttgtgactcgaacaatgctattttgtattacggcgtattatactctttttatagacacccgggtgttctttttgattaccggacacttgcaaagtgaggttatttttccagatagcgtttcaaaaatacttcctgtgtcgtggggtccaagtccagccgtgttactggtaacggcggcagttatttacgctatggactgtttggtggacacggtatcctttattgggccaagggtgtgggtccgtgttatgttaaaaacatctatttcgttttagtccatttcaataaatgtactataattgttcagtctaaaaataatgttgggtatttataattaccgcccccgtgttacttggaaacacccatacatatgttccactctacatcaaacttctcgcagttttcttgttcccgcacacgtttacacgtccggattcaagtcgcaacgctgctgacaaaatgacaacggtttcatgtcccgctaacgtgattactacaacggaatctgatcgtattgctgggttatttaacatcccagcggggatcattccaactggaaatgtgctgtcaaccatagaggtgtgtgcacaccgttgcatttttgatttttttaaacaaatacgatcagatgataacagcctttactcggctcaattcgatattcttttggggacatactgcaatacattaaactttgtgcgttttctagaacttggactgtctgtcgcttgcatctgtactaaatttccggagctggcttacgtgcgagatggcgttattcaatttgaggtacaacaacccatgatagcacgtgatggcccacatcccgtcgatcagcctgttcataattatatggttaagcggatacacaagcgttcgttaagcgctgcgtttgcaattgcatcggaagcgttgagtttgttaagtaacacatatgtcgatgggacagagattgactcatcgttacgtataagagctatccaacagatggctcgtaatttacgcaccgttttggactcatttgaacgaggcactgccgatcaacttcttggtgttctattggagaaagccccaccgctatcgctgctttcaccaattaataaattccaacccgagggacatctaaatcgtgttgcacgcgcggccctactttcggacctcaaacgtagagtctgtgcggatatgttttttatgacccgacacgccagggaacctaggctgatctctgcgtatctgtcggatatggtttcgtgcacccaaccatcggtgatggtatcacgaataactcatacaaacactcgcggacggcaggttgacggtgtgttggtaacaacagcaaccttaaaacggcaactattacagggaattttacaaattgacgacaccgccgctgacgtaccagtaacatatggcgaaatggttctacaggggacaaacttggtaaccgcccttgtgatgggaaaggccgtccgcggaatggatgatgtagcccgccatctccttgatataaccgaccctaacacgttaaacataccgtctatacccccacaatccaactccgattcaacgacagctgggcttccggttaacgcccgtgttcctgcggatttagtgattgttggggataaacttgtattcttagaagcattagaacggcgggtctaccaagctacgcgcgttgcctaccctcttattggaaatatagatattacgtttatcatgccaatgggagtgtttcaggcaaactccatggacagatatacacgacacgccggcgatttttcaactgtatccgaacaggatccacgtcaatttccaccccaagggatttttttttataataaagatgggatattaacacagttgactcttcgtgatgcaatgggtaccatctgccacagttcattgcttgatgttgaggccacacttgttgccctccgccaacaacatttagatcgtcagtgttattttggtgtatacgtggccgagggtacagaggacacattggatgttcaaatggggaggtttatggaaacgtgggcagatatgatgcctcatcaccctcattgggtaaacgaacatttaacaattctacagtttatagctccgagcaacccgcgtctaaggtttgaattaaaccccgcctttgatttttttgttgcaccgggggacgtagaccttcccggaccgcagcgtcccccggaagccatgccaaccgttaacgcaacattacggattatcaacggaaacattcccgtgcctctatgtcccatttcatttcgagactgtcgcggaacccaactcggtttgggaagacatacaatgaccccggcaaccattaaagccgtaaaggatacatttgaagaccgcgcatacccaactattttctacatgctagaggctgttattcatggaaacgaaagaaacttctgtgcgttactgcgactgttaacacagtgtattcgcgggtattgggagcaatcccacagggtggcatttgtaaataactttcacatgttaatgtacataactacatatctcggaaacggtgagcttcccgaagtctgtattaatatatatcgggatttactgcagcatgtaagagcattacgccaaactataaccgattttacaatacaaggagagggccataacggcgagacctcggaagcgctaaataacatccttacggatgacacgtttattgcacctattctatgggattgtgatgcgttaatataccgtgatgaagccgcccgagaccgactccccgcaattcgtgtaagcgggcgaaacggataccaagcccttcactttgtggatatggccgggcataacttccaacgacgcgataatgtgttaatccacgggagacccgttcggggagacacgggtcagggtattcccattaccccacaccatgaccgtgaatggggtattctctccaagatttactactatattgtcattcctgcattttcccgcggttcctgttgtacaatgggcgtgcgttatgatcgcctataccctgcgttacaggcagttatcgttccggaaattcccgctgatgaagaagccccaactaccccagaagatccaagacaccctcttcacgcacaccaactcgttccgaactctcttaacgtttacttccataatgcacacctaaccgttgatggtgatgcattgctcacactacaagagttaatgggagatatggctgaacgaacgacggccattttagtatcaagcgcccccgatgcgggagccgccacggcaacaaccagaaatatgagaatatatgacggagcgctttaccatggccttattatgatggcatatcaggcgtacgatgaaaccattgcaacgggtacttttttttatcccgttccggtcaaccctctgtttgcatgtccggaacatttggcatcattgcgtggaatgacaaatgctaggcgggttttggcaaaaatggtaccaccaatccctccttttctgggagccaaccaccacgcaactatacgccaacccgttgcctaccatgtaacgcatagtaagtcggattttaatactcttacatattctcttcttggagggtattttaagtttacaccaatatctcttacacatcaactacgaacgggatttcaccccgggattgcctttaccgtagtgcgccaggatcgctttgccacagagcaacttttatatgccgagcgtgcttctgaatcgtactttgtcggacaaatccaagtacaccatcatgatgctattgggggggtaaactttaccctaacccaacccagagctcacgtggacctgggagtcgggtatacagctgtatgtgccacagcagccctgcgatgccctctcacggatatgggcaatactgcccaaaatctttttttttcacgaggaggagtgccaatgttacatgataacgttaccgaatcgttgcgtcgtataacagcatcggggggtcgcttaaatcccaccgaacccctacccatcttcggcggactacgtcctgctacatcggcaggaattgcacgagggcaagcctctgtgtgtgagtttgtggccatgccggtgtccactgacctacaatattttagaactgcatgcaatcctagaggtcgagcatctggaatgttatatatgggtgaccgtgacgccgacatagaggctataatgtttgatcacacacaatcggatgttgcttatacagatcgagcaactcttaacccatgggcatcacaaaaacattcatacggtgacaggctatacaacggaacatacaaccttacaggcgcttctcctatctacagcccatgctttaagttttttacaccagcggaggttaacactaattgtaatacactggatcggcttctaatggaggcaaaggctgtggcgtcgcaaagctccaccgacactgaatatcaatttaaacgccctcccggttctaccgaaatgacacaggatccgtgtggcctttttcaagaagcatatccaccactatgctcaagcgatgcggccatgttacgaacggctcacgcgggagaaaccggggcagatgaagttcacttagcccaatatctgattcgagacgcgtcgccccttaggggatgtcttcctcttccgcgataatttcaccacgcccacatacccactcccaataaaagccctgtagagcgcattggcatcttacttgagatttggatacgctcggccgacttggtctgtttcacgcttccttaaacaacatggctatgccatttgagatagaggtattgttaccaggagaactatccccggcggaaacatctgcattacagaaatgtgagggaaaaattattaccttctcaaccctgcgtcatcgagcttcactggtggatatagcgctgtcgtcatattacattaacggtgctccaccagacacgctctcgctgttagaggcataccgaatgcgattcgcggcagttataacacgggtcatcccgggaaagttgttggcgcatgccattggcgtgggtactcctacacccgggttgtttattcaaaatacatcccccgttgatctttgtaatggcgattacatctgcttacttcctccggttttcgggtccgcagactcaattcgcttggactctgtaggactggaaattgttttccctttaaccatcccccagaccttaatgcgagaaatcatcgccaaagtggttgcacgggccgttgagcgcacggccgcgggtgctcaaattttaccccacgaagttctacgaggcgcggatgtcatttgttacaatggaaggcgttatgaactcgaaacaaatttacaacatcgggacggatcggatgcggctattcgcacattggttttaaatctaatgttttccataaacgagggatgtctgcttttattggcgctgattccaactttgttagttcaaggagcacacgacggttatgtaaatttattgatacaaacggccaattgcgttagagaaaccggccagttaattaatataccgccaatgccgcggattcaagacggccatcgccgatttcccatatatgaaactatttcatcttggatatcaacatcatctagactgggggataccttgggaactcgcgcaattttacgcgtctgtgtgtttgatggaccctctactgttcatccgggagaccgcacggccgtgattcaagtgtaaacaggtgttaataaaaacacaaccagtctagttacatttcacgcgtcttgtttttatttaataggcataaacacggaatccggtatacatgaactgccaatatacacggacataattaatgcaaccatcagatcatctgacattgttcccgtggtacctttacccgtgtaagtttttgtgtctagattacccataccgcctttaattacctctgtcaggttatccaactgtttacatagatactccacggggtctacacctaactttactgttagggatacaagctcctgtgaggctattatatttccggagttaaatcgtttaacaaaatagtctacggccggcgttttttgtttttgtaataaaaaaaaagggtacgccacgctacatccgggaggtatggaatgataaaacagtaacactggagcggaagatagcacgtttcccttttcgaggacagcaaactgttgtgctatagccaacgatatggcaactgcagaatcctggctgctgtttccctctatagaaacgtgtacgtttgtaaatgtattggggtgtaaagcgagtatgtggcctaagcattgagtaacgcaacgccctatctcactggaagacgtgccagttaaagctctaagaaaaaagtgctccaatccaaatataatccaatccgacttataacgaccaacaatcgctacaccagtaccagacgctcgtgtatttgaggtaaatgcagggtctacgtaaacgtacaacactgacgataatatagcacaattcgcaacggttgacggccgatataaaataaacctctcacgggcagtttttgtaaataatggccggtcaaaccccacacccccagaattctgtttacgcccacctacaatttcctgcacgaaggagtcggccataaataaatctgcagtgcgccgcatggctccatccattgtgatgaaaaccggcttatttaatacataacacgaacaagctgtgacatcgctatgtgctaaaacacgcggcatgtgatcgtcgcatacatatgtaacaacgtttaacaactgatccgacgatccacgtaagttatacaaaaaacttgtacttgcttttccggtatttgttgatgaaacaaaaataattttacaattggtttgatttaaaaatccgactatagtttgtacagcatcaggtcgaataaaattagcttcatccacaaacagaagattaaaatcttgacctcggataccctggaacgatagaaagatatatagttaccccaccaaagtttaaatgtatccttaaataccacgtacgtaaaaaatgtttgaatacgtacatatttctttttttttccagtacaaccatatccggtgtataatggaagcccatttggcaaatgaaaccaaacatgcactttggcataatgatcacacaaaaggattactacacgttgtgatacctaacgcggggcttattgcggccggaatagatcccgcattactgattttaaagaaacccggacaacgcttcaaggttgaagtacaaacaagatatcatgctacaggtcaatgcgaaccgtggtgtcaagttttcgccgcgtacattcccgataacgccttaacaaatctcttaataccaaaaacggaaccatttgtttcacacgttttttcggccacgcataattcagggggattgattttatcattgcctgtttatcttagccccggtttattctttgatgcatttaacgttgtagcgatacgaataaatactggaaaccgcaagcaccgtgatatttgtattatgtatgcagaactaatcccaaacggaacgcgttattttgctgatggacaacgggtacttttattatgcaaacagctgattgcgtatatccgatgcacccctcgtcttgcatcgtctataaaaatatacgcagagcatatggtggcagccatgggtgaatcacacacgtcaaatggggacaatattggacccgtttcatccataatcgatcttgatcgacagttaacttctggaggtattgatgactcccctgctgaaacacgcatacaggaaaataatcgggacgtccttgagctaataaaacgggccgtaaacattgttaactccaggcaccccgtccgaccttctagttcccgcgttgcatctgggttgcttcaaagtgcaaagggccacggagcgcaaacttccaacacagatccgatcaataacggttcctttgatggcgtccttgagccgcctggacaagggcgatttacgggaaagaaaaacaattcgtccgccagcatcccacctttacaagacgttctattgtttaccccagcttcgacagaaccccaaagtcttatggaatggttcgacatctgttatgcccaattagttagcggggacactccagcagatttctggaaacggcgtcccctatcaattgtaccgcgacattacgcagaatcccccagtccgttgattgtagtatcttacaacggatcctctgcctggggaggacgtattaccggaagtccaattttatatcactctgcacaggctattattgatgctgcgtgtataaatgcccgggttgacaatccccaaagcctacatgtgacagctcgccaagagctagtcgcgcgtttaccgtttttggctaacgtcctaaataatcaaacccccttacccgcctttaaaccaggcgccgaaatgtttttaaaccaggtatttaaacaagcgtgtgtgacatcgctaacccaaggtcttataacggagttacaaacgaacccgactctacaacaactcatggaatatgatattgcagattcttcccaaacggttattgatgaaattgtagcccgcacaccagacctgattcagactatagtttcggtgttaacggaaatgtcaatggatgcgttttataacagctccttgatgtatgcggttttggcgtatctgtcatctgtatatacacgaccacaaggtggggggtatataccctaccttcacgcttccttcccatgctggttaggtaatcgttctatatatttatttgactattataattcaggaggggaaatacttaagctttccaaggtccccgttcccgtagccttagaaaaggttggtattggtaattccacacaactgaggggtaaatttatacgcagcgcggatattgttgatattggaatttgttctaagtatttacccggtcaatgttacgcgtacatttgtctaggatttaaccagcaattacaatccattttagttttaccggggggatttgcggcatgtttttgtattaccgataccctacaggcagcactacctgcatcgttaatcggacctattctagacagattctgcttctctattcccaacccccataaataaattagtgtcactataaaaacataacaccagaatctcttcatatttaattttacgtcatttctcccgtttccaccccctcttaaaatataaaataaccgggtgggtggcattaaacccacaagtacccgggcggcaatccgctagactgtttttctgctcatggaattacaacgcatatttccgctgtacaccgctacgggtgcagcgcgcaaattaacccccgaggcagttcagagactctgcgatgcattaacgctggatatgggattatggaagtccatcctgaccgatccccgggtgaaaataatgcgatcaactgcttttataactttaaggatcgctccgtttatcccccttcaaacggatactactaatattgccgttgttgtagccacaatttacatcacgcgcccacgtcagatgaacttacctccgaagacttttcatgtaattgtaaattttaattacgaggtctcgtacgcaatgacggcgactttaagaatttatccggttgaaaacatagaccatgtttttggagcaacgtttaagaacccgatcgcgtacccccttccaacatctattccggatcctcgagcagatcccacccccgcagatcttacaccaacgccaaacttaagcgactacttacaacccccgcggcttccgaaaaatccatacgcatgtaaagttatttctccgggagtgtggtggtcagacgaacgaaggcgtttatatgtactggctatggaacctaatttaatagggctatgtcccgccggatggcatgctcggatacttggctctgtattaaatcgactcctcagccatgcggacggatgtgatgaatgtaatcatagagttcacgtgggggcactgtatgcgttaccccatgtcacaaatcatgcggaaggttgtgtgtgttgggctccgtgtatgtggagaaaggccggtcagcgggaattaaaagtggaggtagacattggcgctacgcaggttctttttgtagatgtcaccacctgcattcgaattacgagtactaaaaatcctcgcattaccgcaaatcttggcgacgttatagcgggaaccaacgccagtggtctctctgtaccagtaaattcatctgggtggcagctttatatgtttggagaaacattaagccgggctattattaacggctgtggtctgcttcagcgaatttgcttccccgagacacaaagattatcgggtgaaccggaacctacaaccacctagtataccttaactcaaccgccgttgtggaaaggtatatgtcaacatttacagtaatatattaaaggttaaatttataaaacactcacgtttgtgttgtgacttgacgcgaacaccgctgtgctgtaagacccgtcggtaaatgaaaacgtaatagattcgccttttacatgatccacgtaatttgccccaaaccactgttccaggcgagacttgataccctcaaacacgggttccgttgctttgcgtatatgagccgtataacccactttaattcctctaaacgtggccattactaaagctattaatggtacaagaaaccatgttttcccatgtctacgtggtaccaaaaacacagttgatttttgtttgaagtgttctaaaacactgtcagaaacacttggcgtgttaaacactgtacgcagaaagcagtcaactctgtcggcatgatcgcccaatagcaccgatgaaataaaatgcgtggtgtgcatgaggatcattttttgaaacagttccaacgtccccttatatctgccatagattggaacgtcaacctttgcgcgtttgccatgacttccacactcttcaatactctcaaaagatgtttccacaaggtacgaaaaccgttgtgtaaaggtagacaactgacagaaactatccgacagagaaaacgcgcgaaatgtgttcataacaccgctatacgcatttcgatgaggtgctgcttcttccggtgaatattcataaaactgtacactactgacagccttttttaattcggggcttacgtttgcatttaccgaatatcgccatggtttcaaaactacattgggggtacagttgtaccctgttgacgatagaaacgcgccaaacattgcccgtcgagcagtagccgagaacagtggaatatattcacaacagttgtgaagcgttccaattccgggaataacggcctgatgacgtcgggttacatctatagcaaaattcagaaacgggatttgggttgcgtttcccagagacccttgccgcgtggaacacggggtaggggactccaacgtcccaaagcgttcatccctacgacgctttagacgttcaaaatatcttacagattcttcaccaagcgtacgaccaaacattatcaatgacatttaacatcaattcacggaatccgcctcatctcttgtaagcagtaaaacaggaagccgcgtcatcttacgtactcgttacgtatatatcataaacattttcagggccgcattcattcactttggtcatgtcaggccacactccaacctacgcttctcataggcgtaaccgtgtcaaactagttgaggcgcataaccgcgcggggttatttaaagaacggaccctcgatctaatccgtgggggtgcgagtgtacaagatccagcatttgtgtatgcctttactgctgcaaaagaggcctgcgccgatttaaataaccagctccgctctgcagctcgcatagcttcagttgaacagaagattcgtgatatacaatccaaggttgaggaacaaacaagtattcaacagattttaaatacaaacagacgctatatagcacccgattttattcgcggtttggataaaacagaagacgataataccgataatatagacagactggaagacgcggtaggaccgaacatcgaacacgaaaatcatacttggtttggagaagacgacgaagcgttacttacacaatggatgctgacgacacaccccccaacctccaaatatctccaactgcaggacctttgcgttcccaccacaataccgacggacatgaaccaaatgcaaccgcagccgatcagcaagaacgagaatccaccaaccccacacacggatgtgtaaatcatccatgggccaatccgtcaactgcaacatgcatggaatcaccagaacgatcacaacagacaagcttatttttattaaagcacggcttaacgagagatccaatacatcaacgcgaaagggtggacgtttttccacaatttaacaaacccccatgggtttttagaatttccaaattatcccgtttaattgtacccatcttcacgctcaatgaacagttatgtttttctaaattacagattcgagatagacccaggtttgcgggacggggaacgtatgggcgtgttcatatatacccatcgtcaaaaatagctgtaaaaaccatggacagtcgtgtttttaatagagagttaattaacgcgattttagcgagtgagggttctatacgagcaggggaaaggctaggtatttctagcatagtttgccttttaggtttttcgttacaaaccaaacagctactgtttccggcatacgacatggatatggatgaatacattgttcgcctgtccagacggttgacaatacctgatcacatagacagaaaaattgcccatgtatttttagatttggctcaagcgttgacgtttttaaatcgaacgtgcggcctgacccacctagatgtgaaatgtggcaatatttttcttaacgtcgacaactttgcctcgttggaaataaccacagcagtaatcggagactatagcctagtaacattaaatacgtattccctttgtactcgagcgatatttgaagttggaaatccatcccacccggagcacgtactacgcgtaccccgggatgcatcgcagatgtcatttcgtttggtgttgagtcatggaacaaaccaaccccctgaaatcttgcttgattatattaatggaacgggccttactaaatatactggaaccttgccccaaagagttggacttgcgattgatctttatgcattgggccaagcactcttagaagttatcctgctaggacgtcttcccggacaactgcccatttcagtacatcggaccccgcattatcactactacggtcataagttatcaccagatttggcgcttgatacgctggcatatcgatgtgtcctggcgccatatatactcccatctgacatccccggggacttaaattataatccctttatacacgccggagagctgaacacccgtatttcccggaattctttacgccggatattccagtgtcacgcagtgcgttacggcgtaacgcactcaaagcttttcgaaggcatacgcattccggcctcattatacccagccactgttgttacgtcgttgttgtgtcacgataattcagaaatacgctcggatcaccctttattatggcacgatcgggattggataggatcgacataagcccccagccagccaaaaaaattgcccgtgtgggaggtctacagcacccttttgtaaaaacggatattaacacgattaacgttgaacaccattttatagacacgctacagaagacatcaccgaacatggactgtcgcgggatgacagcgggtatttttattcgtttatcccacatgtataaaattctaacaactctggagtctccaaatgatgtaacctacacaacacccggttctaccaacgcactgttctttaagacgtccacacagcctcaggagccgcatccggaagagttagcatccaaattaacccaagacgacattaaacgtattctattaacaatagaatcggagactcgtggtcagggcgacaatgccatttggacactactcagacgaaatttaatcaccgcatcaactcttaaatggagtgtatctggacccgtcattccacctcagtggttttaccaccataacactacagacacatacggtgatgcggcggcaatggcgtttggaaaaaccaacgaaccggcggcacgagcgatagttgaagcattgtttatagatccggctgatatccgtactcctgatcatttaacgccagaagctacaactaagttttttaattttgacatgctcaataccaaatctccaagtctccttgtgggtacaccaagaatcggaacgtatgaatgtggacttttaatcgacgttcgaacgggacttataggcgcgtcgttggacgttcttgtatgtgacagggaccctttaactggcaccctaaatccccaccctgcagaaaccgacatttcattttttgaaattaaatgtcgtgctaaatacctctttgatccagatgataaaaataacccgctcggtcggacgtacaccacgttaataaatagacctacaatggcaaatctacgggactttttatatactataaaaaacccatgtgtaagcttctttggaccctcagcaaacccaagtacacgcgaggccttaataacggatcacgttgaatggaaacgtttaggatttaaaggtgggagggcccttacagaactcgacgcccatcatttgggcctcaatcggacaatctcatcccgagtgtgggtatttaatgatccggacatacaaaaggggacaattacaaccattgcatgggccactggagatacggctcttcaaattcctgtatttgccaatccgcggcacgctaactttaaacaaattgccgtacaaacctatgtattatccggttactttccagcgctaaaactacggcccttccttgtcacctttataggacgtgtgcgccgaccacacgaggtgggagtcccattgcgcgtcgatacacaagcggctgccatttacgaatataactggccgactatcccaccccactgtgcggttccggttatagccgttctaacgcctatcgaagttgatgtgcctagagtgacaaaaatacttaaagacacaggaaacaacgcgattacatcagcattgcggtcattgcgatgggacaatcttcatccagcggtcgaggaggaatctgtggattgtgcaaacggtacaacgagcttgttacgtgcaacggagaaaccgttgctttgaactcagagttctttgaagactttgactttgatgagaatgtaacagaggacgccgataaatccacacaacgccgcccacgagtgatcgatgtaacaccaaaacgaaaaccttcgggaaagagctcccattccaaatgcgcaaaatgttaaaccctgataaacgttctaataaaaacatcaaatcatggttggttactgtgaatgtttgttttattgcttggggggttacaagtacaacccacgctactcccacccactgtttgatcgctcgtataacagctcatcctcgcggtccgtttcatatgttgagtcattttcatagacgtagccgtagccttgtgatgggtaatttgtgcggcgagaatttctatgtgcaggttttacttttcgtatgtatccccgtacccgctcgggtactcttcttacggcaccgtagaaccgactgcgtttctgtcgatgatacacatatgcacgcatcaatctgagaagcaacatgacaacggaaaacacggccaggcaagccaaggttccccgagttgtgggaattaaccgtggagattgaaccgatatagggtcatataatcggtccatatacgagtgcgcggcggttcccaacgtagcacaggccacgagcgttcccagggacggtcctattaacacgtgtatataatgcgccaaaattaattctgatactataagatatacaactgacaatgtactaaatgtagacatggccacggacaccgatgaccacagtcccgtatgtagatgattcgccaccacaagttccagcattaatgatacaaataggatacatatcgccatcaacgcagccatcaaattcacgaacactgcgcgcgtaggccccgcaaggcgatataaaaagacgctctgctgtcgtaaatttgcgaccgcttttatgttcgtttcgtccaattttccgcgtccacaaaaatacgttgtaaatattacacttgtcgcaaaatgtccaagatataatgtagcagccacgccgatttgcttgtaagctaataataacacaacggcgtttaataaccacaatgacaaaagaccccaaaaaagtgttgtgggatctacaactaaccatgcaacaccggagctttgccggacacgttgatttttcgtttctcggtgtataatcgcggccgtgatcagtgtatataccgccatggccattgccgttaaagccgtgtagtaagtaaatgccacaacgctatgtggttccaaaaacaaaaccggggcgctgtatccacctctatttccggaccatacccccccatctagggtggcgttaaataactcataatcaactacggcagcataaaaacaagggatcccggtatattcagaagaggcggcaattaacgtagccaggagcattaccgcacccaaagtgaacatcatcacctgaattatccaaattcgccaattaagcgtatccatttgatgatctaacgcttccacctcgggtgtcgtggtgtcgtacggtgagactttttcagaacgcggccccttcttttgagttcccatgtctcccaacaccggggagagcaacgccgccgtctatgcgtccagtacacagctcgcgcgggcgttatatggaggggatctggtttcgtggattaaacacacccacccgggaattagcctggaactgcaattggatgttccagtaaaactaataaaacctggtatgtcacaaactcgcccggtaaccgtcgtacgtgcccctatgggctctggtaaaacaacagccttgcttgagtggcttcaacacgcgttaaaggcagatattagcgtactggttgtctcatgtcgccgtagctttacccagacgttgattcaacggtttaacgatgcaggcctctccggattcgtaacatatttgacatccgagacatatattatgggttttaaacgtttgattgtgcaacttgaaagcctacaccgcgtatccagcgaagctatcgacagctacgacgtattaatactggatgaggtaatgtcagtgattggacaattatactcccccacaatgagacgtctttccgcggttgatagcctattatatcgtcttttaaatcgctgttctcaaattatcgcgatggatgctacagtaaactctcagtttattgatttaatctccggattgcgtggagatgaaaacatacacacaattgtgtgtacatacgcgggagttgggttctccggaagaacttgcacgatcctgcgtgatatgggcatcgacacgcttgtgcgagtcattaaacgatctcctgaacacgaggatgtacgtaccatacaccaactacgtggaacattttttgacgaactagcactacgattacaatgtgggcataacatctgtatattttcatcaactttatcgttttcggagctagttgctcagttttgtgcaatatttacagactctattcttattttaaactcaactcggcccctatgtaatgtaaacgaatggaaacattttcgcgtgttggtgtacactaccgtcgtgaccgttggattgagttttgacatggctcattttcatagcatgtttgcttacataaagccaatgtcatatgggccggatatggtatcggtctaccagtcattagggcgtgtacgtttattgctacttaatgaagttttgatgtacgtcgatggctcaaggaccagatgcggacccctgttctcgccaatgttactaaactttaccatcgcaaataaatttcaatggtttcctacacacacccaaataactaacaaactgtgctgtgcatttaggcaacgatgtgcaaatgcatttacacgctcgaacacccatctcttctcaagatttaaatacaaacaccttttcgagagatgctctctttggagtttagccgatagcattaatatcttacaaactcttttggcctctaaccaaattttggttgtattggatggcatgggtccaataacggacgtttccccagttcaattttgtgcatttatacacgatctcagacatagcgctaacgccgtagcttcctgtatgcgttctcttagacaggacaatgacagctgcttgaccgattttggcccttccggatttatggccgataacattaccgcgtttatggaaaagtatcttatggagtcaattaataccgaagaacaaattaaagtatttaaagcccttgcatgtccaatagaacagcctagactagtcaatacggcaatattgggggcgtgtatacgaatacctgaagcgttggaagcatttgacgtatttcaaaaaatatacacgcactacgcttccggttggtttcccgtcctggacaaaaccggggaatttagcatcgcgactataactaccgccccaaatttaaccacgcattgggagctgtttcgccgttgtgcctatattgcaaaaacactcaagtggaatccgtccaccgaaggctgtgtaacacaagttttggatacggacattaatacacttttcaatcaacacggggattcgctggctcaactaatatttgaggttatgcgctgtaacgttaccgacgctaagattatattaaaccgcccggtttggcgaacaaccggattcttagatggatgccataatcaatgcttccgtccaatccctacaaaacacgaatataacattgctctatttcgtttaatttgggaacaattatttggcgcccgcgtaactaaaagtacccagacctttccgggaagtactcgtgtgaaaaacctaaaaaaaaaagatctagaaactttacttgattcaattaacgtggatcgttctgcatgtcgtacctaccgccagttgtataacctgcttatgagccatcgccattcgttctcccaacagcgttacaaaattactgcccccgcttgggcacgccacgtgtattttcaagcacatcaaatgcacttggccccgcatgccgaagccatgctacaattagcgctatcggaactgtccccgggatcgtggccgcggataaacggggcggtaaattttgaaagtttataacccattaataccatatatggacatccatagggggggttacataaatactaagcctctgtacaacacaaagggcctctaacaatgcactgaaccacaaccaagctatggacgcaacgcagattaccttggttagagaaagcggacacgtttgtgccgcaagcatatacacatcctggacacagtccggacaattaacacagaacggtctttccgtgttatactacttattatgcaaaaactcatgtgggaaatacgtccctaagtttgccgaaattaccgtacaacaagaggatttatgtcgctactccaggcatggggggagtgtttctgcggcaacgtttgcgtctatctgcagggcggcgtcctcggctgcgttagacgcctggccccttgaaccactgggtaacgcagacacctggcgttgtctccatggcactgccctggccactttacggcgcgtattagggtttaaatcgttttattcgccagtaacattcgagactgatacgaatacaggtcttctgttaaaaacaatccccgatgaacacgcgttgaataatgacaacacgccatctaccggagtattgagggctaattttcccgtggccattgatgtttcagcagtcagcgcatgtaacgcccacacgcaaggtacgtcgctagcctacgcccgcctgaccgcacttaaatctaacggtgacacccagcaacaaacacctttagacgtggaggtaattacaccaaaggcctacatacgtcggaaatataagtctacgttttccccccctatagagcgggaaggtcaaacctccgatttgtttaaccttgaagaacgccgcttggttcttagtggcaatcgcgcaattgtggtaagggtactcttaccgtgttattttgactgtttaacaacggattccaccgttacatcttccctttcaatattagcaacatatagactgtggtacgcggcggcgtttggaaaacccggggttgtccgtccaatctttgcgtatttaggcccggaactcaatccgaagggtgaagacagagactacttttgtactgtcggatttcccggatggaccactcttcggacacaaactccagccgtcgaatctattcgcacggctacggagatgtacatggaaacggatgggttgtggccagtaaccggtattcaggcctttcattatctagccccctggggacagcatccccccttacctccgcgggtgcaggatcttattgggcaaatccctcaagatactggacatgcagatgcaactgtcaattgggacgcgggccggatatctaccgtcttcaaacagcctgtacaactacaagatcgttggatggcaaagtttgatttcagcgccttttttcccacgatatactgcgctatgttccccatgcattttagattaggcaaaatcgtcctggctagaatgcgtcgaggaatggggtgcctaaaacccgcgttggtgtctttttttggggggttacggcacatactcccgagtatatacaaagctattatttttatagccaatgaaattagcctttgcgtcgaacaaacggccttggaacagggctttgctatatgtacttatataaaagatggattttggggaatcttcgccgatttacatacgcgcaatgtatgttcagatcaggcacgttgttcggccttaaatttagcggccgcctgcgaaagagcagtcacgggcttattacgaattcaactaggtcttaactttacacccgccatggaaccggtactccgggtcgagggtgtgtacactcacgcatttacctggtgtaccacgggaagctggctgtggaatttacaaacaaacacgcctccggatttagttggcgtgccatggcgaagtcaggcggcgcgagatttaaaggagcgtctttcaggactcctatgtaccgcaacaaaaattcgagaacggatacaggaaaattgcatatgggaccgtgtcctatacgacatatgggccggacaagttgtggaggctgccagaaaaacatacgtcgatttttttgaacatgtttttgatcgccgttatactccggtatactggagtcttcaggagcaaaattcggaaacaaaagcaataccggcatcttatctgacatacggacacatgcaagataaggattataaaccaagacagataattatggttcgtaatcccaacccacatggacctcctactgttgtttactgggaattgctaccatcgtgtgcctgtattccccccatagactgcgctgctcatctcaagccccttatacacacgtttgtcactattattaaccatcttctagatgctcataatgatttttcaagtccatcattgaaatttactgacgatccccttgcttcatataacttcttgtttttatgacaaaaaaacacgccgcaacaacccatccttaaaataaaaggtttatttactttacaacccgtggtgaatttttatacgtttcaaataactgaacatttttcggtgttaccatggtgcgatttaaccaccaaaaatatacgctcttctgatattccgaatctcgtaaaggtccatttaacaatcccgggggcacttgcaccacaccatctggacaggggggggttccgtggggcaggtcaaaacgctgacccaccccacatgaatatatagcctttataatattgggggccgttccaggctgagggttcagtaacttaacaaacatataatgcggcaatacgcgggtttttgtaaaggggttgttatcaacgacatacattagagtgtttaacaaccataaaactccctcatataaaaaccgacgcattttttccaaaggtcctatttgacactcaacgcgtctaagatatacagacaattgtacaaacagcgatggagatgccccggagggcccaatgccttccagatacattaaaataacacataaggtaaaatctaggacattatccgggcggaatagagtcatccgatagattaacaggcgcggaggcacccccaccgtatacaccctatcttcaaccgcagttaatacggaaaaaataaatccgcggaacgctggttgagtaacacactccatgtagtaacgatcacaggacacctcacttgaatcaccattcaacactactaaaacggtctcttggtgttccggttttacgcgcagtgatacaacagagtttgccaaaaagcgtggcttcaaaccggttacctcccgcgcctcgcatacgaatcttggtattgcttgtattctaagatcttcgatcacgtcgctcacatccaacccctcttcggctcgtgttagtaagttgtcgatcgttacgctgcaacctaaaatgctgggtatatttattccggacatcccatcggccatccccgcgcctccggtttgctcgaattttatccagtaaggtcgaatccgctgcatttaccttgtgtacccgtaacctctcaggggggtgtcctttcataaaatgggataggtttttatatccaacatgcatgtattggttatttattttattgggttccgggattctttcgtcatcttctgtagggtcaggcaaaccccaggaaggacttggtgttctccgtgggccccgttttattacctctgcgcgaacctgcatttcatataatattcggatttgggataaataggactctgttctcgcctttttaaaaatagcctggcataactcttcctctgacctatgtacctcgctttgagttaccaagaatcccaatcgggtggcccgtaatatgaatgaaaaatacggcgcaactagtaatgagattgacgcatttgaatatgatacagaaatttcctggccttgattattgtttacccggtgaagcttaaaacagcgaacaagttcctgtttccatagctcagacaaacgttttatatcatctccataaggggggatataacgagattgaaaactattggcaatatatgcatcatcccctattatgccggtaagatctataacctcgtgatttaaatcggcaatacgtgtttcttctgccattgtaatatgtgaccctttagatggctttatttttaccctctcttcccgtaaccgtttcagctctccttctttgaactggagcctttcggtcagatcgctgttcacatccttgagaccctcaatggttttgaataaattattcacataaccctcgagcatgccgttgatactgttgaccaccgatgttttaaacgcactttgaacgtttgttgttccggacattgcccccccgttaaaggattggttggccttgccaaaccccggttgtgatgtgtccaccgatccacttccttccagaatgtgattgcccgtttcttctagataggaacgtacggtttcggtaatatctccaacatgtctcatgttttttaagttaactattagctttacaagtctagacgcggccgatccagcccgtgttgtatcgttctcgcccattatacgatcaaccgcacgtgtgctgtgagatctatcatcttcattccggcgacctattaacacgcgcaaaggggctgtatttaaaacttggcagacgcgagcatgttcacgtaatgcataacaggccaacacctccccagaaagccgctgtaagggtgagtcaaatactacaccctccccacatacaacgggcggccacacgaccaaacactctcccttcatgcccgttacatcatcctttgccataattaatcttcggttataattataataaagacgcgtcctatcataatccataatagcaacattttgcatacactcaactaggcttgtgacaaccgccgcccctctggccaacgttgcatcggcaacttttaacatctgggacagttctgccgcttgacccatatacgtatttaatggtgcaggggttccattctgttctgatcgtacctttcttacaacgggcacaatacctacacaggctatccagtccacgtatttggcaaaaccgacccttccatttaaaccactggtatagagacaaccggttattccacgcagaaactcaagtaacgatgactgtaatgtttgacgccaggtttcaaaaacctgatgtgcaagccgtacggcttctgattctccacataacccataacgttccgctagagccccggcatgcaggttacattgttggatgtggtggtcccaatctgctgctaggtcctcataccgagttgcatccaacgcgttcatcaaaacggttgcctgaacttggcgaattacagtttccgtagaccgtacagcgctatatatgccttgtccatcggtatatccaaagtcaccggctaggatttttcgaaacaacatactttgcgtggttgggtgtattaacatccagccatcttcctccggaaatgtacaaaaccctatatccggggcgtactcattccagtatatatcgaacatgttcttgtattggtcatttgggttacttccattcaagccctggtcaatagaaacagaacttgctatccttttttcttcactaccggaactgttattaaaaagagacgttatttcggccattgaaaaccacgatgaaaagatcaatttctgtagacagttcttcacccaaaaacgtttttaatccagagacgcccaatggatttgatgacagtgtatatttaaacttcacctctatgcatagcattcaacctatcctctcacggattcgagaacttgccgcaattacgattccaaaagaacgtgttccgcggttgtgttggtttaaacagttactcgaactgcaagcgcctcctgaaatgcagaggaatgagctccccttctccgtttatttaattagcggaaatgccggctccggaaaaagcacgtgtatccaaacgcttaacgaagctatcgattgcattattaccggatccaccagggttgctgcccaaaatgttcatgctaagttatcaacggcttatgcgagtcgtccgataaacacaatctttcatgaatttggttttcgcggaaatcacattcaggctcagctgggccgttacgcatataactggactacgacccccccttctattgaggacctgcaaaaaagagatattgtatactactgggaagttttaattgatataacaaaacgagtgtttcaaatgggggacgacggtcgcggaggaacatcgacatttaaaaccctgtgggcaattgaacgtttgcttaataaacctacaggctcaatgtccggaaccgcgtttatcgcatgcggttcccttccggcttttacccggagcaacgttattgttattgatgaagcaggattgctagggcgtcatattctcacggccgttgtttactgttggtggcttttgaatgctatatatcaaagccctcagtacataaacggtcgaaaaccggtcatagtatgcgtcggttcgcccacccaaactgactcgttagaatctcattttcaacatgacatgcagcgttcacacgtaactcctagtgaaaatatactcacgtatataatctgcaatcaaactctgcgtcaatatactaacatctcacataactgggcaatctttattaataacaaacgatgtcaagaggacgattttggaaatcttttaaaaacgcttgagtacgggctacctattaccgaagcacatgcgcgtctggtcgatacatttgttgtacctgcatcctatattaacaatcctgctaatctccccggatggacgcgtctgtattcgtcgcataaggaggtgagcgcgtatatgagtaagttacacgcgcatttaaaactatcgaaaaatgaccatttttctgtgtttgccttaccgacttatacattcatccggctaacggcatttgatgaataccgcaaattaacgggacaacccggactttctgttgaacattggatacgggcaaactccggtcgtttgcacaattattcccaaagccgagatcatgacatgggaacagttaaatacgaaacacattcaaatcgcgacttaattgtagcccgtacagacatcacttacgtgctaaatagtctcgtagttgtaaccacaagactacgtaagttagttattggattcagtggtacatttcaatcgtttgcaaaggttttacgtgacgactcctttgtgaaggctcgaggagagacatccattgaatatgcttaccggtttctgtcaaacctaatctttggaggcttgattaacttttacaattttttgttaaataaaaacctacatcccgataaggtatcgttagcatacaaacggttagctgccttaaccctggagttattgtctggaacaaacaaagcccccttacacgaagcagcggttaatggggcgggtgccgggattgaccgtgatggtgcagctacttctgccgataaagccttctgctttaccaaagcccccgagtccaaagtaacggcctccatacccgaagacccggatgatgtaatttttacggcacttaacgacgaggttattgacttggtatactgccagtacgaattttcctatcccaaatcatccaatgaggtccatgctcagtttctgttaatgaaagctatttacgatggtcgatatgccatattagcagagcttttcgaaagcagctttacaaccgccccctttagcgcgtatgtcgataatgttaatttcaacggaagcgagcttttgatcggcaatgtgcggggggggctgttatctttggcattacaaacagatacgtatacccttttggggtatacttttgcacccgtgccagtctttgtagaggaactgacccgaaaaaagctgtaccgcgaaactaccgaaatgttatatgctctacacgtacctcttatggtcttacaggatcaacatgggtttgtgtccatcgtaaacgctaacgtatgtgaatttaccgagtctatagaggatgcagaattggcaatggccaccacggtggactatggccttagttctaaactagccatgacaattgcacgctcacagggcctgagtttagagaaggtagctatctgttttacggcggataaactgcgcctaaatagtgtgtatgttgccatgtcgcgtacggtctcctctaggttcttaaaaatgaatctaaaccctctacgggaacgatatgaaaaatccgcagaaattagcgatcacattcttgccgctctacgtgatcccaacgtacacgttgtgtattaaagcattgtataaaaacacgcatgcgggcttgctgttctcatttctaggttttgtcttaaatacacccgccatgagcatctctggacccccaacgacgtttattttatataggttacatggggtcaggcgggttcttcactggactttaccggatcatgaacaaaccctctacgcatttacgggtgggtcaagatcaatggcggtgaagacggacgctcgatgtgatacaatgagcggtggtatgatcgtccttcaacacacccatacagtgaccctgctaaccatagactgttctactgacttttcatcatacgcatttacgcaccgggatttccacttacaggacaaaccccacgcaacatttgcgatgccgtttatgtcctgggtcggttctgacccaacatctcagctgtacagtaatgtggggggggtactatccgtaataacggaagatgacctatccatgtgtatctcaattgttatatacggtttacgggtaaacagacctgacgatcagaccacaccaacaccaaccccgcaccagtatacatcgcaaaggcggcagcctgaaaccaactgtccttcaccacaaccggcctttttcacatcagacgacgacgttctttcgttaatattacgggacgccgcaaacgcgtaaagacagattcaagactaacatttatcccaactgattacatttcatacgcgaataaacgacacaaaaaatttatatttaacggcttttaatttgaagacacctatcctcttaacgttgaggagccttgcaggttgggtgccgcgcttcaccggtattatacataaccgatttaccgtgtttacggcagtctgaccatttaccagtgtatgtctgtaatacgacgttgttgtgtcccgacaaaattaactcgcgtacaaatttctgatgttcccccggcgtggcaacgctggcatttccaaacacattacgttctcgtacgtccatgaccgctattttcagtattaattggttggtcggtcaaagtattttccttatgtaaaaggacacgatctaaagccgtaaactcgtacacaaacactggtaccaacggacgcgattttccgtccgttgagcgggtgtaatatcggcgaggtcttcttgcacgaatactctcgtacagtaggtttctgacacggggtgcatgggttttttgacacaacacaaacatttgcaggctcttatgactggatggattgaatttatttttagatagggtcacgtgtttttgtcgtgacacgcctcgaccagaaaaggctgcggttttcgtacacgcgaccgttatttcacaggcgttcataatcaagctgcggcggatggtgtcggttaattgtctccgcccaagttcgtcaatagatgataccatgaacaacgtatcaaatggtacatagtcgtctttggttttctcaatacagcccgcgtgcccaatcggaaaattttcatttgcatcaacgctattttctgtaaaatcggtctgaacactgtgttggctggctacctgtttaaaatttgggatcgaacacggtccacgatgcaatccccaaccccattgaagcaatgccgtcggtacggaaggaggcaactccgaaaacattatggtacgcgagagggtcgattggagtgttatataacactccaatcgatctcgggttcgcctttacgcgtaaaatactcattggcttgaacgaaatgtcgacaattccgaaatggaacacgggacaatggcgacggatgcgcgtgtgttagcaccagatgacatcttgaattcggttgggttgtcttctgtgcatgcgcaccccacagcataaaaactaaccctgtacggttctcgcataacctctgtagcacgcgttgcaccagccgcccccagcctaagtatacatgcgaccccggagtcccgcgacgaaccgtaagcgtggtattcagcaataacaccccctgccttgcccaactctccaggcatccgtgagtgggcggagtcatatttgggtatgattccatgagggccgcaaaaatatttttaagactagacggtggtgttatgccacgttttacactaaacgctagcccatgtgcatgtcccgcggtagggtatggatcttgaccaataattacaacgcgaatgctctggggtccgcaaaatcgcgtccatgcaaaaatatcgcctgtagatggaagtatttcttcccctgaatttaaaagacgattgtattctaaaaaaatacctttcgcgtacggctctttaagttcgtccgacaacaggtcataccactcaggggaaatgttaaacttgctgaaaacttcaaccgaatccagttgcgaagagacgggggtgaacgtttccgtgtcgtaatgatgtgacatgttatttaacttgaaggttggggggtctagcttaacccccaaaggcggcccgcggggtcgcttgcgggtttttttggtaaccggatgggccaaaacataaatgtcctttgaatccgatagtttcatttcattggcatacgcgttggaacaaacggtcggctccccagacacatccattttccgggatatttgtggaagatggagtagagtctacccatacaccggaaagggcatccaacaaagcatcgcgtatgtccccgcttttatgttcttcaccaacagattgtgtcagcccctttaaggtgacgtatggatttgtccagtacgccatttgtttgtctttaaaccaaagtataacttccggtactggacattttgtcttaaccacgattcccgatagcgcctcgctgaggtttgataccgggggtgccgcatagtcccacgcctcatataccgatgacacgcacggttccgttataatcaaactcacatccgatagcggtttggctccaaaaaacaacggagtgtcgtcttggagatgaagacaatacgcgattgtgatagtttttaaaaaaactatcatctgcagtaaccatttatgtgatgccatgacgcttgtgttttcccttcactacgacgttgtcgtatcctttgaaaaacttgaccactctaatggaagcatggacaagtatgagttttatatatacagttggcctttagttaaactcttggtgtcatatctcattttcctaaaaagggcgatcttaatatgtcaaacgtcacggcgtgccgacaaagcgaatttccatgcaagatttggatgtagtatttatacacccaatcacacgtcacgtattaagctttacagtcccccgttatctgatataatcacttttcttaacacgtcatcgggaaaacagatgtttatattatacctctcgcggtcatttatggcaaatacttagaccgttttcaagcggactgaaaacgctcaaattgccttttggaggcctgcccaacggccattatcccttggatctaagattgatttgcggtaacgtttgccaatcaagctttaaaaacgtaccccaaacttaaaacgctcaaattgccttttggaggcctgcccaacggccattatcccttggatctaagattgatttgcggtaacgtttgccaaacccacgcatttcagtttaaatatttctaagcattcttagtgcgtacttggcagcgtgcttaaaatatcaaccaatatccattatgctaaacgtttccttctatccgtttcaatccattaaaagtccattaccaaaaatgatgcatcatacctaattcacctaaaaacctgactcattgcagcagcgtttcctccttgcagactatccagttggcattttaaacgggtccggctgcctgaaccgaaaacaccgttgcctttactgtaagtacaaaactaaaatttatatttgcgtgcgtattttgtaacatatatgccttttatccccccgcaagtttgctttaccctcgccttcaccacccccgccaccttccggccattgtaataactttaattgctataagacatacccaaaccggatgatttttgccgctggaaaaacagcttctaattttcccgtctcaactcggccttggttgcatctccaagtatacctttagtttgctcccgtagaggtgtataaatacaaacggtgacaagtattgagcgtaatctcaaatttttgtaatttagggcggagcgcttacgacagcacatgcgtactgttagactgttatgtttattgtatttgcagagcaggatgccccggttactccgagaccggattgcgggcattccgaatcgtgtacggacttaccagggggcagtatttacaccttgggttccagatataccaacccttacgaccaatagcaacactcaggtatttttaaaatgcacgtttaatgatcataatttacatacagttggtaataaagcagaccgtggatgtttaaggcatttccttccccctcccaacaaactaggacttcttcatcttgtttggaatacctttacccgctttaccggcagagctttttttggtaaggtgtttcagtgaacctgatgttgatccggaggtggagggggtattggactccccctgtggagaggcaactttgcgggttttacttcccttacatgccgaatcagactcagatgtcaggtctattgttaagcatcgtttaacgtctctgccggtatgaaataaacggcgcttagcaccccttgcgcttcccggtttaatccccggtaacacagaaaaaagcctgactttttggggtgtatttaccaatcgggtatccctttcatcgccacgagaggtctccccggttgaggtggtttctggtcttacaattggacctgtaattagttggatggctgtatctttccaggtccaggtttgcatggttaggcgggttggatcggtacatcgatccaacaagaataacatgtttgttacaaacggtcctgttgaatcatgcaaaagacaacgcagggatgtttttaatcccgcctcatcacgcccgtaaatacctatatagtttaatatcaacatttttgtaggctctacaatttcgggttgatacagttccgcaagttgatcatcaagccatccgagtaaaggttgcatgtaacacgggaatctcgcgtttccctctgttcctctatccgtggctcgaaaaggcagtctgtccatggttcgtgggtcttgattaattcccacagatactggacgatcacggtagtcctgccccccggtccggggttgctgtgcagattcaatcgagccatacaccaccggggtcgccgatcgaacagcaggttggtctttaaaaaataccttccgtaaaaatgatgcggtagagcatgttttggttacaccagggctcgagtctcgggtcggtggttgtatagaatcctgttgagagtcacttggtgactctgctgtgggctctctagccgacgattgaaggggcccagggtttggtgattgaatgggctcccgactcgatcttgatgttggctgttggatggactcccgactcggtcctgggcttggtggcagaagatctatgacatctcccggtaggatgtcgatggaatcttcaaatgacggctcagaaaaaccatcgtcgtcggatgggtgcacttcatattccttgtaacttgtatcacttacgatcttatgcaggatggattgcactggacaccggcagagaggacactggacgctggtggaggtccatgcccgaatacaaacaaagcagaagtcgtgcaaacacggcatggtttttccgagatcggaaacggtgctcatgcatatggtgcaggtattatccgaagcgtcggaggtgccgctaccgcccgctaatatggtatccatggtaacaactggctgtattctaatgtccgggcatccaaacacgtagcagaactgccatgcgttctaaattgtgagttgtggcgagtacatttttataattggtaccaacgaagacacacccctatatccctccacccatttcttttaagtcccacccactaaaacgtgggtataaaatgtgtattggggtaggcggacagtcccaacaaacagggaagttgattggtataaccttgggccgggtatacagctaagtgacattttagattctgtctttatttagataaagagcgatacgaagacatttctccacccccctgtaatacccgtaaataaaggtaagtccacaaacaaaagcactgtatataggaagtcgggtgtattgggacagttactccattagaggcgtacagacaatactgggatagggtaatgcaagtcccccccgatggtcgccccgcaaacgcgcggggaggtggggtcgcttttttttttctctctcgagggggccgcgagagggctgggcccccccccccggggtccgccgggcgcccagaaaccggggggggttattttcggggggggtccgaccagcccgcccgtcgcccgcccgcacagacagacagacacttttttcataaaaaccgttccgcttttattaacaaacaacaaacagtccgcgcgccggtggcgctcacgagaaaaggaggggactccgtcacccccgactctgcggggggctcctccccccgcgccctccccacacatcgtcctcgtcctcggaggacgaggacgaggacaacagctccaccttgaccgccgggcgcgagcccacccggcggtctcgcagcacacccggggccaccgacacgacgctcaccccaaaggatgaccccggtgcgtccccgtcgtccccgcccccctcctcgctgtcccacgcgtcttcacaccccacctcccaatcgtccggctccaaagcgtgttctctgtcgtctgcggtgcgccgctgtcgccccgcctgggtttctgccggccgttccgagcccccgtggtgtccgaacgcgaaccgtgttccgtcgctcccctccaacaccgtctccgcggccccaaaaccgggcggccacattactctgggaatcggggggagggcattccgagcctcgtccgccgacgcatacagcgccaccgaccgaccggccacgggtggaagcacgagtggttccgcggcagggtcgggttccagcagggcgtggcggcaaaacaccctcgcccaggtgggtacgtcgccggcctccggcccggcggcccccggtctccgtccctcgggaaggaagacgggtcgaagcgcggcacccaggccccatcggtttgctgcgcggtggctatgtgccgcctcgtccacaaagtcggctgccccgagccccagaccccgagactgtcgcgcgaggtccttgcaaccgtcaaaacccggcagcacgtactgccggtattcacggggcgacagggggacgcgggtcttggggcccgcgcgggtacacacggtgtatgcgacgttcccaccgcggcacaaacacaggggttgttcgcccgggtacaggttggcaaacgcagtctcgatacgagcaaaactcgctggcccaaaggtgcgcgacgatgcaaacacggcccgggcgagtccttctgtgaccgccgagtctggccatcggacgacggcctgggcgtccggtcgcgccggggcccggacgtacacgtgatactgagacaaagcgggtccatccccgggccacctctcgagggccaccgcgtccaacaccagcaaccggcgccgggcagaggccaaccgcgagcctagatactcgacggccccggcaaaggccaggtctcgggtcgacagtaataaaacgccccgggcgttcaaagcggacacgtccggcgggccggtccagttcccggcccaggcatgagtgctcggcaggcacaaccggttactcagggctgccaggaccacagacagtccccctcgggatggactccatgacggtcccggatctgtcgcgagggtgctctcgagggggccgttgatgtcctctccgggcaacggatcgtagatgatcagaagcctcacatcctccgggtctgggatctgccgcatccaggcgcacctccgtcgcagcgcctccactccgctgggtggaccaaaccgtcggtctcctccgcccggacgccgagcggcgatttccgccaaggcgccgggatcaaagcttagcgcagggcgccaggccgtgggaaacaatgggtcgtcgaccagacgggcgatggtttcgggggtacagtacgccttgcgagcctggtccgacgggaccggggtatgcagggccccccggggaatacgccgaaatcccccgtttggggccggtccgtcaagtggcatcgttattacggcggggggatccaccacagggcccgaggtgatggtcacgggctcggatacccgcctcttggccttggaaaccacatgatcgtctgcaacccgggcgtccgcgacgggtgtctccctaatcttgtcgaggaggcttctgctctcgactggctgggacttgcgcttgcgcggagttcgtaaacgatcatccggtggacacacagaaagagagcgcgcggcggccgacggctgagggtcgggagtctgtgtggccggggttgttggagaagggtgaccgcgggagatccgcgccgccggactggagcccgttgcctcggggtatgccatgccggcaaaggctctgcggagactctgtaggataaagtgtttttgggcccggtcgtaccgacggctcatagccacggccgcggccgcgtgggggagagcccagagggcctcccccgtggccatggcttcgcctacatgcggaacgggagacgctacgctccccgtaacggcggtacccgcccgtcccggtggcaacagcttttggtagaactggttcagggccgagttgacaccggtcagcttggggttctggagccatgctatagggtctctgtctggacagtagatcaggttaatcagcgcgcggtactgtctagccggatctcccaactccggcacgtaaagcggcgcgggttcagttgaggcctcgtaacgagcccgcgccgctctcacagcctcatcctcccagtgaccctctctggtctccccggacggtccaaaccgcaccctgttggatgggaggggcgccgatccgggccaagggcttccgtcgggcatcatgagcggccccgacaccgggggaattatcggggttctggatcgcggcagggaaaatgatttctgtctctggcgccccggttcccccgcaagacgtttggtcttacgaatcctcggatcgggaccgctgatggatcgatatcccggttggatattttgtttcgtcgacccaccatcatttgagtccgaatcatccgaatttgacggggaaggggcgtgttcgcgtccggacctgctgcctgtagtttcacttcccaccgaaacgcgccggggttcatcgtcttcatcctccgatgacgatccccacgacgaggaagaggatgaagacgaaacaaactcacgactctttggctttttctccaccgggctgtcatcctcaatcgggtctggtgcgtgggatcttcccggcagggccaaaaacgctctaggtttgccccccgacgaacgtccagggacgcgaggtgttataccccgggcatcatgtttccttgggcgggtatcatcggtctcaaacggcaggtccgcctttgcccccttagcgggaacgctgtccgaaaggacgtggtacaattgctcaaccgggccgggtacaggtccaccgggtttccgcgccgggagtgggaccttaaccttcaaagtctttttcttcgggctctttccctgagcgggccgttgagttttctggagaactactccgtcccccgatgcatgcgcatgacccgcttgctcatcgcccggctttttacccgagatggactgagtttgtctgtctcgatggaccaccgacggcaaacctggtgaatttcctctcgtcgtttgtcggggtatagaccgctggtcttcccgttgatcgttcccggcggcgtctccaacaggagacgcgggggatacaggggagaaggcctgcgggaacggaggggtcgtacctctgcccgtttccccatcgttcatcggtggttttggagacctagcaagcttcgttccgagagagactgtctcgagggagcgatcggctcctgttggttctcgcgcgccggcctccgagaatcgggtgtggaagacctcggccagcgggattacaggcgagcccgttagatcctgaccgtcctcgcatacgtagtcgtcttgtgttagctcttcgccaacatcttccgttctgggttctggttgaagtcccgatacggagggaattgaaacgatctcatgttcccgtcccaccatgaccccgttctctccaaatagtagatcgtcaggctgactcgaggtgaccacccgggccctgtgttcggcggccgcggcggccgcgtccaacaggtccattaactccaaagtatcaggcgaccccgcgcgttggggtgtagagcgctgcatcggcggcgtatccatcgcgctggggtgaatttagacgtacccgagttttccaaacgctctcgcagccttcaaaggattgcggttgcggttggtgagggagttccaacagtacttaaaacgtgttgtgcccccccctcgaccgcatatttcctccccgtgtcgtcaccgtgtaaatattcttaatgataagacgatgtagtgattggacgagactcgaggcgggaagttcatggaccatagtatgcgtttaaggagagaccgctggttggcgatgtacgcccggtgtctatttccgcataccttacaacatcataacaagggataccagacatgtgaatttcatttacatatgtttaaataacaaccaatcatcgtgtgtctacagacgatatataatatacataaacacaattggggttgtctcacacgcaaaacatcttatataacacgggttgtttccacccatccggcatctagttaatcaaatgcacgtcgacggtgtgtttgggtccctctccgtcgtcattacgttcgctcaatcaacaagcgtatacaccaccacccctcccaacgattatcatgtcaggcggcacgaagcccgcgataacccataaaatacacacggggttgtggtgttcacgtaaccccccgccgatggggagggggcgcggtaccccgccgatggggagggggcgcggtaccccgccgatggggagggggcgcggtaccccgccgatgtttataaccataattctctaaaccgttgtagaaaatcacaaaaaaatttattcaaaaacaagtcgaagaacttcatatctgaggcatgtaaacccattcgcacttcctggggtggaatggggtggggtgggggggtgaaaaaggggggggttaaattgggcgtccgcatgtctgtggtgtacgccaatcggatacactcttttaatctgcattcgcacttcccgttttttcactgtatgggttttcatgttttggcatgtgtccaaccaccgttcgcactttctttctatatatatatatatatatatatagagagagagagagagagagagggagagagtttcttgttcgcgcgtgttcccgcgatgtcgcggttttatggggtgtgggcgggcttttcacagaatatatatattccaaatggagcggcaggctttttaaaatcgatttgacgtgataaaaaaaacacacgggcccccctttttttggtgttataaaggcaacccaatcgaaggtctcccgccccggaatcccccattgccattttacccaagtagccttattcatagatgtaaacgtttgggtgtgtgttttgttgtgcagggttcgtccgattcataacgcgacagcgtcgagtcggttttaagggaaaaggttactacggccccaaggacatgttttgcacctcaccggctacgcggggcgactcgtccgagtcaaaacccggggcatcggttgatgttaacggaaagatggaatatggatctgcaccaggacccctgaacggccgggatacgtcgcggggccccggcgcgttttgtactccgggttgggagatccacccggccaggctcgttgaggacatcaaccgtgtttttttatgtattgcacagtcgtcgggacgcgtcacgcgagattcacgaagattgcggcgcatatgcctcgacttttatctaatgggtcgcaccagacagcgtcccacgttagcgtgctgggaggaattgttacagcttcaacccacccagacgcagtgcttacgcgctactttaatggaagtgtcccatcgaccccctcggggggaagacgggttcattgaggcgccgaatgttcctttgcataggagcgcactggaatgtgacgtatctgatgatggtggtgaagacgatagcgacgatgatgggtctacgccatcggatgtaattgaatttcgggattccgacgcggaatcatcggacggggaagactttatagtggaagaagaatcagaggagagcaccgattcttgtgaaccagacggggtacccggcgattgttatcgagacggggatgggtgcaacaccccgtccccaaagagaccccagcgtgccatcgagcgatacgcgggtgcagaaaccgcggaatatacggccgcgaaagcgctcaccgcgttgggcgaggggggtgtagattggaagcgacgtcgacacgaagccccgcgccggcatgatataccgcccccccatggcgtgtagtctttataaataaatacaatggtttggctcgtgtctttttttgatgtctgtctgtgggggagtggggtgttgtggatattagagggtagagggtgctggtttgaacgtctccattaacccacggggtccccacacgggccgtgtggtatgaatctctgcggatcccgcggtgagcacccgggcggtgaatatgccggactttactgcacacgacacgatacccccgcgcaccgggctctcatgaacgacgccgaacggtacttcgccgccgcgctatgcgccatatctaccgaggcctacgaggcttttatacacagcccctccgagagaccgtgcgcgagtttgtgggggagggcaaaggacgccttcggacggatgtgcggggagctcgcagcggatagacaacgtccaccctcggttccgccgatccgcagagcggtgttatcgttattacgcgagcaatgcatgccggatccacaatcgcatctggagctcagcgagcggctgatattgatggcatattggtgctgtttgggacacgccggacttccgactattggattgtcgcccgataataaatgcatccgcgccgaattatatgaccgccccgggggaatttgtcacaggctttttgacgcgtacctgggctgcgggtcccttggagtcccaagaacccacgagagatcctgacaccccatccctttatatagaaaaaaaaaaaaataaatttaaaacatacaccggataaaagcgtactgttttttatttaaatttacacgctcgacgttgccccggttcggtgatcaccgggtcttatctatatacaccgtgtaactcgaacccccgtgactccctccaatcgcgttaccaaactcttcttccgtatccgtagattccgagtcctcgaaatcgtccacttatccaacaaattgtgacgttatatatcccaaggcaaaggccgctcccgtcatagcaaatacaaagacaattattagcgtaatataacagaattttttacgatgatatattttatgttgatattttccaattcgacgcaaaaattcatctgccgtttcattttcgctatcactataataacacttttcagccgaacggctcggttgtatggctgttatcgttgtattatttggttgcgctcgcggggttaccaccgcttccatcagtaaggccacggcctcaccctccatggtgttttgtccggccatagaaatccagattgtaaggccagcaggctagtttaaaagtgtttaataccacaccttttgatatttatatacatgcaagattctagattattcatcaataggtcgtttaaagcgcgttttcataaacgttgtcagctataccgacattctcacaaagaggtaaagttaccttacgttattattaaataaaacatgtagacattattaataatcctaggaacaatcaaatccatatttgtaagttatgtttaacccctcccctttttgtcattatctccgccctcttataatcggatcactttataagtgtgtcggtgagtatattttgtacagttgttggacaacaggtttttggttcattaacactatcaacataagtcggggtatacaagtataatgaacgacgttgatgcaacagacacctttgttggacaaggaaagttccgtggcgccatctcaacatcaccgtcacatattatgcaaacatgtgggtttatacaacagatgtttccagttgaaatgtcgcccggcatagaatctgaggatgatcccaattatgacgttaacatggatatacagtcttttaatatatttgatggtgtacacgaaactgaagccgaagcctctgtggcattgtgcgcagaagcacgcgttggaattaataaagcgggatttgtaatattaaaaacgtttacaccaggggcggaaggttttgcgtttgcgtgtatggacagtaaaacatgtgaacatgtggtcattaaagcgggtcaacgtcaaggaacggccaccgaggcaaccgtgttaagagcgttaacccacccatccgttgtacagcttaaaggaacgtttacgtataacaaaatgacatgtcttatattaccacgttaccgaacagatttatactgctatctagctgcaaagcgcaacctccccatatgtgacattttagcaattcagcgatctgtattacgcgcgttacagtatcttcataataacagtattattcaccgtgatataaaatctgaaaatatatttattaaccacccaggtgatgtttgtgtgggagactttggagcagcgtgtttccccgtggatattaatgccaacaggtattatggctgggctggaacaatcgccacaaactctcctgagttattggctagagatccatatggacctgccgtggacatatggagtgccgggattgtattatttgaaatggctacaggacagaactcgttatttgaacgagacggtttagatggcaattgtgacagtgagcgtcaaattaaacttattatacgacgatctggaactcatcccaatgaatttcccattaaccctacatcaaatcttcgtcgacaatacattggtttggcaaaacggtcttctcgaaaacccggatccaggccattgtggacaaatctatatgagttgccaattgatttggagtatttgatatgtaagatgttatcgtttgacgcacgtcatcgaccatcggcagaggtgttgcttaaccactctgttttccaaactcttcccgatccatatccaaatccaatggaagttggagattaaaattcattaagcctgttaataaaatattgtataaattgtgtttataacgtataacccgttaaggcaaatagggtacaaacgcgcaatgttttgaaatactaatataaataacataaccaatagaaacttaatacagagtcacgccccattacaacaaggataaaacacgggatcattttcttaacattgtagtagcgctgaaaagcgtcccctcccccggctcacagagctgctcttcggtgtagttgggtatactggtgcgcctcatttaatcgcgatgtttttaatccaatgtttgatatcggccgttatattttacatacaagtgaccaacgctttgatcttcaagggcgaccacgtgagcttgcaagttaacagcagtctcacgtctatccttattcccatgcaaaatgataattatacagagataaaaggacagcttgtctttattggagagcaactacctaccgggacaaactatagcggaacactggaactgttatacgcggatacggtggcgttttgtttccggtcagtacaagtaataagatacgacggatgtccccggattagaacgagcgcttttatttcgtgtaggtacaaacattcgtggcattatggtaactcaacggatcggatatcaacagagccggatgctggtgtaatgttgaaaattaccaaaccgggaataaatgatgctggtgtgtatgtacttcttgttcggttagaccatagcagatccaccgatggtttcattcttggtgtaaatgtatatacagcgggctcgcatcacaacattcacggggttatctacacttctccatctctacagaatggatattctacaagagccctttttcaacaagctcgtttgtgtgatttacccgcgacacccaaagggtccggtacctccctgtttcaacatatgcttgatcttcgtgccggtaaatcgttagaggataacccttggttacatgaggacgttgttacgacagaaactaagtccgttgttaaggaggggatagaaaatcacgtatatccaacggatatgtccacgttacccgaaaagtcccttaatgatcctccagaaaatctacttataattattcctatagtagcgtctgtcatgatcctcaccgccatggttattgttattgtaataagcgttaagcgacgtagaattaaaaaacatccaatttatcgcccaaatacaaaaacaagaaggggcatacaaaatgcgacaccagaatccgatgtgatgttggaggccgccattgcacaactagcaacgattcgcgaagaatcccccccacattccgttgtaaacccgtttgttaaatagaactaattatcccggattttatattaaataaactatatgcgttttatttagcgttttgattacgcgttgtgatatgaggggaaggattaagaatctcctaactataagttaacacgcccacatttgggcggggatgttttatgaagccttaaaggccgagctggtatacacgagagcagtccatggttttagacctcgggcgaattgcgtggttttaagtgactatattccgagggtcgcctgtaatatggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgtttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttctacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtgatcaccgaaccggggcaacgtcgagcgtgtaaatttaaataaaaaacagtacgcttttatccggtgtatgttttaaatttatttttttttttttctatataaagggatggggtgtcaggatctctcgtgggttcttgggactccaagggacccgcagcccaggtacgcgtcaaaaagcctgtgacaaattcccccggggcggtcatataattcggcgcggatgcatttattatcgggcgacaatccaatagtcggaagtccggcgtgtcccaaacagcaccaatatgccatcaatatcagccgctcgctgagctccagatgcgattgtggatccggcatgcattgctcgcgtaataacgataacaccgctctgcggatcggcggaaccgagggtggacgttgtctatccgctgcgagctccccgcacatccgtccgaaggcgtcctttgccctcccccacaaactcgcgcacggtctctcggaggggctgtgtataaaagcctcgtaggcctcggtagatatggcgcatagcgcggcggcgaagtaccgttcggcgtcgttcatgagagcccggtgcgcgggggtatcgtgtcgtgtgcagtaaagtccggcatattcaccgcccgggtgctcaccgcgggatccgcagagattcataccacacggcccgtgtggggaccccgtgggttaatggagacgttcaaaccagcaccctctaccctctaatatccacaacaccccactcccccacagacagacatcaaaaaaagacacgagccaaaccattgtatttatttataaagactacacgccatgggggggcggtatatcatgccggcgcggggcttcgtgtcgacgtcgcttccaatctacacccccctcgcccaacgcggtgagcgctttcgcggccgtatattccgcggtttctgcacccgcgtatcgctcgatggcacgctggggtctctttggggacggggtgttgcacccatccccgtctcgataacaatcgccgggtaccccgtctggttcacaagaatcggtgctctcctctgattcttcttccactataaagtcttccccgtccgatgattccgcgtcggaatcccgaaattcaattacatccgatggcgtagacccatcatcgtcgctatcgtcttcaccaccatcatcagatacgtcacattccagtgcgctcctatgcaaaggaacattcggcgcctcaatgaacccgtcttccccccgagggggtcgatgggacacttccattaaagtagcgcgtaagcactgcgtctgggtgggttgaagctgtaacaattcctcccagcacgctaacgtgggacgctgtctggtgcgacccattagataaaagtcgaggcatatgcgccgcaatcttcgtgaatctcgcgtgacgcgtcccgacgactgtgcaatacataaaaaaacacggttgatgtcctcaacgagcctggccgggtggatctcccaacccggagtacaaaacgcgccggggccccgcgacgtatcccggccgttcaggggtcctggtgcagatccatattccatctttccgttaacatcaaccgatgccccgggttttgactcggacgagtcgccccgcgtagccggtgaggtgcaaaacatgtccttggggccgtagtaaccttttcccttaaaaccgactcgacgctgtcgcgttatgaatcggacgaaccctgcacaacaaaacacacacccaaacgtttacatctatgaataaggctacttgggtaaaatggcaatgggggattccggggcgggagaccttcgattgggttgcctttataacaccaaaaaaaggggggcccgtgtgttttttttatcacgtcaaatcgattttaaaaagcctgccgctccatttggaatatatatattctgtgaaaagcccgcccacaccccataaaaccgcgacatcgcgggaacacgcgcgaacaagaaactctctccctctctctctctctctctctctatatatatatatatatatatatagaaagaaagtgcgaacggtggttggacacatgccaaaacatgaaaacccatacagtgaaaaaacgggaagtgcgaatgcagattaaaagagtgtatccgattggcgtacaccacagacatgcggacgcccaatttaacccccccctttttcacccccccaccccaccccattccaccccaggaagtgcgaatgggtttacatgcctcagatatgaagttcttcgacttgtttttgaataaatttttttgtgattttctacaacggtttagagaattatggttataaacatcggcggggtaccgcgccccctccccatcggcggggtaccgcgccccctccccatcggcggggtaccgcgccccctccccatcggcggggggttacgtgaacaccacaaccccgtgtgtattttatgggttatcgcgggcttcgtgccgcctgacatgataatcgttgggaggggtggtggtgtatacgcttgttgattgagcgaacgtaatgacgacggagagggacccaaacacaccgtcgacgtgcatttgattaactagatgccggatgggtggaaacaacccgtgttatataagatgttttgcgtgtgagacaaccccaattgtgtttatgtatattatatatcgtctgtagacacacgatgattggttgttatttaaacatatgtaaatgaaattcacatgtctggtatcccttgttatgatgttgtaaggtatgcggaaatagacaccgggcgtacatcgccaaccagcggtctctccttaaacgcatactatggtccatgaacttcccgcctcgagtctcgtccaatcactacatcgtcttatcattaagaatatttacacggtgacgacacggggaggaaatatgcggtcgaggggggggcacaacacgttttaagtactgttggaactccctcaccaaccgcaaccgcaatcctttgaaggctgcgagagcgtttggaaaactcgggtacgtctaaattcaccccagcgcgatggatacgccgccgatgcagcgctctacaccccaacgcgcggggtcgcctgatactttggagttaatggacctgttggacgcggccgccgcggccgccgaacacagggcccgggtggtcacctcgagtcagcctgacgatctactatttggagagaacggggtcatggtgggacgggaacatgagatcgtttcaattccctccgtatcgggacttcaaccagaacccagaacggaagatgttggcgaagagctaacacaagacgactacgtatgcgaggacggtcaggatctaacgggctcgcctgtaatcccgctggccgaggtcttccacacccgattctcggaggccggcgcgcgagaaccaacaggagccgatcgctccctcgagacagtctctctcggaacgaagcttgctaggtctccaaaaccaccgatgaacgatggggaaacgggcagaggtacgacccctccgttcccgcaggccttctcccctgtatcccccgcgtctcctgttggagacgccgccgggaacgatcaacgggaagaccagcggtctataccccgacaaacgacgagaggaaattcaccaggtttgccgtcggtggtccatcgagacagacaaactcagtccatctcgggtaaaaagccgggcgatgagcaagcgggtcatgcgcatgcatcgggggacggagtagttctccagaaaactcaacggcccgctcagggaaagagcccgaagaaaaagactttgaaggttaaggtcccactcccggcgcggaaacccggtggacctgtacccggcccggttgagcaattgtaccacgtcctttcggacagcgttcccgctaagggggcaaaggcggacctgccgtttgagaccgatgatacccgcccaaggaaacatgatgcccggggtataacacctcgcgtccctggacgttcgtcggggggcaaacctagagcgtttttggccctgccgggaagatcccacgcaccagacccgattgaggatgacagcccggtggagaaaaagccaaagagtcgtgagtttgtttcgtcttcatcctcttcctcgtcgtggggatcgtcatcggaggatgaagacgatgaaccccggcgcgtttcggtgggaagtgaaactacaggcagcaggtccggacgcgaacacgccccttccccgtcaaattcggatgattcggactcaaatgatggtgggtcgacgaaacaaaatatccaaccgggatatcgatccatcagcggtcccgatccgaggattcgtaagaccaaacgtcttgcgggggaaccggggcgccagagacagaaatcattttccctgccgcgatccagaaccccgataattcccccggtgtcggggccgctcatgatgcccgacggaagcccttggcccggatcggcgcccctcccatccaacagggtgcggtttggaccgtccggggagaccagagagggtcactgggaggatgaggctgtgagagcggcgcgggctcgttacgaggcctcaactgaacccgcgccgctttacgtgccggagttgggagatccggctagacagtaccgcgcgctgattaacctgatctactgtccagacagagaccctatagcatggctccagaaccccaagctgaccggtgtcaactcggccctgaaccagttctaccaaaagctgttgccaccgggacgggcgggtaccgccgttacggggagcgtagcgtctcccgttccgcatgtaggcgaagccatggccacgggggaggccctctgggctctcccccacgcggccgcggccgtggctatgagccgtcggtacgaccgggcccaaaaacactttatcctacagagtctccgcagagcctttgccggcatggcataccccgaggcaacgggctccagtccggcggcgcggatctcccgcggtcacccttctccaacaaccccggccacacagactcccgaccctcagccgtcggccgccgcgcgctctctttctgtgtgtccaccggatgatcgtttacgaactccgcgcaagcgcaagtcccagccagtcgagagcagaagcctcctcgacaagattagggagacacccgtcgcggacgcccgggttgcagacgatcatgtggtttccaaggccaagaggcgggtatccgagcccgtgaccatcacctcgggccctgtggtggatccccccgccgtaataacgatgccacttgacggaccggccccaaacgggggatttcggcgtattccccggggggccctgcataccccggtcccgtcggaccaggctcgcaaggcgtactgtacccccgaaaccatcgcccgtctggtcgacgacccattgtttcccacggcctggcgccctgcgctaagctttgatcccggcgccttggcggaaatcgccgctcggcgtccgggcggaggagaccgacggtttggtccacccagcggagtggaggcgctgcgacggaggtgcgcctggatgcggcagatcccagacccggaggatgtgaggcttctgatcatctacgatccgttgcccggagaggacatcaacggccccctcgagagcaccctcgcgacagatccgggaccgtcatggagtccatcccgagggggactgtctgtggtcctggcagccctgagtaaccggttgtgcctgccgagcactcatgcctgggccgggaactggaccggcccgccggacgtgtccgctttgaacgcccggggcgttttattactgtcgacccgagacctggcctttgccggggccgtcgagtatctaggctcgcggttggcctctgcccggcgccggttgctggtgttggacgcggtggccctcgagaggtggcccggggatggacccgctttgtctcagtatcacgtgtacgtccgggccccggcgcgaccggacgcccaggccgtcgtccgatggccagactcggcggtcacagaaggactcgcccgggccgtgtttgcatcgtcgcgcacctttgggccagcgagttttgctcgtatcgagactgcgtttgccaacctgtacccgggcgaacaacccctgtgtttgtgccgcggtgggaacgtcgcatacaccgtgtgtacccgcgcgggccccaagacccgcgtccccctgtcgccccgtgaataccggcagtacgtgctgccgggttttgacggttgcaaggacctcgcgcgacagtctcggggtctggggctcggggcagccgactttgtggacgaggcggcacatagccaccgcgcagcaaaccgatggggcctgggtgccgcgcttcgacccgtcttccttcccgagggacggagaccgggggccgccgggccggaggccggcgacgtacccacctgggcgagggtgttttgccgccacgccctgctggaacccgaccctgccgcggaaccactcgtgcttccacccgtggccggtcggtcggtggcgctgtatgcgtcggcggacgaggctcggaatgccctccccccgattcccagagtaatgtggccgcccggttttggggccgcggagacggtgttggaggggagcgacggaacacggttcgcgttcggacaccacgggggctcggaacggccggcagaaacccaggcggggcgacagcggcgcaccgcagacgacagagaacacgctttggagccggacgattgggaggtggggtgtgaagacgcgtgggacagcgaggaggggggcggggacgacggggacgcaccggggtcatcctttggggtgagcgtcgtgtcggtggccccgggtgtgctgcgagaccgccgggtgggctcgcgcccggcggtcaaggtggagctgttgtcctcgtcctcgtcctccgaggacgaggacgatgtgtggggagggcgcggggggaggagccccccgcagagtcgggggtgacggagtcccctccttttctcgtgagcgccaccggcgcgcggactgtttgttgtttgttaataaaagcggaacggtttttatgaaaaaagtgtctgtctgtctgtgcgggcgggcgacgggcgggctggtcggaccccccccgaaaataaccccccccggtttctgggcgcccggcggaccccggggggggg

SEQ ID NO: 2 - DNA, 666 bp, Varicella-zoster virus
atggccaccgtgcactactcccgccgacctgggaccccgccggtcaccctcacgtcgtcccccggcatggatgacgttgcgacccccattccctacctacccacatacgccgaggccgtggcagacgcgccccccccttacagaagccgcgagagtctggtgttctccccgcctctttttcctcacgtggagaatggcaccacccaacagtcttacgattgcctagactgcgcttatgatggaatccacagacttcagctggcttttctaagaattcgcaaatgctgtgtaccggcttttttaattctttttggtattctcacccttactgctgtcgtggtcgccattgttgccgtttttcccgaggaacctcccaactcaactacacgaaactactgtccggaaggggaaggtatttattctcgcttgcagcttgtcgcgcgtgtatgcacaacaaaagctatatatgtcaccaaagccaacgtcgccatctggagtactacacccagtacattgcataacctgtccatctgcattttcagttgcgcggacgcctttctccgggatcgtggccttgggacatcaaccagcggaataagaaccgccggtggtcttgcccgaacgacgagtggcgacgcgttgttctgcataagctctgtatgctga

SEQ ID NO: 3 - DNA, 1365 bp, Varicella-zoster virus
atggggctctttggactgacacgctttatccatgaacataaactggttaaacccagcatcatttcaacgccacccggagttttaacccccgtggcggtagacgtatggaacgtcatgtacacattgttggaacgtttataccctgtgggtaaacgcgagaatttacacggaccatctgtaacgatacattgtcttggagtcttattgcggctattaacacaacggtcatactatccgatatttgtattggaacgttgtacagacggcccattatcacgtggagccaaggcaattatgtcacgggccatgaaccacgatgaaaggggaacctcggacttaacccgtgttctactatcatccaacacatgttctatcaagtataacaaaacatcggaaacatatgacagtgtgtttcgaaactcttccgcgagttgtattcctagcgaagaaaacaaatcccaggatatgtttttggacggttgtccacgacaaactgacaagatgatctgcctgcgcgaccaaaacgtatgcagtcttacctctacaatgccatcccgaggacatcctaaccatcgattatatcacaaattgtgtgcaagtcttattagatggatggggtatgcatacgtcgaggcggttgacattgaggcggacgaggcatgtgcaaacttatttcatacgcgtacagtggctttggtttatacgacagatactgatttactcttcatgggctgtgatattttgttagatgcaattcctatgtttgctccagtagtacgatgtcgcgatttgcttcaatatttaggaattacataccctgaatttttggttgcctttgttcgctgtcagaccgatttgcatacaagtgacaacctaaaatctgttcagcaagttattcaggataccggcctgaaaattccacatcaaatggacacttcaacgcgctcccccacttacgactcgtggagacatggcgaggttttcaaaagtcttaccgtagccacgtcgggtaaaacagaaaacggagtgtccgtttccaaatatgcatctaaccgatcggaggtgacagtagacgccagttgggctttaaaccttctgccaccctcatcctccccattggataatttggaacgcgcatttgttgaacatataatcgccgtggtaactccattgacccgcggtcgcctaaagttaatgaaacgtgtaaatattatgcaaaatacggcagacccatatatggttattaacaccttatatcataacttaaagggggaaaaaatggctcgccaatacgcacgtatttttaaacagtttattcctactccactcccactaaacactgtattaacaaaatattggaattaa

SEQ ID NO: 4 - DNA, 3600 bp, Varicella-zoster virus
atggaaaatactcagaagactgtgacagtgcccacggggcccctgggttacgtttatgcgtgccgggttgaagatttggatctggaggaaatttcatttttggccgctcgtagcacggactctgatttggctttattacctttgatgcgtaatttgaccgtggaaaaaacttttacatccagcctggcggtggtttctggagcacgcactacgggtcttgccggagctggtattaccttaaaactcactaccagtcatttctatccatctgtctttgtctttcacggaggcaaacacgttttacccagctctgcggccccaaatctcacacgcgcgtgtaacgcggctcgagaacggtttgggttttcacgctgccaagggcctcctgttgacggtgctgttgagacgaccggcgctgagatatgcacccgccttggattagagccagaaaatacaatattatacttggtggtcacggcattgtttaaggaagccgtatttatgtgcaacgtgtttctgcattatggaggactcgatattgttcatattaaccatggggatgttatacgtataccgttatttccggtacaacttttcatgcccgatgttaaccgtctggtacccgacccattcaacactcatcacaggtctatcggagagggttttgtatacccaacacccttttataacaccgggttgtgccatttaatacatgactgtgttattgctcccatggccgttgccttgcgcgtcagaaatgtaactgccgtcgcccgaggagcggcccaccttgcttttgatgaaaatcacgagggggcagtactcccccctgacattacgtacacgtattttcagtcctcttcaagtggaaccactaccgcccgtggagcgcgtcgaaacgatgtcaactccacgtctaagcctagcccatcgggggggtttgaaagacggttggcgtctattatggccgctgacacagccttgcacgcagaagttatattcaacactggaatttacgaagaaactccaacagatatcaaagaatggccaatgtttataggcatggagggcactttgccaaggctaaacgctctggggtcatataccgctcgtgtggccggggtcattggtgcgatggttttcagcccaaattctgcgttgtatctaactgaggtggaggatagcgggatgaccgaagccaaggatgggggaccgggtccatcatttaatcgattttaccagtttgccggacctcatttagctgcgaatccccaaacagatcgagatggccacgttctatccagtcagtctacgggttcatcaaacacagagtttagcgtggattatttggcactcatttgtggatttggagcacccctgttggcgcgactgcttttttatctagaacgctgtgacgctggtgcgtttacagggggtcacggggatgcgttaaaatatgttacggggacctttgactctgaaattccatgtagtttatgtgaaaaacacacgcggccggtatgcgctcacacaacagtacaccgacttagacaacgcatgccgcgatttggacaagccacccgtcaacctattggggtgtttggaacaatgaacagccaatatagcgactgcgatcctctaggaaactatgctccatatttaatccttcgaaaacccggggatcaaacggaagcagcaaaggcaaccatgcaggacacttatagggctacactagaacgcttgtttatcgatctagaacaagagcgactactggatcgcggtgccccatgttcttccgagggactatcgtctgtcattgtggatcatccaacgtttcgtcgcatattagacacactgcgtgcgcgtatagaacagacaacaacacaatttatgaaagtgttggttgagacccgcgattataagatccgtgaaggattatccgaagccacccattcaatggcgttaacgtttgatccatactcaggagcattttgtcccattaccaattttttagttaaacgaacacacctagccgtggtacaagacttagcattaagccagtgtcattgtgtattttacggacagcaagttgaggggcggaactttcgtaaccaattccaacctgttttgcggcggcgttttgttgacctgtttaatggggggtttatatcaacacgctctataaccgtaacattatctgaaggtcctgtatccgccccaaatccgacattgggacaagacgcgcccgcggggcgtacctttgatggggatttagcgcgcgtaagcgtggaagttattcgggatatacgagttaaaaatagggtcgttttttcaggtaactgtacaaatctctctgaggcagcccgggcaaggcttgtaggccttgcaagtgcgtaccaacgccaagaaaaaagagtggatatgttacacggggccctagggtttttgcttaaacagtttcacggcctgttatttcctcggggtatgccaccaaacagtaaatcccccaacccgcagtggttttggaccctgttacaacgcaaccagatgccggcagataaacttacacacgaagagattaccactattgcagctgttaaacggtttaccgaggaatatgcagcactaaactttattaatctacccccaacctgcataggagaattagcccagttttatatggcaaatcttattcttaaatactgcgatcattcacagtaccttataaataccttaacttctataattacgggtgccaggcgcccgcgtgacccatcatccgttttgcattggattcgtaaagatgtcacgtccgccgcggacatagaaacccaagcaaaggcgcttcttgaaaaaacggaaaacttaccggaattatggactacggcttttacttcaactcatttagtccgcgcggccatgaatcaacgtcccatggtcgttttaggaataagcattagtaaatatcacggagcggcaggaaacaaccgcgtctttcaggcagggaattggagcggtttaaacgggggtaaaaatgtatgcccgctatttacatttgatcgcactcgccgttttataataacatgtcctagaggaggttttatctgccccgtaacaggtccctcgtcgggaaatcgagaaaccaccctatccgaccaagttcgcggtataattgtcagtggcggggccatggttcaattagccatatacgccacggttgtgcgtgcagtgggcgctcgagcacaacatatggcatttgacgactggttaagtcttacagacgatgagtttttagccagagacttggaggagttacacgaccagattatccaaaccctggaaacgccctggaccgtagaaggcgctctagaagcagtaaagattctagatgaaaaaacgacagcgggagatggggaaacccccacaaacctagcatttaattttgattcttgtgaaccaagccatgacaccacatctaacgtattaaacatttcagggtcaactgtccctggtcttaaacgaccccccgaagatgacgaactctttgatcttagtggtattcccataaaacatgggaacattacaatggaaatgatttaa

SEQ ID NO: 5 - DNA, 732 bp, Varicella-zoster virus
atgaaaaatccgcagaaattagcgatcacattcttgccgctctacgtgatcccaacgtacacgttgtgtattaaagcattgtataaaaacacgcatgcgggcttgctgttctcatttctaggttttgtcttaaatacacccgccatgagcatctctggacccccaacgacgtttattttatataggttacatggggtcaggcgggttcttcactggactttaccggatcatgaacaaaccctctacgcatttacgggtgggtcaagatcaatggcggtgaagacggacgctcgatgtgatacaatgagcggtggtatgatcgtccttcaacacacccatacagtgaccctgctaaccatagactgttctactgacttttcatcatacgcatttacgcaccgggatttccacttacaggacaaaccccacgcaacatttgcgatgccgtttatgtcctgggtcggttctgacccaacatctcagctgtacagtaatgtggggggggtactatccgtaataacggaagatgacctatccatgtgtatctcaattgttatatacggtttacgggtaaacagacctgacgatcagaccacaccaacaccaaccccgcaccagtatacatcgcaaaggcggcagcctgaaaccaactgtccttcaccacaaccggcctttttcacatcagacgacgacgttctttcgttaatattacgggacgccgcaaacgcgtaa

SEQ ID NO: 6 - DNA, 483 bp, Varicella-zoster virus
tcattggcatacgcgttggaacaaacggtcggctccccagacacatccattttccgggatatttgtggaagatggagtagagtctacccatacaccggaaagggcatccaacaaagcatcgcgtatgtccccgcttttatgttcttcaccaacagattgtgtcagcccctttaaggtgacgtatggatttgtccagtacgccatttgtttgtctttaaaccaaagtataacttccggtactggacattttgtcttaaccacgattcccgatagcgcctcgctgaggtttgataccgggggtgccgcatagtcccacgcctcatataccgatgacacgcacggttccgttataatcaaactcacatccgatagcggtttggctccaaaaaacaacggagtgtcgtcttggagatgaagacaatacgcgattgtgatagtttttaaaaaaactatcatctgcagtaaccatttatgtgatgccat

SEQ ID NO: 7 - DNA, 18 bp, Artificial Sequence
  Description of Artificial Sequence Synthetic consensus oligonucleotide
ggacgcgatcgacgacga

SEQ ID NO: 8 - DNA, 15 bp, Artificial Sequence
  Description of Artificial Sequence Synthetic consensus oligonucleotide
gggagaggcggagga

SEQ ID NO: 9 - DNA, 43 bp, Artificial Sequence
  Description of Artificial Sequence Synthetic consensus oligonucleotide
acctcggccgctwcccgaaanncccgatcccgccgtcgcgccc

SEQ ID NO: 10 - DNA, 27 bp, Artificial Sequence
  Description of Artificial Sequence Synthetic consensus oligonucleotide
ccccgccgatggggagggggcgcggta

SEQ ID NO: 11 - DNA, 15 bp, Varicella-zoster virus
aacatttcagggtca

SEQ ID NO: 12 - DNA, 11 bp, Varicella-zoster virus
caactacatga

SEQ ID NO: 13 - DNA, 21 bp, Varicella-zoster virus
caactacacgaaactactgtc

SEQ ID NO: 14 - DNA, 22 bp, Varicella-zoster virus
ctgcataagctctgtatgctga

SEQ ID NO: 15 - DNA, 22 bp, Varicella-zoster virus
atccaacacatcatgttctatc

SEQ ID NO: 16 - DNA, 19 bp, Varicella-zoster virus
atccaacacatgttctatc

SEQ ID NO: 17 - protein, 11 aa, Varicella-zoster virus
LSSNTSCSIKY

SEQ ID NO: 18 - protein, 10 aa, Varicella-zoster virus
LSSNTCSIKY

SEQ ID NO: 19 - DNA, 20 bp, Varicella-zoster virus
aactgtccttcttcaccaca

SEQ ID NO: 20 - DNA, 17 bp, Varicella-zoster virus
aactgtccttcaccaca

SEQ ID NO: 21 - protein, 11 aa, Varicella-zoster virus
ETNCPSSPQPA

SEQ ID NO: 22 - protein, 10 aa, Varicella-zoster virus
ETNCPSPQPA

SEQ ID NO: 23 - DNA, 36 bp, Varicella-zoster virus
aacatttcagggtcaaacatttcagggtcaactgtc

SEQ ID NO: 24 - DNA, 21 bp, Varicella-zoster virus
aacatttcagggtcaactgtc

SEQ ID NO: 25 - protein, 16 aa, Varicella-zoster virus
LNISGSNISGSTVPGL

SEQ ID NO: 26 - protein, 11 aa, Varicella-zoster virus
LNISGSTVPGL

SEQ ID NO: 27 - DNA, 18 bp, Varicella-zoster virus
ttactgcagatagttttt

SEQ ID NO: 28 - DNA, 21 bp, Varicella-zoster virus
ttactgcagatgatagttttt

SEQ ID NO: 29 - DNA, 18 bp, Varicella-zoster virus
ttactacagatagttttt

SEQ ID NO: 30 - protein, 11 aa, Varicella-zoster virus
HKWLLQIVFLK

SEQ ID NO: 31 - protein, 12 aa, Varicella-zoster virus
HKWLLQMIVFLK

(19) United States
(12) Patent Application Publication
(10) Pub. No.: US 2014/0147458 A1
(43) Pub. Date: May 29, 2014

(54) NOVEL VARICELLA-ZOSTER VIRUS STRAINS, AND CHICKEN POX AND HERPES ZOSTER VIRUS VACCINE USING SAME

(71) Applicants: Geun Hee Kim, Yongin-si (KR); Shi Nae Kwon, Yongin-si (KR); Chan Hee Lee, Cheongju-si (KR); Yeop Yoon, Yongin-si (KR)

(72) Inventors: Geun Hee Kim, Yongin-si (KR); Shi Nae Kwon, Yongin-si (KR); Chan Hee Lee, Cheongju-si (KR); Yeop Yoon, Yongin-si (KR)

(73) Assignee: MOGAM BIOTECHNOLOGY RESEARCH INSTITUTE, Yongin-si, Kyunggi-do (KR)

(21) Appl. No.: 14/000,770

(22) PCT Filed: Feb. 24, 2012
(86) PCT No.: PCT/KR2012/001408

§ 371 Date: Aug. 21, 2013

Related U.S. Application Data
(60) Provisional application No. 61/446,284, filed on Feb. 24, 2011.

(51) Int. Cl. C07K 14/005

(52) U.S. Cl. CPC C07K 14/005
USPC 424/186.1; 536/23.72; 530/350; 435/320.1; 435/325; 435/419; 435/254.2; 435/252.33; 435/348; 435/365; 435/362; 435/352; 435/350; 435/369; 435/252.31; 435/252.35; 435/252.34; 435/254.3; 435/254.23; 435/254.21

(57) ABSTRACT

The present invention relates to novel Varicella-zoster virus strain and to a chicken pox and herpes zoster virus vaccine using the same. More particularly, the present invention relates to genome DNA of VZV MAV/06 strain isolated from a Korean patient, to an open reading frame (ORF) thereof, to a protein coded by the genome DNA of VZV MAV/06 strain and the ORF thereof, and to a vaccine composition which contains the protein as an active ingredient.

### CROSS REFERENCE TO RELATED APPLICATIONS

This is a National Stage of International Application No. PCT/KR2012/001408, filed Feb. 24, 2012, claiming priority from U.S. Patent Provisional Application No. 61/446,284, filed Feb. 24, 2011, the contents of all of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Aug. 19, 2013, is named Q206364 SL.txt and is 176,706 bytes in size.

### TECHNICAL FIELD

The present invention relates to novel varicella-zoster virus (VZV) strains and a chicken pox and herpes zoster virus vaccine using the same. More particularly, the present invention relates to genome DNA of VZV MAV/06 strain isolated from a Korean patient and attenuated, an opening reading frame (hereinafter, referred to as ORF) thereof, a protein coded by the genome DNA of VZV MAV/06 strain and the ORF thereof, and a vaccine composition containing the protein as an active ingredient.

### BACKGROUND ART

Varicella-zoster virus (hereinafter, referred to as VZV) belongs to the Herpes viridae family and is a cause of a disease with two different symptoms (chicken pox and herpes zoster).

As a VZV strain isolated from clinical specimens, a perfect nucleotide sequence of a Dumas strain has been initially reported (Davison, A. J. and Scott, J. E., 1986). In addition, the completed entire nucleotide sequences of 23 VZV strains including a parental Oka strain and three vaccine virus strains derived from the parental Oka strain (vOka strain, Varivax strain, and Varilrix strain) have been reported to NCBI GenBank database up to now.

Currently, research into a specific region for attenuating vaccine strain may be conducted through comparative analysis of the entire nucleotide sequences in the vaccine virus strain and clinical virus strain (Argaw et al., 2000; Gomi et al., 2002; Tillieux et al., 2008; Yamanish, 2008).

Meanwhile, “Suduvax” has been mainly produced by Green Cross Corp. as an attenuated vaccine against chicken pox since 1994. A MAV/06 virus strain used to produce Suduvax was obtained by isolating Varicella-zoster virus from blisters of a 33 month old Korean patient infected by chicken pox in 1989 and primarily culturing the isolated VZV in human embryonic lung cells, and it was confirmed by testing various characteristics of varicella-zoster virus that the cultured VZV was a novel VZV (Park et al., Propagation of Varicella-Zoster Virus isolated in Korea, J. Kor. Soc. Virol. 21, 1-9, 1991).

The Suduvax has been commercialized in the Korean market since 1994 and used in the global market since 1998. However, even though efficacy and safety of the Suduvax have been proven in the market, molecular biological characteristics capable of describing a mechanism of efficacy and attenuation of the vaccine have not yet been reported.

The molecular biological characteristics are significantly important to increase accuracy of quality control and quality assurance for assuring efficacy, safety, and homogeneity of the attenuated vaccine and should be urgently investigated.

### DISCLOSURE

### Technical Problem

In order to solve this problem, the present inventors found out a genome sequence of a novel VZV MAV/06 strain isolated from a Korean patient and performed molecular biological analysis including ORF analysis and phylogenetic analysis the isolated VZV MAV/06 strain, thereby completing the present invention.

An object of the present invention is to provide a genome DNA of VZV MAV/06 strain.

Another object of the present invention is to provide an open reading frame (ORF) of the genome DNA of VZV MAV/06 strain.

Still another object of the present invention is to provide a protein coded by the ORF of the genome DNA of VZV MAV/06 strain.

Still another object of the present invention is to provide a recombinant expression vector including the genome DNA of VZV MAV/06 strain or the ORF thereof.

Still another object of the present invention is to provide a transformant including the genome DNA of VZV MAV/06 strain or the ORF thereof.

Still another object of the present invention is to provide a method of preparing protein coded by the genome DNA of VZV MAV/06 strain or the ORF thereof.

Still another object of the present invention is to provide a vaccine composition containing the protein of VZV MAV/06 strain as an active ingredient.

### Technical Solution

In order to achieve the objects, the present invention provides genome DNA of VZV MAV/06 strain and an ORF thereof.

Hereinafter, the present invention will be described in detail.

As used herein, the term ‘open reading frame’ or ‘ORF’, which is a DNA base sequence means a DNA base sequence translated into an amino acid sequence, means a base sequence from a translation initiation codon (for example, ATG) to a termination codon (for example TGA, TAA, and TAG).

In one general aspect, the present invention provides genome DNA of Varicella-zoster virus (VZV) MAV/06 strain having a base sequence of SEQ ID NO. 1.

In another general aspect, the present invention provides an ORF of the genome DNA of VZV MAV/06 strain. In more detail, the ORF may be selected from an ORF 0 having a base sequence of SEQ ID NO. 2, an ORF 17 having a base sequence of SEQ ID NO. 3, an ORF 29 having a base sequence of SEQ ID NO. 4, an ORF 56 having a base sequence of SEQ ID NO. 5, and an ORF 60 having a base sequence of SEQ ID NO. 6.

In another general aspect, the present invention provides a protein coded by the genome DNA of VZV MAV/06 strain or the ORF thereof. In more detail, the present invention provides a protein coded by genome DNA of VZV MAV/06 strain having a base sequence of SEQ ID NO. 1 or an ORF selected from an ORF 0 having a base sequence of SEQ ID NO. 2, an ORF 17 having a base sequence of SEQ ID NO. 3, an ORF 29 having a base sequence of SEQ ID NO. 4, an ORF 56 having a base sequence of SEQ ID NO. 5, and an ORF 60 having a base sequence of SEQ ID NO. 6.

In another general aspect, the present invention provides a recombinant expression vector including the genome DNA of VZV MAV/06 strain or the ORF thereof. In more detail, the present invention provides a recombinant expression vector including genome DNA of VZV MAV/06 strain having a base sequence of SEQ ID NO. 1 or an ORF selected from an ORF 0 having a base sequence of SEQ ID NO. 2, an ORF 17 having a base sequence of SEQ ID NO. 3, an ORF 29 having a base sequence of SEQ ID NO. 4, an ORF 56 having a base sequence of SEQ ID NO. 5, and an ORF 60 having a base sequence of SEQ ID NO. 6.

As used herein, the term “recombinant vector”, which is an expression vector capable of expressing a target protein in a suitable host cell, means a genetic construct containing an essential regulatory element to which a genetic insert is operably linked so as to be expressed. As used herein, the term “operably linked” means that a nucleic acid expression regulatory sequence and a nucleic acid sequence coding the target protein are functionally linked to each other so as to perform general functions. The operable linkage with the recombinant vector may be performed using a genetic recombinant technology well known in the art to which the present invention pertains, and a region-specific DNA cleavage and linkage may be easily performed using an enzyme generally known in the art to which the present invention pertains.

A suitable expression vector capable of being used in the present invention may include a signal sequence for membrane targeting or secretion in addition to expression regulatory elements such as a promoter, an initiation codon, a termination codon, a polyadenylation signal, and an enhancer. The initiation codon and the termination codon may be generally considered as a portion of a nucleotide sequence coding an immunogenic target protein, necessary to be functional in an individual to whom a genetic construct has been administered, and must be in a frame together with the coding sequence. The promoter may be generally constitutive or inducible. An example of the promoter available in prokaryotic cells may include lac, tac, T3, and T7 promoters, but is not limited thereto. An example of the promoter available in eukaryotic cells may include β-actin promoter, human hemoglobin promoter, human muscle creatine promoter, and human metallothionein promoter, as well as simian virus 40 (SV40) promoter, mouse mammary tumor virus (MMTV) promoter, human immunodeficiency virus (HIV) promoter such as the HIV Long Terminal Repeat (LTR) promoter, Moloney virus promoter, cytomegalovirus (CMV) promoter, Epstein Barr virus (EBV) promoter, and Rous sarcoma virus (RSV) promoter, but is not limited thereto. The expression vector may include a selectable marker for selecting host cells containing the vector. The selectable marker is to select cells transformed by the vector, and as the selectable marker, markers conferring selectable phenotypes such as drug resistance, nutrient requirement, cytotoxic agent resistance, or expression of surface proteins may be used. Since only cells expressing the selectable marker survive in the environment treated with a selective agent, transformed cells may be selected. Further, in the case of a replicable expression vector may include a replication origin, which is a specific nucleic acid sequence that initiates replication. As the recombinant expression vector, a vector having various shapes such as plasmid, virus, cosmid, and the like, may be used. A kind of recombinant vector is not particularly limited as long as the vector may serve to express the desired gene in various host cells such as prokaryotic cells and eukaryotic cells and produce the desired protein, but a vector capable of producing a foreign protein having a form similar to that in a natural state on a large scale while having a promoter having a strong activity and strong expression capacity may be preferable.

In order to express the protein of VZV MAV/06 strain coded by any one selected from base sequences of SEQ ID NOS. 1 to 6 according to the present invention, various expression host/vector combinations may be used. As an expression vector suitable for eukaryotic cells, an expression regulatory sequence derived from SV40, bovine papilloma virus, adenovirus, adeno-associated virus, cytomegalovirus, and retrovirus, and the like, may be used, but the present invention is not limited thereto. An expression vector usable in bacterial host cells may include a bacterial plasmid obtained from Escherichia coli such as pET, pRSET, pBluescript, pGEX2T, pUC vector, col E1, pCR1, pBR322, pMB9, and a derivative thereof, plasmids having a broader host range such as RP4, phage DNA exemplified as various phage lambda derivatives such as λgt10, λgt11, NM989, and other DNA phages such as M13, single-stranded filament type DNA phage, or the like. An expression vector available in yeast cells may be 2° C. plasmid and a derivative thereof. A vector useful for insect cells may be pVL941.

The recombinant vector may be inserted in a host cell to form a transformant. In this case, a suitable host cells may be prokaryote cells such as Escherichia coli Bacillus subtilis, Streptomyces sp., Pseudomonas sp., Proteus mirabilis, or Staphylococcus sp. In addition, the suitable host cells may be fungi such as Aspergillus sp., yeasts such as Pichia pastoris, Saccharomyces cerevisiae, Schizosaccharomyces sp., and Neurospora crassa, eukaryotic cells such as lower eukaryotic cells, higher eukaryotic cells, for example, insect cells, or the like.

The host cell may be preferably derived from plants and/or mammals. In particular, monkey kidney cells 7 (COS7), NSO cells, SP2/O, Chinese hamster ovary (CHO) cells, W138, baby hamster kidney (BHK) cells, Madin-Darby canine kidney (MDCK) cells, myeloma cell lines, HuT 78 cells, HEK293 cells, and the like, may be used, but the present invention is not limited thereto. Particularly, the CHO cells may be preferable.

As used herein, the term “transformation into host cells” may include any method for introducing nucleic acids into organisms, cells, tissues, or organs and be performed by selecting standard techniques suitable for the host cell as known in the art. These methods may include an electroporation method, a protoplast fusion method, a calcium phosphate (CaPO₄) precipitation method, a calcium chloride (CaCl₂) precipitation method, an agitation method using silicon carbide fiber, an agrobacterium-mediated transformation method, and PEG-, dextran sulfate-, or lipofectamine- and desiccation/inhibition-mediated transformation, but are not limited thereto.

The transformed host cells in which the recombinant vector is expressed are cultured in a nutrient medium, such that the protein of VZV MAV/06 strain coded by any one selected from base sequences of SEQ ID NOS. 1 to 6 according to the present invention may be produced on a large scale. Here, general medium and culturing conditions may be suitably selected according to the host cell. Conditions such as a temperature, pH of the medium, a culturing time, and the like, may be controlled appropriately for cell growth at the time of culturing the cell and mass production of the protein. The protein of VZV MAV/06 strain coded by any one selected from base sequences of SEQ ID NOS. 1 to 6 recombinantly produced as described above may be recovered from lysates of the medium or cells and be separated and purified by a general biochemical separation technology (Sambrook et al., Molecular Cloning: A Laboratory Manual, 2nd Ed., Cold Spring Harbor Laboratory Press (1989); Deuscher, M., Guide to Protein Purification Methods Enzymology, Vol. 182. Academic Press, Inc., San Diego, Calif. (1990)). As the separation technology, electrophoresis, centrifugation, gel filtration, precipitation, dialysis, chromatography (ion exchange chromatography, affinity chromatography, immuno-adsorption chromatography, size exclusion chromatography), isoelectric focusing, and various modification and composite methods thereof, may be used, but the present invention is not limited thereto.

In another general aspect, the present invention provides a vaccine composition containing the protein of VZV MAV/06 strain coded by any one selected from base sequences of SEQ ID NOS. 1 to 6 as an active ingredient. In this case, the vaccine composition may further contain a pharmaceutically acceptable adjuvant or excipient. Any adjuvant may be used as long as it serves to promote formation of antibody at the time of injecting the adjuvant into the body to thereby achieve the objects of the present invention. Particularly, at least one selected from an aluminum salt (Al(OH)₃ or ALPO₄), squalene, sorbitane, polysorbate 80, CpG, liposome, cholesterol, monophosphoryl lipid (MPL) A, and glucopyranosyl lipid A (GLA) may be preferably used, but the present invention is not limited thereto.

### Advantageous Effects

According to the present invention, the entire genome sequence of Varicella-zoster virus (VZV) MAV/06 strain isolated from a Korean patient was analyzed and compared with base sequences of 23 VZV virus strains, such that polymorphism of a length of the genome between species due to deformation of reiteration sequences and an origin of replication may be confirmed. In addition, the entire sequence of VZV MAV/06 strain virus may assist in understanding molecular characteristics of attenuated VZV vaccine virus strains through bioinformatic research, comparative genomic research, and the like.

### DESCRIPTION OF DRAWINGS

FIG. 1 shows an ORF map of VZV MAV/06 strain (hereinafter, represented by ‘Suduvax’ in the drawings) according to the present invention;

FIG. 2 shows a phylogenetic tree of 24 VZV virus strains based on the entire nucleotide sequences;

FIG. 3 shows a phylogenetic tree of the 24 VZV virus strains based on non-coding nucleotide sequences;

FIG. 4 shows a characteristic phylogenetic tree of vaccine virus strains based on 12 ORF differences between the vaccine virus strains and clinical virus strains;

FIG. 5 shows correlation between the lengths of the genomes and reiteration sequences of the VZV virus strains;

FIG. 6 shows mutants between an ORFO of the VZV MAV/06 strain according to the present invention and Oka vaccine virus strains. FIG. 6 discloses “CAACTACATGA” as SEQ ID NO: 12, “CAACTACACGAAACTACTGTC” as SEQ ID NO: 13, and “CTGCATAAGCTCTGTATGCTGA” as SEQ ID NO: 14;

FIG. 7 shows a 3-bp deletion region in an ORF17 of the VZV MAV/06 strain according to the present invention. FIG. 7 discloses “ATCCAACACATCATGTTCTATC” as SEQ ID NO: 15, “ATCCAACACATGTTCTATC” as SEQ ID NO: 16, “LSSNTSCSIKY” as SEQ ID NO: 17, and “LSSNTCSIKY” as SEQ ID NO: 18;

FIG. 8 shows a 3-bp deletion region in an ORF56 of the VZV MAV/06 strain according to the present invention. FIG. 8 discloses “AACTGTCCTTCTTCACCACA” as SEQ ID NO: 19, “AACTGTCCTTCACCACA” as SEQ ID NO: 20, “ETNCPSSPQPA” as SEQ ID NO: 21, and “ETNCPSPQPA” as SEQ ID NO: 22;

FIG. 9 shows a 15-bp deletion region in an ORF29 of the VZV MAV/06 strain according to the present invention. FIG. 9 discloses “AACATTTCAGGGTCAAACATTTCAGGGTCAACTGTC” as SEQ ID NO: 23, “AACATTTCAGGGTCAACTGTC” as SEQ ID NO: 24, “LNISGSNISGSTVPGL” as SEQ ID NO: 25, and “LNISGSTVPGL” as SEQ ID NO: 26; and

FIG. 10 shows a 3-bp insertion region in an ORF60 of the VZV MAV/06 strain according to the present invention. FIG. 10 discloses “TTACTGCAGATAGTTTTT” as SEQ ID NO: 27, “TTACTGCAGATGATAGTTTTT” as SEQ ID NO: 28, “TTACTACAGATAGTTTTT” as SEQ ID NO: 29, “HKWLLQIVFLK” as SEQ ID NO: 30, and “HKWLLQMIVFLK” as SEQ ID NO: 31.

### BEST MODE

Hereinafter, the present invention will be described in detail with reference to Examples and the accompanying drawings. However, the Examples and drawings of the present invention have been disclosed for illustrative purposes, but the scopes of the present invention are not limited thereby.

### Example 1

### Virus and DNA Sequencing

A MAV/06 virus strain was obtained by isolating Varicella-zoster virus from blisters of a 33 month old Korean patient infected by chicken pox in 1989 and primarily culturing the isolated VZV in human embryonic lung cells (Hwang, K. K., Park, S. Y., Kim, S. J., Ryu, Y. W., & Kim, K. H., Restriction Fragment Length Polymorphism Analysis of Varicella-Zoster Virus isolated in Korea. J. Kor. Soc. Virol. 21, 201-210, 1991).

The cultured MAV/06 virus strain was sub-cultured 55 times in human and guinea pig embryonic diploid lung cells at 34 to 36° C. and it was confirmed through cell sensitivity and temperature sensitivity tests that the sub-cultured MAV/06 virus strain was attenuated (Hwang et al., Marker test for attenuation of varicella-zoster viruses isolated in Korea). The attenuated virus strain was named as ‘MAV/06’ and used to produce Suduvax.

The attenuated MAV/06 virus strain was serially sub-cultured in human embryonic diploid lung cells to prepare a master virus seed, and the prepared master virus seed was serially sub-cultured to prepare a working virus seed.

In addition, “Suduvax”, which is a final vaccine, was produced using a working virus seed sub-cultured 5 times, and sequence analysis was performed using the final vaccine.

DNA was extracted from the MAV/06 virus strain stock at a concentration of 5.5 μg/100 μl using a QIAamp DNA mini kit (Macrogen). A DNA sequence was determined by a Genome Sequencer FLX standard system of Roche Diagnostics.

23,722 sequence fragments with an average length of ˜400 bp were obtained and the obtained sequence fragments were assembled and viewed using a Consed program (http://bozeman.mbt.washington.edu/consed/consed.html). An average quality of the sequence fragments was 99.99%. A total of 99.38% of 124,759 sequences was matched with the derived consensus sequence and the coverage was 83 reads per nucleotide. These were aligned against two reference virus strains Dumas (NC_{—}001348) and Varilrix (DQ008354) and gaps between the contigs were filled by PCR Sequencing using primers obtained from the adjacent contigs.

The completed entire genome sequence of the MAV/06 virus strain was registered in NCBI Genbank database (Aug. 9, 2011) and represented by SEQ. ID NO. 1. As shown in SEQ. ID NO. 1, it was confirmed that the genome of the MAV/06 virus strain has a length of 124,759 bp.

### Example 2

### Analysis of Open Reading Frames

A location of open reading frames (ORFs) of the MAV/06 virus strain in the entire genome sequence was determined by Blast search for two reference virus strains Dumas (NC_{—}001348) and Varilrix (DQ008354). It may be confirmed that the resulting data included the first and last nucleotide positions of each ORF in the MAV/06 virus strain genome and direction of the ORFs.

The ORF information was verified by ORF finding programs such as CLC Sequence Viewer (version 6.1 http://www.cicbio.com/index.php) and ORF Finder provided by NCBI. When the results of the blast search did not coincide with those of ORF finding programs, the nucleotide sequences of the corresponding ORFs were examined with BioEdit Sequence Alignment Editor (Department of Microbiology, North Carolina State University, version 5.0.9 http://www/mbio.ncsu.edu/BioEdit/bioedit.html) and manually edited, thereby determining positions of initiation and termination codons. Finally, all the allocated ORFs were confirmed by identification of the translated amino acid sequences.

Results of the above-mentioned analysis are shown in the following items (1) to (4).

(1) MAV/06 Virus Strain Genome Structure and ORF Mapping

The structure of the MAV/06 virus strain genome was a typical structure of the VZV virus strain in that the genome may be divided into 6 regions, that is, TRL, UL, IRL, IRS, US, and TRS, and lengths of the regions were 88 bp, 104,799 bp, 88 bp, 7,267 bp, 5,232 b, and 7,276 bp, respectively.

A G+C content of the MAV/06 virus strain genome was approximately 46.1%, and the total genome length was significantly similar to those of 24 VZV virus strains. VZV virus strains analyzed in the present invention, and GenBank accession numbers, genome lengths, and G+C contents thereof were shown in the following Table 1.

TABLE 1
INFORMATION ON VZV VIRUS STRAINS ANALYZED IN THE PRESENT INVENTION

| | Accession | | Length (bp) | | | | | | | |
|---|---|---|---|---|---|---|---|---|---|---|
| Strain | Number | Country | Genome | TRl | Ul | IRl | IRs | Us | TRs | % G + C |
| Dumas | NC001348 | Netherlands | 124,884 | 88 | 104,836 | 88 | 7,320 | 5,232 | 7,320 | 46.0 |
| M2DR | DQ452050 | Morocco | 124,770 | 89 | 104,719 | 89 | 7,320 | 5,232 | 7,321 | 46.0 |
| CA123 | DQ457052 | USA | 124,771 | 100 | 104,698 | 98 | 7,322 | 5,232 | 7,321 | 46.0 |
| SD | DQ479953 | USA | 125,087 | 88 | 104,787 | 88 | 7,446 | 5,232 | 7,446 | 46.1 |
| Kel | DQ479954 | USA | 125,374 | 88 | 104,857 | 88 | 7,555 | 5,232 | 7,554 | 46.2 |
| 11 | DQ479955 | Canada | 125,370 | 88 | 104,906 | 88 | 7,529 | 5,232 | 7,527 | 46.2 |
| 22 | DQ479956 | Canada | 124,868 | 88 | 104,689 | 88 | 7,386 | 5,232 | 7,385 | 46.0 |
| 03-500 | DQ479957 | Canada | 125,239 | 88 | 105,299 | 88 | 7,266 | 5,232 | 7,266 | 46.1 |
| 36 | DQ479958 | Canada | 125,030 | 88 | 104,850 | 88 | 7,387 | 5,232 | 7,385 | 46.1 |
| 49 | DQ479959 | Canada | 125,041 | 88 | 104,916 | 88 | 7,358 | 5,232 | 7,359 | 46.1 |
| 8 | DQ479960 | Canada | 125,451 | 89 | 105,020 | 88 | 7,510 | 5,232 | 7,512 | 46.2 |
| 32p5 | DQ479961 | USA | 124,945 | 88 | 104,760 | 88 | 7,389 | 5,232 | 7,388 | 46.1 |
| 32p22 | DQ479962 | USA | 125,084 | 88 | 104,791 | 88 | 7,443 | 5,232 | 7,442 | 46.1 |
| 32p72 | DQ479963 | USA | 125,169 | 88 | 104,870 | 88 | 7,446 | 5,232 | 7,445 | 46.1 |
| NH29_3 | DQ674250 | USA | 124,811 | 87 | 104,766 | 87 | 7,320 | 5,230 | 7,321 | 46.0 |
| SVETA | EU154348 | Russia | 124,813 | 87 | 104,772 | 87 | 7,319 | 5,230 | 7,318 | 46.0 |
| MSP | AY548170 | USA | 124,883 | 88 | 104,848 | 88 | 7,313 | 5,232 | 7,314 | 46.0 |
| BC | AY548171 | Canada | 125,459 | 88 | 105,326 | 88 | 7,363 | 5,231 | 7,363 | 46.2 |
| HJO | AJ871403 | Germany | 124,928 | 89 | 104,752 | 89 | 7,335 | 5,230 | 7,433 | 46.0 |
| pOka | AB097933 | Japan | 125,125 | 88 | 104,798 | 88 | 7,463 | 5,225 | 7,463 | 46.1 |
| vOka | AB097932 | Japan | 125,078 | 88 | 104,822 | 88 | 7,427 | 5,232 | 7,421 | 46.1 |
| VarilRix | DQ008354 | Japan | 124,821 | 88 | 104,761 | 88 | 7,326 | 5,231 | 7,327 | 46.1 |
| VariVax | DQ008355 | Japan | 124,815 | 88 | 104,758 | 88 | 7,324 | 5,232 | 7,325 | 46.1 |
| MAV06 | This study | Korea | 124,759 | 88 | 104,799 | 88 | 7,276 | 5,232 | 7,276 | 46.1 |

The MAV/06 virus strain included 74 ORFs. It was confirmed that among them, 64 ORFs were UL genes, and 4 ORFs were US genes.

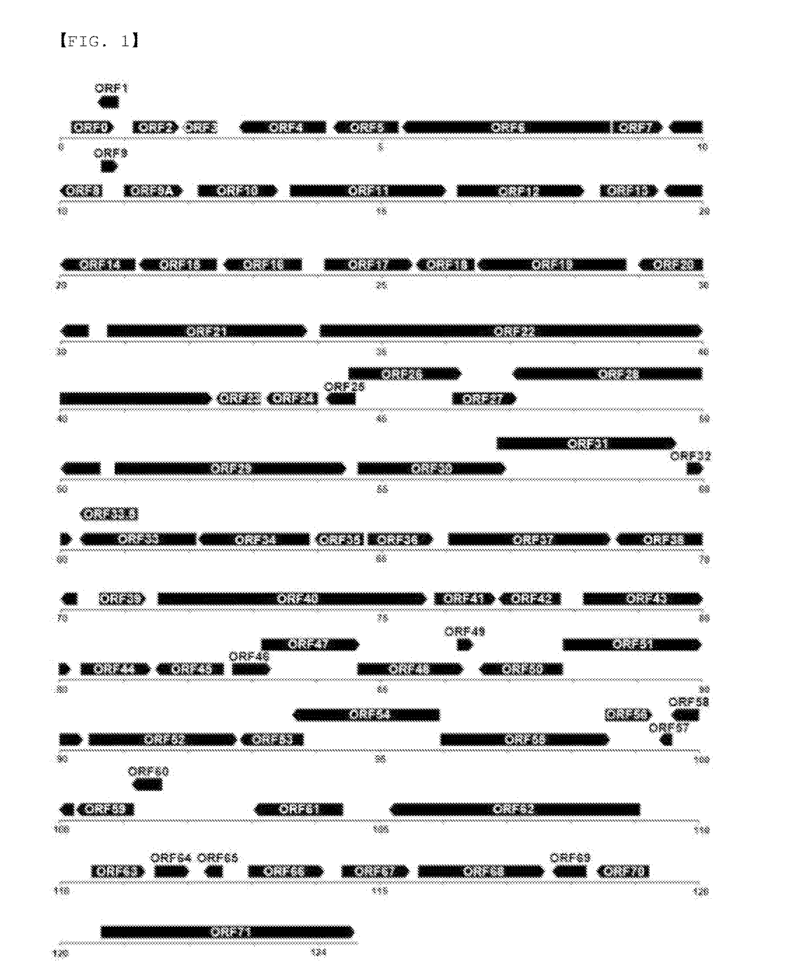

3 genes (ORFs 62-64) in IRS were repeatedly present in a reverse direction in the TRS(ORFs 69-71), and among 74 ORFs, 70 ORFs were present in a forward direction, ORFs were present in the reverse direction. In addition, the directions of ORFs were 100% conserved among the VZV strains. The ORF map of the MAV/06 virus strain (represented by ‘Suduvax’ in all of the drawings) was shown in FIG. 1.

(2) Phylogenetic Analysis of MAV/06 Virus Strain

Based on the entire nucleotide sequences of the virus strains of Table 1, phylogenetic trees were constructed using a neighbor-joining method.

The ORF nucleotide sequence of the MAV/06 virus strain was multiple-aligned with the nucleotide sequences and amino acids of 24 VZV strains of Table 1 registered in NCBI GenBank database, by way of ClustalW (ver. 2.0.1), followed by manual editing.

The resulting output files were used in order to construct the phylogenetic trees using Dnadist and neighbor programs included in Phylip package (version 3.69, http://evolution.genetics.washington.edu/phylip.html).Dista nce matrix was obtained by Kimura-2-parameter. Cluster analysis was performed by a neighbor-joining (NJ) method and a maximum-likelihood (ML) method, and the resulting tree files were viewed by the Treeview program (version 1.6.6). Significance of the phylogenetic trees was verified by bootstrap analysis. Phylogenetic trees were constructed from 1000 replicates generated by the Segboot program and the consensus tree was identified by the Consense program.

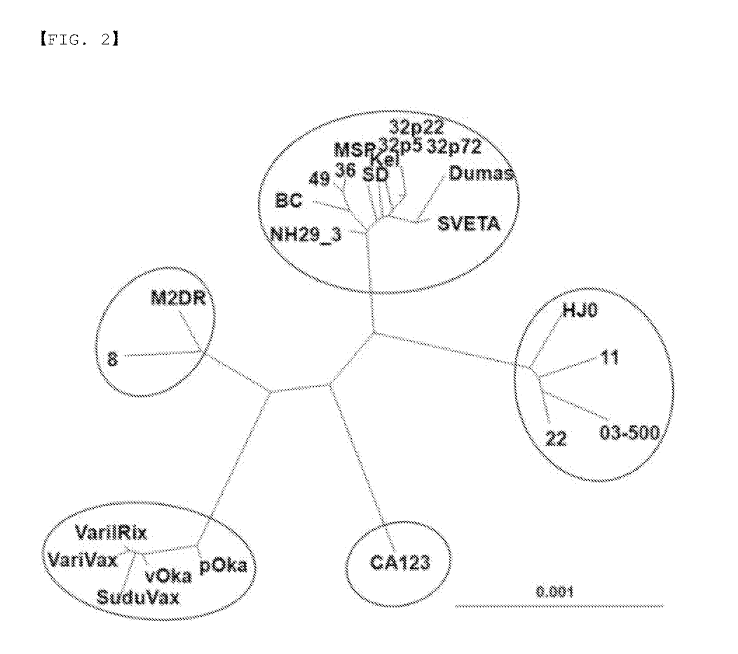

The phylogenetic tree was constructed by the maximum-likelihood (ML) method based on the entire nucleotide sequences. As a result, virus strains (vOka, Varilrix, and Varivax) including the MAV/06 virus strain, which were virus strains of 4 live attenuated virus vaccines, formed a cluster, and M2DR virus strain and 8 virus strains formed a cluster adjacent thereto, as shown in FIG. 2. In addition, CA123 virus strain independently formed a third cluster, 11, 22, 03-500, and HJ0 virus strains formed a fourth cluster, and the rest of the clinical virus strains formed the other cluster.

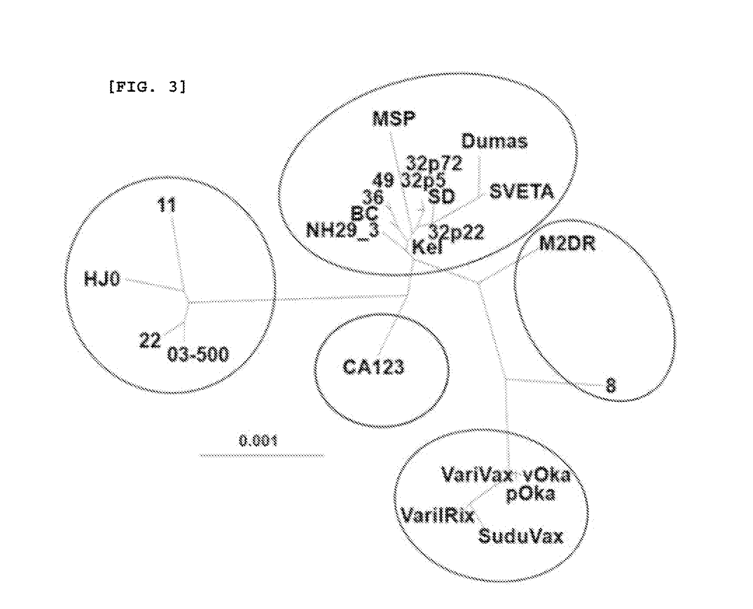

As the result obtained by constructing the phylogenetic tree using the maximum-likelihood (ML) method based on the non-coding nucleotide sequences, the MAV/06 virus strain isolated from a Korean patient and Oka virus strain isolated from a Japanese patient formed a unique cluster (Japan-Korean (J-K) cluster) as shown in FIG. 3. The pOka virus strain was positioned between 4 vaccine virus strains (vOka, Varilrix, Varivax, and MAV/06) and 19 clinical virus strains in the phylogenetic tree of the 24 VZV virus strains constructed based on the entire nucleotide sequences and common amino acid sequences but positioned between the vaccine virus strains in the phylogenetic tree of the 24 VZV virus strains constructed based on the non-coding nucleotide sequences. That is, in other words, it was confirmed that the 4 vaccine virus strains (vOka, Varilrix, Varivax, and MAV/06) formed a subclade in the Japan-Korean (J-K) cluster in the phylogenetic tree of the 24 VZV virus strains constructed based on the entire nucleotide sequences and common amino acid sequences but did not form the subclade in the phylogenetic tree of the 24 VZV virus strains constructed based on the non-coding nucleotide sequences.

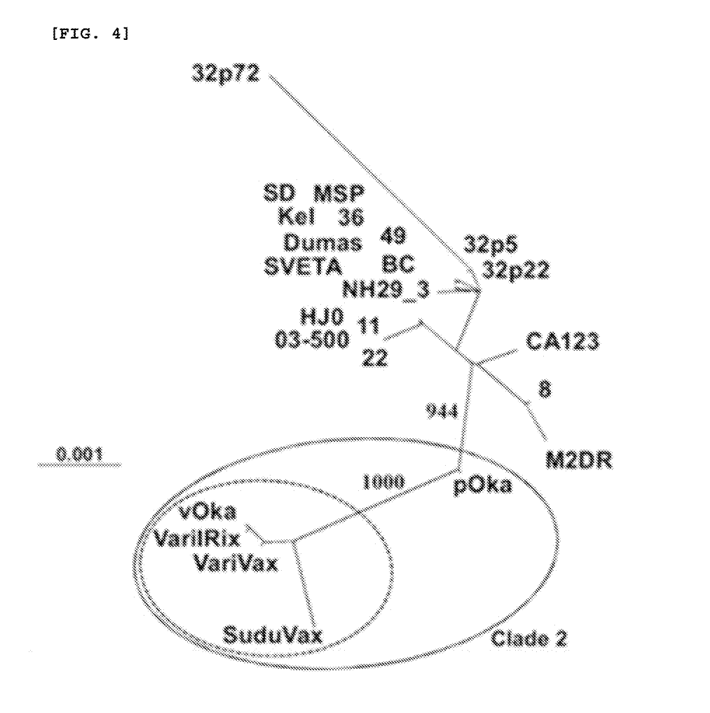

In order to confirm main ORFs for distinguishing the vaccine virus strains and clinical virus strains from each other, 74 phylogenetic trees according to the ORFs were constructed. Among them, vaccine virus strain-specific phylogenetic trees were confirmed in 12 ORFs, as shown in FIG. 4. As the ORFs capable of obtaining the vaccine virus strain-specific phylogenetic tree, there are ORFs 0, 1, 6, 18, 31, 35, 39, 59, 62, 64, 69, and 71.

(3) Reiteration Sequences and Polymorphism Analysis of Length of Genome

Lengths of the ORFs of the VZV virus strains were hardly changed. It may be confirmed that among 74 ORFs of the VZV virus strains, there was almost no difference in a length of the ORF 63 between the VZV virus strains. As shown in the following Table 2, it may be confirmed that polymorphism of the length of 3 ORFs (ORFs 11, 14, and 22) between the strains was relatively high due to lengths of the reiteration sequences.

TABLE 2
REITERATION SEQUENCE OF VZV VIRUS STRAINS

| | Length (bp) | | | | | | | | | Rank⁴ | Rank⁴ |
|---|---|---|---|---|---|---|---|---|---|---|---|
| Strain | R1 | R2 | R3 | R4a | R4b | R5 | R¹ | G² | G − R³ | (R) | (G) |
| Dumas | 303 | 326 | 72 | 146 | 146 | 200 | 1,193 | 124,884 | 123,691 | 9 | 10 |
| M2DR | 303 | 284 | 27 | 146 | 146 | 200 | 1,106 | 124,770 | 123,664 | 3 | 2 |
| CA123 | 261 | 284 | 36 | 146 | 146 | 200 | 1,073 | 124,771 | 123,698 | 1 | 3 |
| SD | 258 | 326 | 72 | 281 | 281 | 200 | 1,418 | 125,087 | 123,669 | 16 | 17 |
| Kel | 321 | 326 | 72 | 389 | 389 | 200 | 1,697 | 125,374 | 123,677 | 21 | 22 |
| 11 | 225 | 326 | 225 | 362 | 362 | 200 | 1,700 | 125,370 | 123,670 | 22 | 21 |
| 22 | 195 | 284 | 72 | 200 | 200 | 200 | 1,151 | 124,868 | 123,717 | 8 | 8 |
| 03-500 | 225 | 284 | 657 | 92 | 92 | 200 | 1,550 | 125,239 | 123,689 | 20 | 20 |
| 36 | 321 | 326 | 72 | 227 | 227 | 200 | 1,373 | 125,030 | 123,657 | 13 | 13 |
| 49 | 321 | 410 | 54 | 200 | 200 | 200 | 1,385 | 125,041 | 123,656 | 14 | 14 |
| 8 | 258 | 620 | 36 | 335 | 335 | 200 | 1,784 | 125,451 | 123,667 | 23 | 23 |
| 32p5 | 243 | 326 | 54 | 227 | 227 | 200 | 1,277 | 124,945 | 123,668 | 12 | 12 |
| 32p22 | 291 | 326 | 36 | 281 | 281 | 200 | 1,415 | 125,084 | 123,669 | 15 | 16 |
| 32p72 | 291 | 326 | 117 | 281 | 281 | 200 | 1,496 | 125,169 | 123,673 | 19 | 19 |
| NH29_3 | 258 | 326 | 51 | 146 | 146 | 200 | 1,127 | 124,811 | 123,684 | 4 | 4 |
| SVETA | 243 | 326 | 72 | 146 | 146 | 200 | 1,133 | 124,813 | 123,680 | 5 | 5 |
| MSP | 258 | 368 | 90 | 146 | 146 | 200 | 1,208 | 124,883 | 123,675 | 10 | 9 |
| BC | 258 | 326 | 612 | 200 | 200 | 200 | 1,796 | 125,459 | 123,663 | 24 | 24 |
| HJ0 | 225 | 326 | 72 | 146 | 254 | 200 | 1,223 | 124,928 | 123,705 | 11 | 11 |
| pOka | 225 | 326 | 36 | 281 | 281 | 312 | 1,461 | 125,125 | 123,664 | 18 | 18 |
| vOka | 258 | 326 | 27 | 254 | 254 | 312 | 1,431 | 125,078 | 123,647 | 17 | 15 |
| VarilRix | 258 | 326 | 72 | 146 | 146 | 200 | 1,148 | 124,821 | 123,673 | 6 | 7 |
| VariVax | 258 | 326 | 72 | 146 | 146 | 200 | 1,148 | 124,815 | 123,667 | 6 | 6 |
| MAV06 | 273 | 326 | 99 | 92 | 92 | 200 | 1,082 | 124,759 | 123,677 | 2 | 1 |
| Avg | 263.8 | 336.5 | 116.9 | 209.0 | 213.5 | 209.3 | 1,349 | 125,024 | 23,675 | | |
| Stdev | 33.8 | 65.8 | 164.5 | 83.0 | 82.4 | 31.6 | 229.7 | 224.1 | 16.0 | | |
| CV | 0.128 | 0.196 | 1.407 | 0.397 | 0.386 | 0.151 | 0.170 | 0.00179 | 0.00013 | | |

¹Sum of R sequences
²Length of the genome
³Length of the genome after deletion of R sequences
⁴Rank in the order of length from the shortest to the longest

That is, R1 was positioned in the ORF 11 and formed by combining various sequences of an 18-bp element (consensus: GGACGCGATCGACGACGA (SEQ ID NO: 7)) and a 15-bp element (consensus: GGGAGAGGCGGAGGA (SEQ ID NO: 8)). Since a 15-bp element in the ORF 11 was additionally repeated in the MAV/06 virus strain, the length thereof was different from that of the Oka vaccine virus strain.

In addition, R2 was positioned in the ORF 14 and configured various 42-bp elements (consensus: ACCTCGGCCGCTT/aCCCGAAAG/taCCCGATCCCGCCGTCGCGCCC (SEQ ID NO: 9), here, a small letter indicates a small deviation of the sum), and a part thereof (32-bp element) was further included in the R2. In the MAV/06 virus strain, the 42-bp element in the ORF 11 was repeated 7 times as in the Oka vaccine virus strain.

Further, R3 was positioned at the center of S-end of the ORF 22, and a 9-bp element was repeatedly replicated. A consensus sequence of the 9-bp element was GC/tCCGC/tG/cCA/g (here, a small letter indicates a small deviation of the sum). A repetition number (n) was significantly different between the strains. In the 03-500 virus strain, the repetition number was 73, and in the vOka virus strain, the repetition number was 3. In the MAV/06 virus strain, a repetition number of a 9-bp element was 11, but in the Varilrix and Varivax virus strains, the repetition number was 8.

In addition, R4 and R5 were non-coding regions. R4 was configured of a 27-bp element (consensus: CCCCGCCGATGGGGAGGGGGCGCGGTA (SEQ ID NO: 10)), and a part (11-bp element) thereof was further included in the R4. R4 (R4a) was positioned between the ORFs 62 and 63 in the IRS, and R4b complementary thereto was positioned between the ORFs 70 and 71 in the TRS. A length of the R4a was the same as that of the R4b, but in the case of the HJO virus strain, the lengths of the R4a and R4b were different. Since in the R4b of the HJO virus strain, the 27-bp element was further repeated 4 times, the length of the R4b was longer than that of the R4a by 108 bp. In the case of the Varilrix and Varivax virus strains, the 27-bp element was further repeated 5 times. In the MAV/06 virus strain, a 27-bp element was further repeated 3 times.

R5 was positioned between the ORFs 60 and 61 and configured of an 88-bp element including a 24-bp element. The two elements were different from other reiteration sequences. Two types of reiteration sequences were present. The MAV/06 virus strain included two 88-bp elements and one 24-bp element as most of the virus strains, but the vOka and pOka virus strains included three 88-bp elements and two 24-bp elements.

It was confirmed by comparing the lengths of the genome sequences of the 24 VZV virus strains with the lengths of the reiteration sequences that the polymorphism of the lengths of the genomes of the VZV virus strains was due to the length of the reiteration sequence. The BC virus strain includes the longest reiteration sequence as the longest genome, but the MAV/06 virus strain includes the second-shortest reiteration sequence as the shortest genome. A length (700 bp) between the longest genome and the shortest genome was similar to a difference (724 bp) between the longest reiteration sequence and the shortest reiteration sequence. If the reiteration sequence was deleted from the genome, the length of the genome was decreased together with a change in coefficient of variation (CV). When the reiteration sequence was deleted from the entire genome, 0.000129 was deleted, and the CV was calculated as 0.00179.

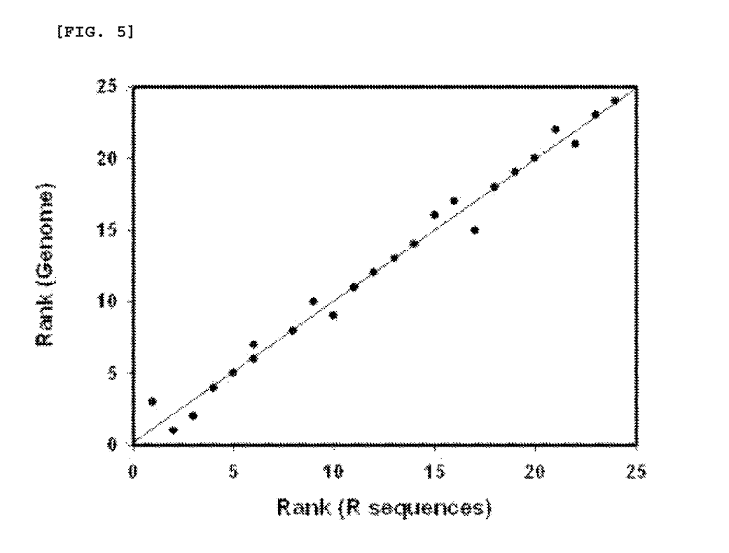

Actually, as shown in FIG. 5, when an order of the length of the genome was set as an order of the length of the reiteration sequences, correlation coefficient (r²) was 0.9854, and a linear relationship between the lengths of the genomes of the VZV virus strains and the reiteration sequences was almost established.

(4) Origin of Replication

An origin of replication (OR1) sequence of the VZV virus strains was positioned between the ORFs 62 and 63. A length of the OR1 sequence in the 36 virus strain was 80 bp and a length of the OR1 sequence in the HJO virus strain was 108 bp since repetition numbers of TA and GA dinucleotides in arrangement of the OR1 sequence were different from each other.

As shown in the following Table 3, the OR1 sequence in the MAV/06 virus strain was positioned between the 110,080th and 110,183th positions of the genome, and 15 GA tandem repeats and 11 TA tandem repeats were included therein.

TABLE 3

| Strain | Start | End | Length | (TA)n¹ | (GA)m² |
|---|---|---|---|---|---|
| Dumas | 110,166 | 110,263 | 98 | 15 | 8 |
| M2DR | 110,052 | 110,149 | 98 | 15 | 8 |
| CA123 | 110,050 | 110,147 | 98 | 15 | 8 |
| SD | 110,250 | 110,337 | 88 | 11 | 7 |
| Kel | 110,431 | 110,514 | 84 | 9 | 7 |
| 11 | 110,455 | 110,538 | 84 | 8 | 8 |
| 22 | 110,075 | 110,180 | 106 | 13 | 14 |
| 03-500 | 110,580 | 110,667 | 88 | 11 | 7 |
| 36 | 110,262 | 110,341 | 80 | 7 | 7 |
| 49 | 110,300 | 110,379 | 80 | 7 | 7 |
| 8 | 110,541 | 110,632 | 92 | 13 | 7 |
| 32p5 | 110,171 | 110,254 | 84 | 9 | 7 |
| 32p22 | 110,256 | 110,339 | 84 | 9 | 7 |
| 32p72 | 110,335 | 110,420 | 86 | 10 | 7 |
| NH29_3 | 110,094 | 110,191 | 98 | 15 | 8 |
| SVETA | 110,101 | 110,194 | 94 | 13 | 8 |
| MSP | 110,176 | 110,265 | 90 | 12 | 7 |
| BC | 110,708 | 110,795 | 88 | 11 | 7 |
| HJO | 110,085 | 110,192 | 108 | 11 | 17 |
| pOka | 110,268 | 110,371 | 104 | 11 | 15 |
| vOka | 110,264 | 110,359 | 96 | 8 | 14 |
| VarilRix | 110,096 | 110,191 | 96 | 8 | 14 |
| VariVax | 110,093 | 110,190 | 98 | 8 | 15 |
| MAVO6 | 110,080 | 110,183 | 104 | 11 | 15 |

¹THE NUMBER OF TA TANDEM REPEATS
²THE NUMBER OF GA TANDEM REPEATS

The numbers of tandem repeats in 110,235 positions of the genome were different, but in the Oka vaccine virus strain and MAV/06 virus strain, generally, A was substituted with G in the GA tandem repeats at one position of the nucleotide positions corresponding to 110,235 positions in the Dumas virus strain, which is a reference virus strain. The OR1 sequence was accurately complementarily duplicated between the ORFs 70 and 71 except for in the HJO and Varilrix virus strains: (TA)₃(GA)₂ was deleted in the HJO virus strain, and one GA tandem repeat was deleted in the Varilrix virus strain.

(5) Analysis of Characteristics of ORF of MAV/06 Virus Strain

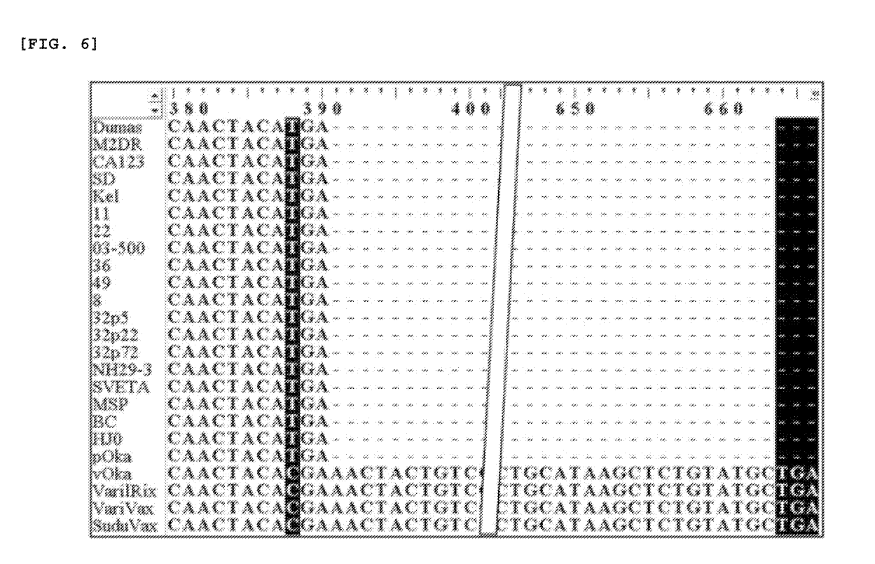

A length of the ORFO of the MAV/06 virus strain was increased due to mutation. TGA (388-390th nucleotide position), which is a termination codon, was mutated into CGA coding arginine (Agr). TGA estimated as the termination codon was founded at a lower end region overlapped with the ORF1. The expanded ORFO encoded a new protein having 221 amino acid residues. The same mutation was confirmed in other similar vOka, Varilrix, and Varivax vaccine virus strains (FIG. 6).

Among all of the clinical virus strains, the pOka virus strain included an ORFO coding 129 amino acids and having a length of 390 bp.

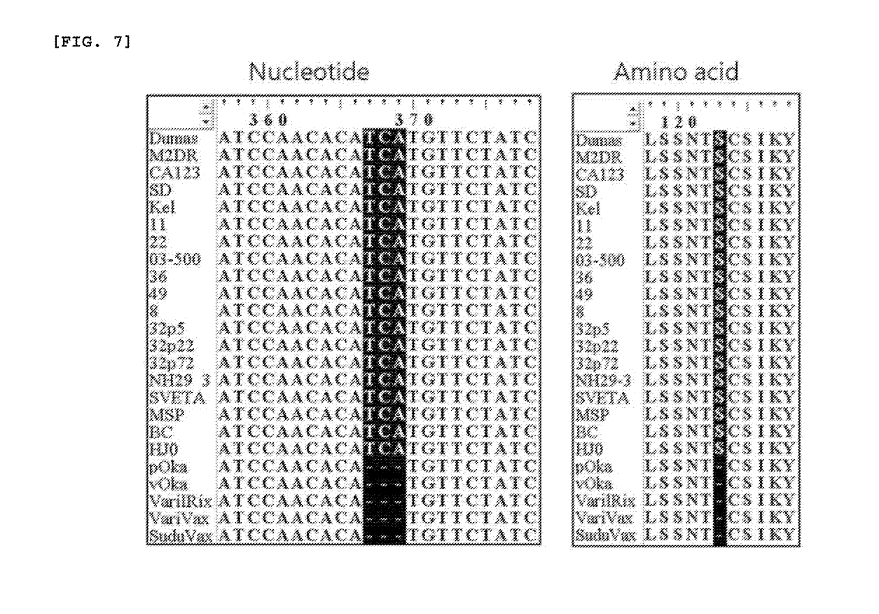

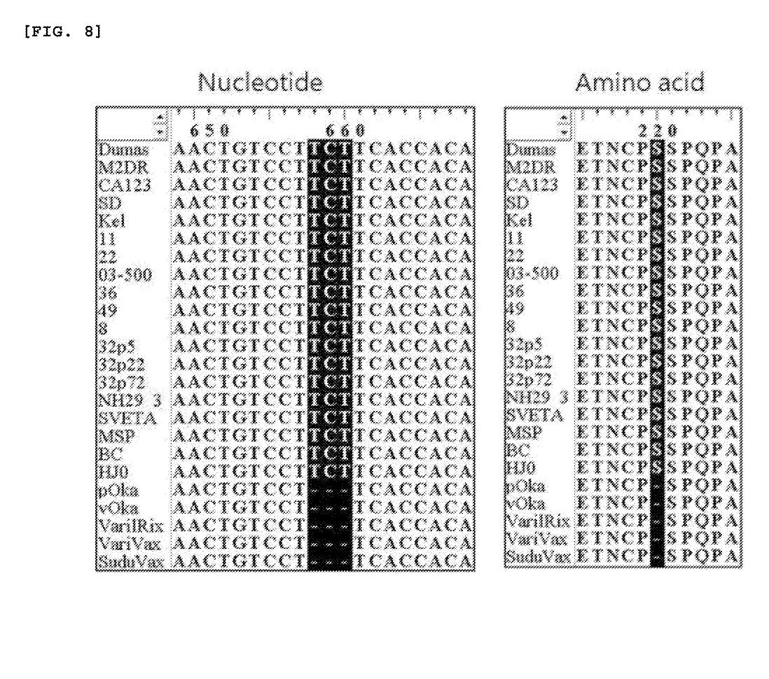

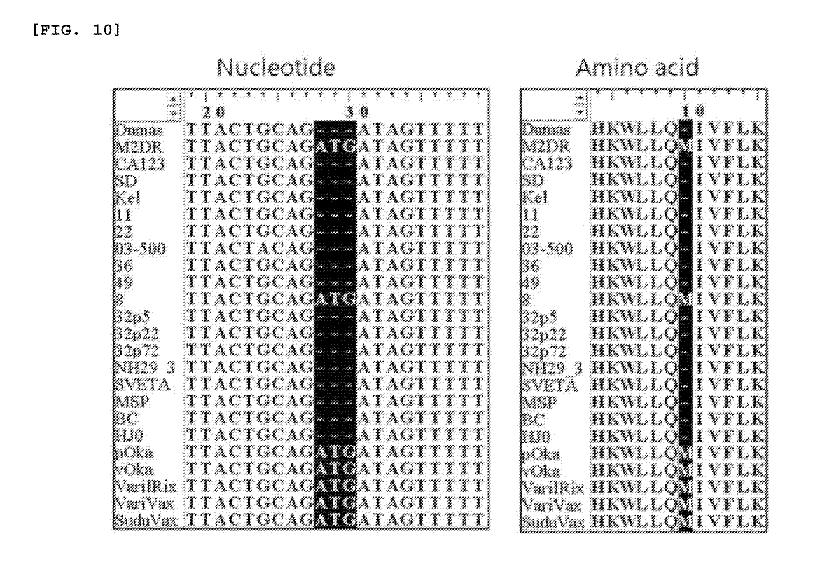

As compared with the Dumas virus strain that is the reference virus strain, since TCT at the 658th to 660th position and TCA at the 367th to 369th position were deleted from the ORF17 (SEQ ID NO. 3) and the ORF56 (SEQ ID NO. 5) of the MAV/06 virus strain, respectively, the lengths of the ORF17 and the ORF56 were shortened by 3 bp (FIGS. 7 and 8). All of the above-mentioned deletions caused deletion of a serine (S) amino acid residue. On the other hand, in the ORF60 (SEQ ID NO. 6) of the MAV/06 virus strain, three nucleotides (ATG) were inserted at a 28th position (FIG. 10). Interestingly, one insertion and two deletions were found in all of the Oka vaccine virus strains including the pOka virus strain.

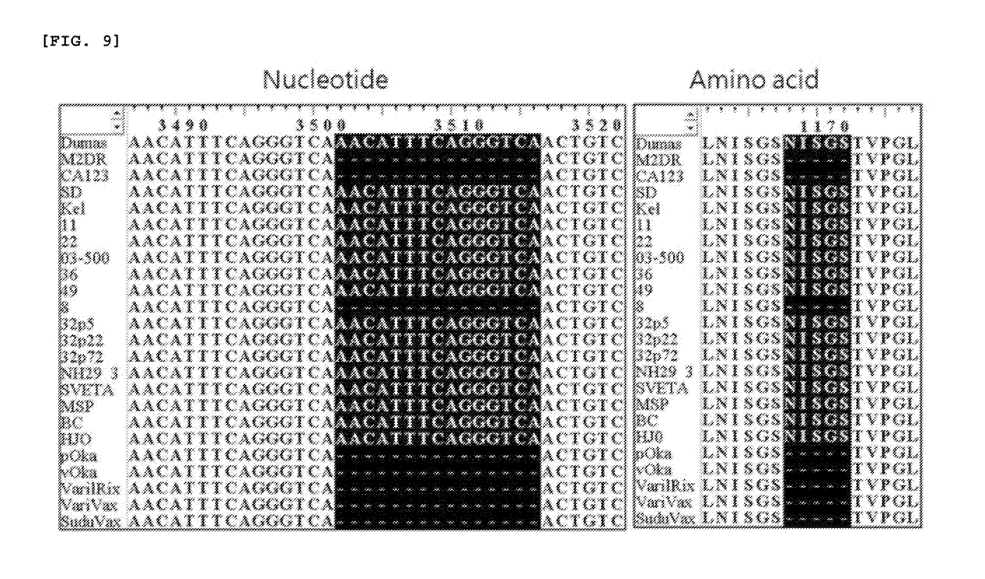

In addition, in the MAV/06 virus strain as well as the Oka vaccine virus strains, the length of the ORF29 (SEQ ID NO. 4) was shorter than those of the ORF29 in the Dumas strain and most of the clinical virus strains by 15 bp (AACATTTCAGGGTCA (SEQ ID NO: 11)). The Dumas strain, which was the reference virus strain, and most of the clinical virus strains continuously and repetitively included a 15 bp-sequence. In the M2DR, CA123, and 8 clinical virus strains, the length of the ORF60 was the same as that of the ORF60 in the Oka vaccine virus strain and the MAV/06 virus strain (FIG. 9).

[Sequence List Pretext]

SEQ ID NO. 1 is a sequence of genome DNA Of VZV MAV/06 strain.

SEQ ID NO. 2 is an ORFO of the genome DNA Of VZV MAV/06 strain.

SEQ ID NO. 3 is an ORF17 of the genome DNA Of VZV MAV/06 strain.

SEQ ID NO. 4 is an ORF29 of the genome DNA Of VZV MAV/06 strain.

SEQ ID NO. 5 is an ORF56 of the genome DNA Of VZV MAV/06 strain.

SEQ ID NO. 6 is an ORF60 of the genome DNA Of VZV MAV/06 strain.

1. A genome DNA of Varicella-zoster virus (VZV) MAV/06 strain having a base sequence of SEQ ID NO. 1.

2. An open reading frame (ORF) of genome DNA of VZV MAV/06 strain selected from an ORFO having a base sequence of SEQ ID NO. 2, an ORF17 having a base sequence of SEQ ID NO. 3, an ORF29 having a base sequence of SEQ ID NO. 4, an ORF56 having a base sequence of SEQ ID NO. 5, and an ORF60 having a base sequence of SEQ ID NO.

3. A protein coded by the open reading frame of genome DNA of VZV MAV/06 strain of claim 1.

4. A recombinant expression vector comprising: genome DNA of VZV MAV/06 strain having a base sequence of SEQ ID NO. 1 or an ORF selected from an ORFO having a base sequence of SEQ ID NO. 2, an ORF17 having a base sequence of SEQ ID NO. 3, an ORF29 having a base sequence of SEQ ID NO. 4, an ORF56 having a base sequence of SEQ ID NO. 5, and an ORF60 having a base sequence of SEQ ID NO. 6.

5. Host cells transformed by the recombinant expression vector of claim 4.

6. The host cells of claim 5, wherein they are selected from animal cells, plant cells, yeasts, Escherichia coli, and insect cells.

7. The host cells of claim 6, wherein they are selected from monkey kidney cells 7 (COS7), NSO cells, SP2/O cells, Chinese hamster ovary (CHO) cells, WI38 cells, baby hamster kidney (BHK) cells, Madin-Darby canine kidney (MDCK) cells, myeloma cell lines, HuT 78 cells, HEK293 cells, Escherichia coli, Bacillus subtilis, Streptomyces sp., Pseudomonas sp., Proteus mirabilis or Staphylococcus sp., Aspergillus sp., Pichia pastoris, Saccharomyces cerevisiae, Schizosaccharomyces sp., and Neurospora crassa.

8. A method of preparing a protein of VZV MAV/06 strain coded by any one base sequence selected from SEQ ID NOS. 1 to 6.

9. A vaccine composition comprising: the protein of VZV MAV/06 strain of claim 8 as an active ingredient.

10. The vaccine composition of claim 9, further comprising a pharmaceutically acceptable adjuvant serving to promote formation of an antibody at the time of injecting the adjuvant into the body.

11. The vaccine composition of claim 10, wherein the adjuvant is at least one selected from an aluminum salt (Al(OH)3 or ALPO4), squalene, sorbitane, polysorbate 80, CpG, liposome, cholesterol, monophosphoryl lipid (MPL) A, and glucopyranosyl lipid A (GLA).

12. A protein coded by the open reading frame of genome DNA of VZV MAV/06 strain of claim 2.
